(19) United States
(12) Patent Application Publication
(10) Pub. No.: US 2026/0297505 A1
(43) Pub. Date: Oct. 1, 2026

(54) PRODUCTION OF CARBON COMPOUNDS

(71) Applicant: C3 BIOTECHNOLOGIES LIMITED, Lancaster (GB)

(72) Inventor: Robin HOEVEN, Lancaster (GB)

(21) Appl. No.: 19/479,676

(22) PCT Filed: May 1, 2024
(86) PCT No.: PCT/EP2024/061994

§ 371 Date: Oct. 28, 2025

(30) Foreign Application Priority Data
May 2, 2023 (GB) 2306449.6

(51) Int. Cl. C12N 1/205; C02F 3/34; C02F 101/10; C12N 9/88; C12N 15/52; C12P 5/02; C12R 1/01

(52) U.S. Cl. CPC C12N 1/205; C02F 3/342; C12N 9/88; C12N 15/52; C12P 5/02; C12Y 401/01; C12Y 402/03; C02F 2101/101; C12N 2800/101; C12R 2001/01

(57) ABSTRACT

Disclosed herein are methods for the production of carbon compounds, Halomonas rowanensis deposited with European Collection of Authenticated Cell Cultures on 10 Feb. 2023 under accession number 23021001, and methods using Halomonas rowanensis.

This application claims priority from GB2306449.6 filed 2 May 2023, the contents and elements of which are herein incorporated by reference for all purposes.

### FIELD OF THE INVENTION

The present invention relates to the fields of molecular biology and biotechnology and particularly, although not exclusively, to microorganisms for the production of carbon compounds.

### BACKGROUND

The implementation of a sustainable bio-based economy relies on the development of efficient and high titre microbial bioprocesses, with market price parity to low priced fossil fuel-derived alternatives.

Traditional fermentations often require expensive raw materials, large quantities of fresh water, high energy usage and challenging downstream processes (Chen and Jiang, 2018. Curr. Opin. Biotechnol. 50, 94-100). Therefore, a step change is needed in traditional microbial bioprocess development to overcome the cost competitiveness hurdle towards commercially relevant bioproduction.

A Halomonas bluephagenesis strain TQ10 has been modified for the production of bio-LPG (propane, butane and isobutane) on a laboratory scale (Amer et al., 2020a. Biotechnol. Biofuels 13, 125; Amer et al., 2020b. Energy Environ. Sci. 13, 1818-1831). An early stage technoeconomic analysis of the process was based on using locally sourced seawater and glycerol as an additional carbon source (Amer et al., 2020b. Energy Environ. Sci. 13, 1818-1831). In addition, it has been demonstrated that a lower carbon approach to propane production is possible by engineering the photosynthetic cyanobacterial microorganism Synechocystis sp. PCC6803 to generate propane directly from CO₂. However, the photosynthetic production of propane requires light which may be a barrier to cost-effective propane production through the photosynthetic route.

Chemoautotrophic growth of one strain of Halomonas has been demonstrated (Mishra et al., 2017. Process Biochem. 55, 133-145). However, this newly identified strain was not genetically modified for carbon compound production.

The present invention has been devised in light of the above considerations.

### SUMMARY OF THE INVENTION

In one aspect, the present invention provides a Halomonas rowanensis bacterium, wherein the bacterium is the strain as deposited with European Collection of Authenticated Cell Cultures on 10 Feb. 2023 under accession number 23021001 or a derivative of the deposited Halomonas rowanensis.

Halomonas rowanensis accession number ECCD23021001.C is wild-type native Halomonas rowanensis which was isolated from a brine spring in Cheshire, UK.

A derivative may alternatively be described as a mutant or a variant. Therefore, in some embodiments, the organism is a mutant of the Halomonas rowanensis deposited with European Collection of Authenticated Cell Cultures on 10 Feb. 2023 under accession number 23021001. In some embodiments, the organism is a variant of the Halomonas rowanensis deposited with European Collection of Authenticated Cell Cultures on 10 Feb. 2023 under accession number 23021001.

In some embodiments, the derivative expresses one or more heterologous genes. In some embodiments, the derivative expresses one or more heterologous genes for the production of a carbon compound.

In some embodiments, the derivative expresses a heterologous fatty acid photodecarboxylase. In some embodiments, the fatty acid photodecarboxylase comprises an amino acid sequence having at least 70% sequence identity to SEQ ID NO:1. In some embodiments, the fatty acid photodecarboxylase comprises, or consists, of an amino acid sequence having at least 75%, 80%, 85%, 90%, 95%, 99%, or 100% sequence identity to SEQ ID NO:1.

In some embodiments, the derivative expresses a fatty acid photodecarboxylase comprising an amino acid substitution at a position corresponding to G462 of SEQ ID NO:1. In some embodiments, the amino acid substitution is G462I or G462V. In some embodiments, the derivative expresses a pHALP7-CvFAP_{G462V} or pHALP102-CvFAP_{G462V} plasmid. In some embodiments, the derivative expresses a pHALP7-CvFAP_{G462I} or pHALP102-CvFAP_{G462I} plasmid.

An exemplary derivative of the deposited Halomonas rowanensis (accession number 23021001) is a genetically modified organism which contains a pHALP7_CvFAP_{G462V} plasmid which confers fatty acid photodecarboxylase activity for propane production. The pHALP7_CvFAP_{G462V} plasmid contains a gene encoding fatty acid photodecarboxylase (CvFAP) from Chlorella variabilis NC64A variant G462V (Amer et al., 2020b). The plasmid also contains a broad-host-range origin of replication (oriV) from Pseudomonas aeruginosa, as well as an origin of transfer (oriT) and a P7 promoter (Trisrivirat et al., 2020, https://doi.org/10.1093/synbio/ysaa022). The plasmid map for pHALP7_CvFAP_{G462V} is shown in FIG. 1.

In another aspect, the present invention provides a microbial culture comprising Halomonas rowanensis deposited with European Collection of Authenticated Cell Cultures on 10 Feb. 2023 under accession number 23021001 or a derivative of the deposited Halomonas rowanensis.

In another aspect, the present invention provides a method of culturing Halomonas rowanensis deposited with European Collection of Authenticated Cell Cultures on 10 Feb. 2023 under accession number 23021001, or a derivative of the deposited Halomonas rowanensis.

In some embodiments, the Halomonas rowanensis or the derivative of the deposited Halomonas rowanensis is cultured in the presence of carbon dioxide. In some embodiments, the method further comprises isolation of a carbon compound from the culture.

In another aspect, the present invention provides a method of producing a carbon compound, the method comprising culturing a Halomonas cell in the presence of carbon dioxide, wherein the Halomonas cell is capable of fixing carbon dioxide, further comprising isolation of a carbon compound from the culture.

In some embodiments, the fixed carbon dioxide is metabolically converted to another carbon compound. In some embodiments the Halomonas cell is a chemoautotroph. In some embodiments the Halomonas cell is a facultative chemoautotroph. In some embodiments, the Halomonas cell is cultured in the presence of sulfur containing compounds. In some embodiments, the Halomonas cell is cultured in the presence of a reduced sulfur compound. In some embodiments, the Halomonas cell is cultured in the presence of thiosulfate, sulfuric acid, sulfur, sulfate, and/or sulfite.

In some embodiments, the carbon compound is a C2-C17 carbon compound, biofuel, bio-LPG, and/or a bio-alkane. In some embodiments, the carbon compound is propane, butane, isobutane, ethanol, linalool, or ectoine.

In some embodiments, the Halomonas expresses one or more heterologous genes. In some embodiments, the Halomonas expresses 1, 2, 3, 4, 5, 6, 7, 8, 9, 10, 11, 12, 13, 14, 15, 16, 17, 18, 19, 20, 25, 30, 35 or 40 heterologous genes.

In some embodiments, the Halomonas expresses one or more heterologous genes for the production of a carbon compound. In some embodiments, the Halomonas expresses 1, 2, 3, 4, 5, 6, 7, 8, 9, 10, 11, 12, 13, 14, 15, 16, 17, 18, 19, 20, 25, 30, 35 or 40 heterologous genes for the production of a carbon compound.

In some embodiments, the Halomonas expresses one or more heterologous genes for C2-C17 carbon compound production. In some embodiments, the Halomonas expresses a heterologous pathway for C2-C17 carbon compound production. In some embodiments the Halomonas expresses one or more heterologous genes for propane and/or linalool production. In some embodiments the Halomonas expresses a heterologous pathway for propane and/or linalool production.

In some embodiments, the Halomonas is modified with one or more heterologous gene for C2-C17 carbon compound production. In some embodiments, the Halomonas is modified with heterologous pathway for C2-C17 carbon compound production. In some embodiments the Halomonas is modified with a heterologous pathway for propane, linalool, and/or amine production.

In some embodiments, the Halomonas cell expresses a heterologous fatty acid photodecarboxylase. In some embodiments, the Halomonas cell expresses a fatty acid photodecarboxylase from Chlorella variabilis NC64A (known as CvFAP or CvPAS). In some embodiments, the Halomonas is modified to express a heterologous fatty acid photodecarboxylase. In some embodiments, the Halomonas is modified to express photoalkane synthase from Chlorella variabilis NC64A (known as CvFAP or CvPAS). In some embodiments, the fatty acid photodecarboxylase comprises, or consists, of an amino acid sequence having at least 70%, 75%, 80%, 85%, 90%, 95%, 99%, or 100% sequence identity to SEQ ID NO:1.

In some embodiments, the Halomonas has been transformed with a pHALP7_CvFAP_{G462V}, a pHALP7_CvFAP_{G462V}, a pHALP7_CvFAP_{G462I} and/or a pHALP102_CvFAP_{G462I} plasmid.

In some embodiments, the Halomonas cell expresses a heterologous aldehyde dehydrogenase. In some embodiments, the Halomonas is modified to express a heterologous aldehyde dehydrogenase. In some embodiments, the Halomonas is modified to express Clostridium acetobutylicum ATCC 824 enzyme AdhE2 and/or Clostridium beijerinckii BALDH. In some embodiments, the aldehyde dehydrogenase comprises, or consists, of an amino acid sequence having at least 70%, 75%, 80%, 85%, 90%, 95%, 99%, or 100% sequence identity to SEQ ID NO: 16.

In some embodiments, the Halomonas cell expresses a heterologous aldehyde deformylating oxygenase (ADO). In some embodiments, the Halomonas is modified to express a heterologous aldehyde deformylating oxygenase. In some embodiments, the Halomonas is modified to express aldehyde deformylating oxygenase from Procholorococcus marinus st. MIT9313 (ADO). In some embodiments, the aldehyde deformylating oxygenase comprises, or consists, of an amino acid sequence having at least 70%, 75%, 80%, 85%, 90%, 95%, 99%, or 100% sequence identity to SEQ ID NO: 23.

In some embodiments, the Halomonas cell expresses a heterologous ferredoxin. In some embodiments, the Halomonas is modified to express a heterologous ferredoxin. In some embodiments, the Halomonas is modified to express ferredoxin from Synechocystis sp. PCC6803 (PetF). In some embodiments, the ferredoxin comprises, or consists, of an amino acid sequence having at least 70%, 75%, 80%, 85%, 90%, 95%, 99%, or 100% sequence identity to SEQ ID NO:38.

In some embodiments, the Halomonas cell expresses a heterologous linalool synthase. In some embodiments, the Halomonas is modified to express a heterologous linalool synthase. In some embodiments, the Halomonas is modified to express a heterologous linalool synthase from Streptomyces clavuligerus. In some embodiments, the linalool synthase comprises, or consists, of an amino acid sequence having at least 70%, 75%, 80%, 85%, 90%, 95%, 99%, or 100% sequence identity to SEQ ID NO: 30, SEQ ID NO: 31, SEQ ID NO: 32, and/or SEQ ID NO: 33.

In some embodiments, the linalool synthase is a linalool synthase variant comprising an amino acid substitution at a position corresponding to L72 and/or V214 of SEQ ID NO:30. In some embodiments, the amino acid substitution is L72M, V214L, or V214I. In some embodiments, the linalool synthase variant comprises L72M and V214I substitutions. In some embodiments, the linalool synthase variant comprises L72M and V214L substitutions. In some embodiments, the linalool synthase variant comprises, or consists, of an amino acid sequence having at least 70%, 75%, 80%, 85%, 90%, 95%, 99%, or 100% sequence identity to SEQ ID NO: 30, SEQ ID NO: 31, SEQ ID NO: 32, and/or SEQ ID NO: 33. In some embodiments, the Halomonas may express (either naturally or by genetic modification) one or more enzymes which can convert acetyl-CoA into isopentenyl pyrophosphate (IPP), for example by way of the mevalonate-dependent (MVA) pathway. In embodiments, the one or more enzymes may include, for example, acetoacetyl-CoA thiolase (AtoB, EC 2.3.1.9) (from Escherichia coli for example), hydroxymethylglutaryl-CoA synthase (HMGS, EC 2.3.3.10) (from Saccharomyces cerevisiae for example), hydroxymethylglutaryl-CoA reductase (HMGR, EC 1.1.1.34) (from Saccharomyces cerevisiae for example), mevalonate kinase (MK, EC 2.7.1.36) (from Saccharomyces cerevisiae for example), phosphomevalonate kinase (PMK, EC 2.7.4.2) (from Saccharomyces cerevisiae for example), phosphomevalonate decarboxylase (PMD, EC 4.1.1.33) (from Saccharomyces cerevisiae for example) and/or isopentenyl diphosphate isomerase (idi, EC 5.3.3.2) (from Escherichia coli for example).

In some embodiments, the Halomonas may express (either naturally or by genetic modification) one or more enzymes which can convert pyruvate into dimethylallyl pyrophosphate (DMAPP), for example by way of the methylerythritol 4-phosphate (MEP) pathway. In some embodiments, the one or more enzymes may include, for example, 1-deoxyxylulose-5-phosphate synthase (DXS, EC 2.2.1.7), 1-deoxyxylulose-5-phosphate reductoisomerase (DXR, IspC, EC 1.1.1.267), 2-C-methyl-D-erythritol 4-phosphate cytidylyltransferase (YgbP, IspD, EC 2.7.7.60), 4-(cytidine 5′-diphospho)-2-C-methyl-D-erythritol kinase (YchB, IspE, EC 2.7.1.148), (E)-4-hydroxy-3-methylbut-2-enyl-diphosphate synthase (GcpE, IspG, EC 1.17.7.1) and/or 4-hydroxy-3-methylbut-2-en-1-yl diphosphate reductase (LytB, IspH, EC 1.17.7.4). In embodiments, such enzymes may be from E. coli, for example.

In some embodiments, the Halomonas may express (either naturally or by genetic modification) one or more enzymes which can convert isopentenyl pyrophosphate (IPP) and dimethylallyl diphosphate (DMAPP) into geranyl pyrophosphate (GPP). In some embodiments, the Halomonas cell expresses a heterologous geranyl diphosphate synthase (GPPS). In some embodiments, the Halomonas cell expresses a heterologous geranyl diphosphate synthase (GPPS) from Abies grandis. In some embodiments, the GPPS comprises, or consists, of an amino acid sequence having at least 70%, 75%, 80%, 85%, 90%, 95%, 99%, or 100% sequence identity to SEQ ID NO: 39 and/or SEQ ID NO: 40.

In some embodiments, the derivative of Halomonas rowanensis may express (either naturally or by genetic modification) one or more enzymes which can convert carboxylic acids to amines.

In some embodiments, the derivative of Halomonas rowanensis expresses a carboxylic acid reductase (CAR) and a transaminase (TA). In some embodiments, the derivative of Halomonas rowanensis is genetically modified to express a heterologous CAR and/or a heterologous TA.

In some embodiments, the CAR comprises an amino acid sequence with at least 40% sequence identity to SEQ ID NO:43, 44, 45, 46, 47, or 48. In some embodiments, the CAR comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:43, 44, 45, 46, 47, or 48. In some embodiments, the CAR is a wild type or mutant CAR derived from Nocardia iowensis.

In some embodiments, the TA is an omega-TA (ω-TA). In some embodiments, the ω-TA comprises an amino acid sequence with at least 40% sequence identity to SEQ ID NO:49, 50, 51, 52, 53, or 54. In some embodiments, the ω-TA comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:49, 50, 51, 52, 53, or 54. In some embodiments, the ω-TA is a wild type or mutant ω-TA derived from Chromobacterium violaceum or from Vibrio fluvialis.

In some embodiments, the derivative of Halomonas rowanensis expresses a a phosphopantetheinyl transferase (PPTase) and/or an alanine dehydrogenase (AlaDH). In some embodiments, the derivative of Halomonas rowanensis that expresses the CAR and TA also expresses the PPTase. In some embodiments, the derivative of Halomonas rowanensis that expresses the CAR and TA also expresses the AlaDH. In some embodiments, the derivative of Halomonas rowanensis that expresses the CAR and TA also expresses the PPTase and the AlaDH.

In some embodiments, the PPTase comprises an amino acid sequence with at least 40% sequence identity to the amino acid sequence encoded by SEQ ID NO:42. In some embodiments, the PPTase comprises, or consists, of an amino acid sequence having at least 45%, 50%, 55%, 60%, 65%, 70%, 75%, 80%, 85%, 90%, 95%, 99%, or 100% sequence identity to the amino acid sequence encoded by SEQ ID NO:42.

In some embodiments, the AlaDH comprises an amino acid sequence with at least 40% sequence identity to SEQ ID NO:55. In some embodiments, the AlaDH comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:55.

In some embodiments, the culture is provided with carbon dioxide in addition to atmospheric carbon dioxide. In some embodiments, the culture is provided with carbon dioxide as an alternative to atmospheric carbon dioxide.

In some embodiments, Halomonas is cultured in an environment having a carbon dioxide concentration of at least one of 300, 350, 400, 450, 500, 550, 600, 650, 700, 750, 800, 850, 900, 950, 1000, 1050, 1100, 1150, or 1200 ppm.

In some embodiments, gaseous CO₂ is provided to the culture at a volume concentration at least one of 0.1%, 0.5.%, 1%, 1.5%, 2%, 2.5%, 3%, 4%, 5%, 6%, 7%, 8%, 9%, 10%, 11%, 12%, 13%, 14%, 15%, 16%, 17%, 18%, 19%, or 20% volume/volume (v/v).

In some embodiments, the Halomonas is cultured in a media comprising polluted water. In some embodiments, the polluted water comprises carbon and/or sulfur containing compounds. In some embodiments, the Halomonas cell reduces the concentration of the carbon and/or sulfur containing compounds in the polluted water.

In some embodiments, the Halomonas is cultured in a media comprising at least 10% polluted water. In some embodiments, the Halomonas is cultured in a media comprising one of at least 10%, 15%, 20%, 25%, 30%, 35%, 40%, 45%, 50%, 55%, 60%, 65%, 70%, 75%, 85%, 90%, or 95% polluted water. In some embodiments, the Halomonas is cultured in polluted water.

In some embodiments, the polluted water contains one of at least 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 grams of sulfur containing compounds per litre (g/L).

In some embodiments, the polluted water contains one of at least 0.5, 1, 2, 3, 4, 5, 6, 7, 8, 9, 10, 11, 12, 13, 14, 15, 16, 17, 18, 19, 20, 25, 30, 35, 40, 45, or 50 grams of thiosulfate (Na₂S₂O₃) per litre (g/L).

In some embodiments, the polluted water contains carbon dioxide at a concentration of at least one of 300, 350, 400, 450, 500, 550, 600, 650, 700, 750, 800, 850, 900, 950, 1000, 1050, 1100, 1150, or 1200 ppm.

In another aspect, a method of bioremediation is provided. In some embodiments, the method comprises culturing a Halomonas cell in the presence of carbon dioxide, wherein the Halomonas cell is capable of fixing carbon dioxide. In some embodiments, the Halomonas is cultured in polluted water. In some embodiments, the polluted water comprises carbon and/or sulfur containing compounds and the Halomonas cell reduces the concentration of the carbon and/or sulfur containing compounds in the polluted water.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures:

FIG. 1. Plasmid map for two propane production constructs in Halomonas strains. The CvFAP_{G462V} promoter region differs between the two plasmids by the sequence of the constitutive promoter (P7 or P102)

FIG. 2. Tolerance of Halomonas isolates for salinity, pH and butyric acid. Isolates were cultivated on LB-based medium with variations in the A) pH (7-9), NaCl concentration (6-12%) and B) butyric acid (0-80 mM). Cultures were grown at 37° C. for 16 h. The growth images in A) and B) show the growth screen of Halomonas rowanensis varying pH, salinity and butyrate concentration C) Data is expressed as the minimum, optimal and maximum conditions of pH/salinity where significant growth was observed. The butyrate concentrations indicate the maximum levels tolerated before growth was retarded. H. rowanensis is also known as Halomonas isolate 15.

FIG. 3. Partial 16S rDNA sequence analysis of brine spring isolates.

FIG. 4. Phylogenetic tree of Halomonas isolates. Insets: light microscope images of H. rowanensis at 100× magnification. Strains: H. saliphila st. LCB169; H. flava st. YIM 94343; H. socia st. NY-011 16S; H. subglaciescola st. DSM 4683; H. halmophila st. ATCC 19717; H. elongata st. 1H9; H. alkaliphilia st. 18bAG and H. taeanensis st. BH539.

FIG. 5. Heterotrophic ectoine production of H. rowanensis. A) Halomonas metabolic pathway to ectoine production. Gene annotations from H. rowanensis are shown. B) Ectoine production of H. rowanensis (right-hand bar) and H. bluephagenesis TD01 (left-hand bar) in variable levels of salinity. Cultures were harvested and milked for ectoine with ultrapure water as described previously (Sauer and Galinski, 1998). Error bars represent one standard deviation of triplicate analyses. Enzymes: asd=aspartate semialdehyde dehydrogenase 1; EctA=L-2,4-diaminobutyric acid acetyltransferase; ectB=L-2,4-diaminobutyric acid transaminase; ectC=L-ectoine synthase; ectD=ectoine dioxygenase; lysC=aspartate kinase.

FIG. 6. Growth of H. rowanensis in mineral-based media using polluted water with and without exogenous carbon sources. Growth of H. rowanensis in environmental water samples from the Greater Manchester region (UK) in the A) absence or B) presence of a supplemental carbon source. C) Growth of H. rowanensis in sea water (Irish sea). Cultures were grown for up to 120 h at 30° C. in sanitised and filtered polluted water containing additional minerals/NaCl required for halophilic growth with or without 0.5% (w/v) glycerol. An average of 3 independent biological repeats were performed in part C) with error bars representing 1 standard deviation of the data. The Greater Manchester water samples were obtained from the Medlock and Mersey rivers and the Rochdale canal (UK).

FIG. 7. Chemoautotrophic growth of H. rowanensis. A) Chemoautotrophic growth of three Halomonas species. Cultures were grown in thiosulfate growth medium at 30° C. for 48 h. B) Growth of H. rowanensis with or without supplemental inorganic carbon. Cultures were grown in thiosulfate medium in the presence and absence of 150 mM NaHCO₃ for 100 h at 30° C. C) Radiolabelled carbon fixation of non-permeabilised cultures. Cultures were grown in species-specific growth medium and the ¹⁴C (H¹⁴CO₃⁻) fixation assay was performed as described previously (Sun et al., 2016) with the culture at OD 600 nm=4.0. D) Time course of ¹⁴C labelled bicarbonate fixation in six microorganisms. Cells (OD 600 nm=4.0) were permeabilised with alkyltrimethylammonium bromide with NaH¹⁴CO₃ addition to allow the measurement of total enzymatic carbon fixation activity independently of bicarbonate uptake systems. The H. bluephagenesis strain for parts C) and D) was TD01.

FIG. 8. Putative carbon fixation pathway in H. rowanensis. Enzymes: 1=phosphoenolpyruvate synthase; 2=phosphoenolpyruvate carboxylase; 3=malate dehydrogenase; 4=fumarate hydratase; 5=succinate dehydrogenase; 6=succinyl-CoA ligase; 6a=γ-butyrobetaine,2-oxoglutarate dioxygenase; 7=2-oxoglutarate synthase; 8=isocitrate dehydrogenase [NADP]; 9-10=aconitate hydratase; 11=citrate lyase and 12=NADP-dependent malic enzyme. Metabolites: PEP=phosphoenolpyruvate; αKG=α-ketoglutarate; 3-OHTMAB=3-hydroxy-4-trimethylammoniobutanoate and 4-TMAB=4-trimethylammoniobutanoate. Gene annotation was performed using a combined approach with Rapid Annotations using Subsystems Technology toolkit (RASTtk) along with BLASTp validation, using the KEGG carbon fixation pathways as guide for enzyme discovery.

FIG. 9. Sulfur oxidation systems for energy generation. A) Sox system for thiosulfate oxidation to sulphate in Halothiobacillus. Eight electrons per thiosulfate enter the quinone pool. B) Thiosulfate dehydrogenase (tsaD) c-type cytochrome for oxidation of thiosulfate to tetrathionate.

FIG. 10. Putative thiosulfate and other oxidised sulfur utilisation pathways in H. rowanensis. APS=adenosine-5′-phosphosulfate; PAPS=3′-phosphoadenylyl sulfate; Enzymes: CGS1=cystathionine γ-synthase; CysS=cysteine synthase; fccB=flavocytochrome c; rhdA/glpE/cysA1=thiosulfate sulfur transferases; SAT=sulfate adenylyltransferase; sir/cysJ=sulfite reductase.

FIG. 11. Propane production of H. bluephagenesis TQ10 and/or H. rowanensis expressing plasmid borne CvFAP_{G462V} with a A) weak p7 and B) strong p102 constitutive promoter. In part A, cultures (50 mL) were cultivated in LB60 medium (left bar) or thiosulfate medium (right bar) for 6 h from a 1% inoculum at 30° C. and 200 rpm. Triplicate aliquots (1 mL) were sealed in 4 mL glass vials and incubated overnight at 30° C. and 180 rpm under a blue LED panel. For part B, H. rowanensis was cultivated as above in the two medium types with or without supplemental butyric acid (10 mM). Propane was quantified by Micro GC analysis of manual headspace samples.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

Inventors screened environmental samples obtained from a brine spring in Cheshire, UK for novel bacterial species capable of growth on inexpensive feedstock but optimised for growth at neutral pH for carbon compound production. A novel Halomonas strain was identified along with other strains of interest. Inventors surprisingly found that the newly isolated strain was capable of chemoautotrophic growth. Additionally, inventors assessed whether the newly identified strains could be genetically modified for biofuel production and near neutral carbon biomanufacturing routes.

### Halomonas

Halophilic bacteria have the potential to be extremely useful in biotechnology. These are capable of growing in open non-sterile conditions. As these strains are salt tolerant, they will not be outcompeted so long as there is a high salt content. Furthermore, the addition of a high salt buffer (e.g. at least a 3% salt solution) can be used to control competing bacteria. Halophilic bacteria include those of the genus

### Halomonas.

Exemplary species of Halomonas have been described, including H. rowanensis, H. alimentaria, H. alkaliantarctica, H. alkaliphila, H. almeriensis, H. andesensis, H. anticariensis, H. aquamarina, H. arcis, H. axialensis, H. beimenensis, H. bluephagenesis, H. boliviensis, H. campaniensis, H. campisalis, H. caseinilytica, H. cerina, H. cibimaris, H. cupida, H. daqiaonensis, H. daqingensis, H. denitrificans, H. desiderata, H. elongata, H. eurihalina, H. flava, H. fontilapidosi, H. garicola, H. gomseomensis, H. gudaonensis, H. halmophila, H. halocynthiae, H. halodenitrificans, halophila, H. hamiltonii, H. heilongjiangensis, H. huangheensis, H. hydrothermalis, H. ilicicola, H. janggokensis, H. jeotgali, H. johnsoniae, H. kenyensis, H. koreensis, H. korlensis, H. kribbensis, H. lutea, H. lutescence, H. magadiensis, H. maura, H. meridian, H. mongoliensis, H. muralis, H. nanhaiensis, H. neptunia, H. nitroreducens, H. olivaria, H. organivorans, H. pacifica, H. pantelleriensis, H. qiaohouensis, H. qijiaojingensis, H. ramblicola, H. rifensis, H. sabkhae, H. saccharevitans, H. salicampi, H. salifodinae, H. salina, H. sediminicola, H. shengliensis, H. sinaiensis, H. smyrnensis, H. songnenensis, H. stenophila, H. stevensii, H. subglaciescola, H. subterranean, H. sulfidaeris, H. taeanensis, H. titanicae, H. urumqiensis, H. variabilis, H. ventosae, H. venusta, H. vilamensis, H. xianhensis, H. xinjiangensis, H. zhangjiangensis, and H. zincidurans.

The term Halomonas relates to the genus of Halomonas, and therefore encompasses all Halomonas species and strains, such as Halomonas rowanensis deposited with European Collection of Authenticated Cell Cultures on 10 Feb. 2023 under accession number 23021001, as well as derivatives, mutants, or variants thereof.

Halomonas rowanensis

Halomonas rowanensis deposited with the deposited with European Collection of Authenticated Cell Cultures (ECACC) on 10 Feb. 2023 under accession number 23021001 in accordance with the provisions of the Budapest Treaty on the International Recognition of the Deposit of Microorganisms for the Purposes of Patent Procedure (herein referred to as the ‘Budapest Treaty’).

The inventors identified and characterised Halomonas rowanensis for the first time.

As shown in the examples, H. rowanensis is a chemoautotrophic Halomonas species. It has been shown that H. rowanensis is capable of fixing carbon dioxide, using the fixed carbon dioxide as a carbon source, and metabolically converting the fixed carbon to another carbon containing compound.

It has also been shown that H. rowanensis can be genetically modified through the use of existing synthetic biology constructs that are tailored for other Halomonas species (Amer et al., 2020a. Biotechnol. Biofuels 13, 125; Amer et al., 2020b. Energy Environ. Sci. 13, 1818-1831; Trisrivirat et al., 2020, https://doi.org/10.1093/synbio/ysaa022). It has been shown that H. rowanensis can be genetically modified for chemoautotropic carbon compound production (e.g. propane production) from CO₂.

Derivatives of Halomonas rowanensis

In some embodiments, the organism is a derivative of the Halomonas rowanensis deposited with European Collection of Authenticated Cell Cultures on 10 Feb. 2023 under accession number 23021001.

The derivative of the Halomonas rowanensis deposited with European Collection of Authenticated Cell Cultures on 10 Feb. 2023 under accession number 23021001 may be described as a derivative of Halomonas rowanensis.

A derivative of Halomonas rowanensis may alternatively be described as a mutant or a variant of Halomonas rowanensis.

In some embodiments, the organism is a mutant of the Halomonas rowanensis deposited with European Collection of Authenticated Cell Cultures on 10 Feb. 2023 under accession number 23021001. In some embodiments, the organism is a variant of the Halomonas rowanensis deposited with European Collection of Authenticated Cell Cultures on 10 Feb. 2023 under accession number 23021001.

In some embodiments, the derivative of Halomonas rowanensis expresses one or more heterologous genes. In some embodiments, the derivative of Halomonas rowanensis expresses 1, 2, 3, 4, 5, 6, 7, 8, 9, 10, 11, 12, 13, 14, or 15 heterologous genes.

In some embodiments, the derivative of Halomonas rowanensis expresses one or more heterologous genes for the production of a carbon compound. In some embodiments, the derivative of Halomonas rowanensis expresses 1, 2, 3, 4, 5, 6, 7, 8, 9, 10, 11, 12, 13, 14, or 15 heterologous genes for the production of a carbon compound.

In some embodiments, the derivative of Halomonas rowanensis expresses one or more heterologous genes for C2-C17 carbon compound production. In some embodiments, the Halomonas expresses a heterologous pathway for C2-C17 carbon compound production. In some embodiments the derivative of Halomonas rowanensis expresses one or more heterologous genes for propane and/or linalool production. In some embodiments the derivative of Halomonas rowanensis expresses a heterologous pathway for propane and/or linalool production.

In some embodiments, the derivative of Halomonas rowanensis is modified with one or more heterologous gene for C2-C17 carbon compound production. In some embodiments, the derivative of Halomonas rowanensis is modified with heterologous pathway for C2-C17 carbon compound production. In some embodiments the derivative of Halomonas rowanensis is modified with a heterologous pathway for propane and/or linalool production.

In some embodiments, the derivative of Halomonas rowanensis cell expresses a heterologous fatty acid photodecarboxylase. In some embodiments, the derivative of Halomonas rowanensis cell expresses a fatty acid photodecarboxylase from Chlorella variabilis NC64A (known as CvFAP or CvPAS). In some embodiments, the derivative of Halomonas rowanensis is modified to express a heterologous fatty acid photodecarboxylase. In some embodiments, the derivative of Halomonas rowanensis is modified to express photoalkane synthase from Chlorella variabilis NC64A (known as CvFAP or CvPAS). In some embodiments, the fatty acid photodecarboxylase comprises, or consists, of an amino acid sequence having at least 70%, 75%, 80%, 85%, 90%, 95%, 99%, or 100% sequence identity to SEQ ID NO:1.

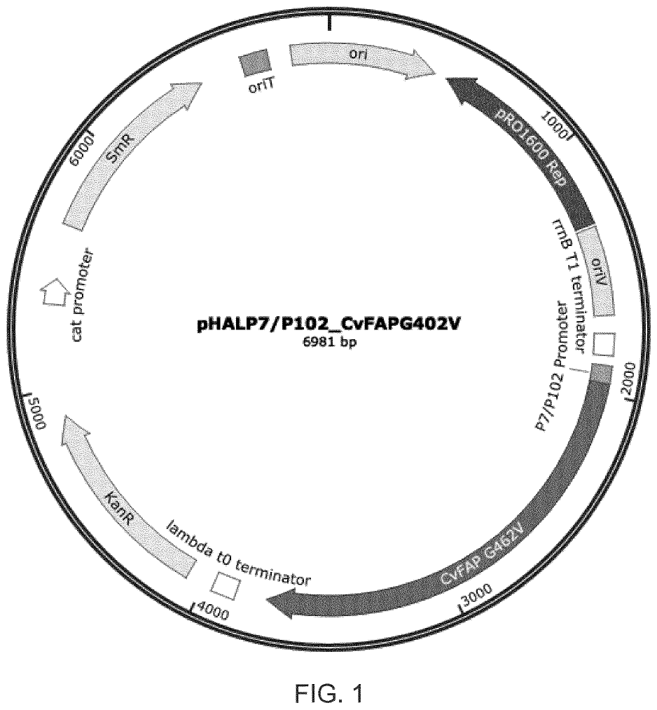

In some embodiments, the derivative of Halomonas rowanensis has been transformed with a pHALP7_CvFAP_{G462V} and/or a pHAPL102_CvFAP_{G462V} plasmid. Both pHALP7-CvFAP_{G462V} and pHALP102-CvFAP_{G462V} are plasmids for propane production and are shown in detail in FIG. 1.

The pHALP7_CvFAP_{G462V} plasmid contains a gene encoding fatty acid photodecarboxylase (CvFAP) from Chlorella variabilis NC64A variant G462V (Amer et al., 2020b). The plasmid also contains a broad-host-range origin of replication (oriV) from Pseudomonas aeruginosa, as well as an origin of transfer (oriT) and a P7 promoter (Trisrivirat et al., 2020, https://doi.org/10.1093/synbio/ysaa022). The plasmid map for pHALP7_CvFAPG_{462V} is shown in FIG. 1.

The pHALP12_CvFAPG_{462V} plasmid contains a gene encoding fatty acid photodecarboxylase (CvFAP) from Chlorella variabilis NC64A variant G462V (Amer et al., 2020b). The plasmid also contains a broad-host-range origin of replication (oriV) from Pseudomonas aeruginosa, as well as an origin of transfer (oriT) and a P12 promoter (Trisrivirat et al., 2020, https://doi.org/10.1093/synbio/ysaa022). The plasmid map for pHALP12_CvFAP G462v is shown in FIG. 1.

In some embodiments, the derivative of Halomonas rowanensis has been transformed with a pHALP7_CvFAP_{G462I} and/or a pHAPL102_CvFAP_{G462I} plasmid.

The pHALP7_CvFAP_{G462I} plasmid contains a gene encoding fatty acid photodecarboxylase (CvFAP) from Chlorella variabilis NC64A variant G462I (Amer et al., 2020b). The plasmid also contains a broad-host-range origin of replication (oriV) from Pseudomonas aeruginosa, as well as an origin of transfer (oriT) and a P7 promoter (Trisrivirat et al., 2020, https://doi.org/10.1093/synbio/ysaa022).

The pHALP12_CvFAP_{G462I} plasmid contains a gene encoding fatty acid photodecarboxylase (CvFAP) from Chlorella variabilis NC64A variant G462I (Amer et al., 2020b). The plasmid also contains a broad-host-range origin of replication (oriV) from Pseudomonas aeruginosa, as well as an origin of transfer (oriT) and a P12 promoter (Trisrivirat et al., 2020, https://doi.org/10.1093/synbio/ysaa022).

In some embodiments, the derivative of Halomonas rowanensis cell expresses a heterologous aldehyde dehydrogenase. In some embodiments, the derivative of Halomonas rowanensis is modified to express a heterologous aldehyde dehydrogenase. In some embodiments, the derivative of Halomonas rowanensis is modified to express Clostridium acetobutylicum ATCC 824 enzyme AdhE2 and/or Clostridium beijerinckii BALDH. In some embodiments, the aldehyde dehydrogenase comprises, or consists, of an amino acid sequence having at least 70%, 75%, 80%, 85%, 90%, 95%, 99%, or 100% sequence identity to SEQ ID NO: 16.

In some embodiments, the derivative of Halomonas rowanensis cell expresses a heterologous aldehyde deformylating oxygenase (ADO). In some embodiments, the derivative of Halomonas rowanensis is modified to express a heterologous aldehyde deformylating oxygenase. In some embodiments, the derivative of Halomonas rowanensis is modified to express aldehyde deformylating oxygenase from Procholorococcus marinus st. MIT9313 (ADO). In some embodiments, the aldehyde deformylating oxygenase comprises, or consists, of an amino acid sequence having at least 70%, 75%, 80%, 85%, 90%, 95%, 99%, or 100% sequence identity to SEQ ID NO: 23.

In some embodiments, the derivative of Halomonas rowanensis is modified to express a variant of the aldehyde deformylating oxygenase from Procholorococcus marinus st. MIT9313 (ADO). In some embodiments, the variant of the aldehyde deformylating oxygenase from Procholorococcus marinus st. MIT9313 (ADO) comprises a substitution at position A134. In some embodiments, the variant of the aldehyde deformylating oxygenase from Procholorococcus marinus st. MIT9313 (ADO) comprises an A134F substitution. In some embodiments, the variant of the aldehyde deformylating oxygenase from Procholorococcus marinus st. MIT9313 (ADO) comprises an amino acid sequence having at least 70%, 75%, 80%, 85%, 90%, 95%, or 99%, sequence identity to SEQ ID NO: 23.

In some embodiments, the derivative of Halomonas rowanensis expresses a heterologous ferredoxin. In some embodiments, the derivative of Halomonas rowanensis is modified to express a heterologous ferredoxin. In some embodiments, the derivative of Halomonas rowanensis is modified to express ferredoxin from Synechocystis sp. PCC6803 (PetF). In some embodiments, the ferredoxin comprises, or consists, of an amino acid sequence having at least 70%, 75%, 80%, 85%, 90%, 95%, 99%, or 100% sequence identity to SEQ ID NO:38.

In some embodiments, the derivative of Halomonas rowanensis cell expresses a heterologous linalool synthase. In some embodiments, the derivative of Halomonas rowanensis is modified to express a heterologous linalool synthase. In some embodiments, the derivative of Halomonas rowanensis is modified to express a heterologous linalool synthase from Streptomyces clavuligerus. In some embodiments, the linalool synthase comprises, or consists, of an amino acid sequence having at least 70%, 75%, 80%, 85%, 90%, 95%, 99%, or 100% sequence identity to SEQ ID NO: 30, SEQ ID NO: 31, SEQ ID NO: 32, and/or SEQ ID NO: 33.

In some embodiments, the derivative of Halomonas rowanensis may express (either naturally or by genetic modification) one or more enzymes which can convert acetyl-CoA into isopentenyl pyrophosphate (IPP), for example by way of the mevalonate-dependent (MVA) pathway. In embodiments, the one or more enzymes may include, for example, acetoacetyl-CoA thiolase (AtoB, EC 2.3.1.9) (from Escherichia coli for example), hydroxymethylglutaryl-CoA synthase (HMGS, EC 2.3.3.10) (from Saccharomyces cerevisiae for example), hydroxymethylglutaryl-CoA reductase (HMGR, EC 1.1.1.34) (from Saccharomyces cerevisiae for example), mevalonate kinase (MK, EC 2.7.1.36) (from Saccharomyces cerevisiae for example), phosphomevalonate kinase (PMK, EC 2.7.4.2) (from Saccharomyces cerevisiae for example), phosphomevalonate decarboxylase (PMD, EC 4.1.1.33) (from Saccharomyces cerevisiae for example) and/or isopentenyl diphosphate isomerase (idi, EC 5.3.3.2) (from Escherichia coli for example).

In some embodiments, the derivative of Halomonas rowanensis may express (either naturally or by genetic modification) one or more enzymes which can convert pyruvate into dimethylallyl pyrophosphate (DMAPP), for example by way of the methylerythritol 4-phosphate (MEP) pathway. In some embodiments, the one or more enzymes may include, for example, 1-deoxyxylulose-5-phosphate synthase (DXS, EC 2.2.1.7), 1-deoxyxylulose-5-phosphate reductoisomerase (DXR, IspC, EC 1.1.1.267), 2-C-methyl-D-erythritol 4-phosphate cytidylyltransferase (YgbP, IspD, EC 2.7.7.60), 4-(cytidine 5′-diphospho)-2-C-methyl-D-erythritol kinase (YchB, IspE, EC 2.7.1.148), (E)-4-hydroxy-3-methylbut-2-enyl-diphosphate synthase (GcpE, IspG, EC 1.17.7.1) and/or 4-hydroxy-3-methylbut-2-en-1-yl diphosphate reductase (LytB, IspH, EC 1.17.7.4). In embodiments, such enzymes may be from E. coli, for example.

In some embodiments, the derivative of Halomonas rowanensis may express (either naturally or by genetic modification) one or more enzymes which can convert isopentenyl pyrophosphate (IPP) and dimethylallyl diphosphate (DMAPP) into geranyl pyrophosphate (GPP). In some embodiments, the derivative of Halomonas rowanensis expresses a heterologous geranyl diphosphate synthase (GPPS). In some embodiments, the derivative of Halomonas rowanensis expresses a heterologous geranyl diphosphate synthase (GPPS) from Abies grandis. In some embodiments, the GPPS comprises, or consists, of an amino acid sequence having at least 70%, 75%, 80%, 85%, 90%, 95%, 99%, or 100% sequence identity to SEQ ID NO: 39 and/or SEQ ID NO: 40.

In some embodiments, the derivative of Halomonas rowanensis may express (either naturally or by genetic modification) one or more enzymes which can convert carboxylic acids to and/or conjugate bases of carboxylic acids (carboxylates) amines.

In some embodiments, the derivative of Halomonas rowanensis expresses a carboxylic acid reductase (CAR) and a transaminase (TA). In some embodiments, the derivative of Halomonas rowanensis is genetically modified to express a heterologous CAR and/or a heterologous TA.

In some embodiments, the CAR comprises an amino acid sequence with at least 40% sequence identity to SEQ ID NO:43, 44, 45, 46, 47, or 48. In some embodiments, the CAR comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:43, 44, 45, 46, 47, or 48. In some embodiments, the CAR is a wild type or mutant CAR derived from Nocardia iowensis.

In some embodiments, the TA is an omega-TA (ω-TA). In some embodiments, the ω-TA comprises an amino acid sequence with at least 40% sequence identity to SEQ ID NO:49, 50, 51, 52, 53, or 54. In some embodiments, the ω-TA comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:49, 50, 51, 52, 53, or 54. In some embodiments, the ω-TA is a wild type or mutant ω-TA derived from Chromobacterium violaceum or from Vibrio fluvialis.

In some embodiments, the derivative of Halomonas rowanensis expresses a a phosphopantetheinyl transferase (PPTase) and/or an alanine dehydrogenase (AlaDH). In some embodiments, the derivative of Halomonas rowanensis that expresses the CAR and TA also expresses the PPTase. In some embodiments, the derivative of Halomonas rowanensis that expresses the CAR and TA also expresses the AlaDH. In some embodiments, the derivative of Halomonas rowanensis that expresses the CAR and TA also expresses the PPTase and the AlaDH.

In some embodiments, the Sfp comprises an amino acid sequence with at least 40% sequence identity to the amino acid sequence encoded by SEQ ID NO:42. In some embodiments, the Sfp comprises, or consists, of an amino acid sequence having at least 45%, 50%, 55%, 60%, 65%, 70%, 75%, 80%, 85%, 90%, 95%, 99%, or 100% sequence identity to the amino acid sequence encoded by SEQ ID NO:42.

In some embodiments, the AlaDH comprises an amino acid sequence with at least 40% sequence identity to SEQ ID NO:55. In some embodiments, the AlaDH comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NOs: 55.

In some embodiments, the derivative of Halomonas rowanensis has been modified to inhibit aldehyde reduction. In some embodiments, the organism has been modified to reduce or eliminate the activity or expression of one or more aldo-keto reductases (AKRs) and/or alcohol dehydrogenases (ADHs).

In some embodiments, the derivative of Halomonas rowanensis comprises a mutation. In some embodiments, the derivative of Halomonas rowanensis comprises more than one mutation. In some embodiments, the derivative of Halomonas rowanensis comprises 1, 2, 3, 4, 5, 6, 7, 8, 9, 10, or more than 10 mutations. In some embodiments, the mutation is a point mutation. In some embodiments, the mutation is a substitution mutation. In some embodiments, the mutation is a deletion. In some embodiments, the derivative of Halomonas rowanensis comprises a point mutation, a substitution mutation, and/or a deletion.

### Methods of Microbial Culture

Microbial cultures, or cultures or microbiological cultures, generally comprise a culture vessel, a growth medium, and at least one microbial cell. In some embodiments, the microbial culture is a lab-scale culture. In some embodiments, the microbial culture is an industrial-scale culture. In some embodiments of the present disclosure, the microbial cell is a Halomonas cell, such as a Halomonas rowanensis cell, or a derivative of a Halomonas rowanensis cell.

A microbial medium, microbial growth medium or microbial culture medium is a liquid, semi-solid or solid designed to support the growth and proliferation of microbial cells. Microbial culture media are well known by scientists in the area of cell culture. Microbial cell culture media types and methods are comprehensively reviewed in A Rouf, Varsha Kanojia, H R Naik, Bazilla Naseer and Tahiya Qadri (2017) An overview of microbial cell culture, Journal of Pharmacognosy and Phytochemistry, Vol. 6, Issue 6 p 1923-1928, the contents of which are incorporated by reference.

Microbial culture media may comprise an appropriate source of energy and compounds which regulate the cell cycle. In addition to nutrients, the medium also helps maintain pH and osmolality. However, in some embodiments, there is no glucose and/or no carbon source present in the media.

Microbial culture media may be liquid (i.e. aqueous), semi-solid (i.e. gelatinous), or completely solid. Semi-solid and solid media may contain agar, gellan gum, or other solidification agents. Liquid media generally do not contain solidification agents.

In some embodiments, the microbial culture media is Luria broth (LB), high salt Luria broth (LB60), minimal media, high salt minimal media, or another suitable media known by the skilled person.

Luria broth (LB) contains 5 g/L yeast extract, 10 g/L tryptone and 10 g/L NaCl pH 7.0, and is widely used and well known to the skilled person. High salt Luria broth (LB60) is identical to LB, but has a higher salt content (60 g/L NaCl). Minimal medium is culture medium for microorganisms that contains the minimal necessities for growth—containing only inorganic salts, water, and optionally a carbon source. High salinity minimal medium contains higher salt content than typical minimal medium. An exemplary high salinity minimal medium is filter sterilised sea water with or without a supplemental carbon source

In some embodiments, the microbial culture media is taken directly from the environment, for example from sea water, brackish water, river water, lake water, pond water. In some embodiments the cells are grown directly in the water taken from the environment, in some embodiments the environmental water is processed (e.g. filtered and/or autoclaved) before use as culture media, in some embodiments further additives (e.g. carbon, mineral, and/or amino acids) are added to the water to support growth of the cells.

In some embodiments, the water is polluted with excess carbon and/or excess sulfur containing compounds. The sulfur containing compound may be a sulfate, a thiosulfate, and/or a sulfite.

In the wild, autotrophic microorganisms (e.g. chemoautotrophs) tend to utilise atmospheric carbon dioxide as their carbon source.

In some embodiments of this invention, microorganisms (e.g. Halomonas) utilise atmospheric carbon dioxide as a carbon source. In some embodiments a carbon supply is provided in addition to atmospheric carbon dioxide. Suitable additional carbon supplies may be provided by the addition of sugar, NaHCO₃, KHCO₃, Na₂CO₃, waste glycerol, pre-treated food waste, pre-treated plant waste, pre-treated seaweed, or supplemental gaseous CO₂ to the culture. In some embodiments the microorganism utilises carbon dioxide as a carbon source and also uses another carbon source (e.g. sugar, NaHCO₃, waste glycerol, pre-treated food waste, pre-treated plant waste, or pre-treated seaweed).

The additional carbon may be supplied as a gas, liquid (including a solution in water) or a solid.

The additional carbon supply may be added continuously or at selected times during the culture or growth cycle of the chemoautotrophic microorganism.

In some embodiments, gaseous carbon dioxide (CO₂) is provided to the culture. In some embodiments, atmospheric CO₂ is provided to the culture. In some embodiments, further carbon dioxide is provided in addition to atmospheric carbon dioxide.

Carbon dioxide (CO₂) is produced as a by-product of many industrial processes such as oil and gas production, cement production, iron and steel production, and electricity generation, as well as many others. This carbon dioxide is typically released into the environment through the combustion of fuels. However, it can be captured, separated, isolated and stored. This disclosure provides methods of utilising this waste carbon dioxide in a process of chemoautotrophic carbon fixation producing products of value and industrial application. In some embodiments, the culture is grown in a CO₂ incubator.

In some embodiments, additional carbon dioxide is provided to the atmosphere surrounding the culture. In some embodiments, carbon dioxide is provided directly to the culture media.

Carbon dioxide may be provided at a concentration of about 420 parts per million (ppm) which is a typical average concentration of atmospheric carbon dioxide.

In some embodiments, gaseous CO₂ is provided at one of about 450, 500, 550, 600, 650, 700, 750, 800, 850, 900, 950, 1000, 1050, 1100, 1150, or 1200 ppm.

In some embodiments, gaseous CO₂ is provided at a concentration of at least one of 300, 350, 400, 450, 500, 550, 600, 650, 700, 750, 800, 850, 900, 950, 1000, 1050, 1100, 1150, or 1200 ppm.

In some embodiments, gaseous CO₂ is provided at a maximum concentration of 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or 2000 ppm.

In some embodiments, gaseous CO₂ is provided at a concentration between 300 ppm and 2000 ppm, 350 ppm and 2000 ppm, 400 ppm and 2000 ppm, 450 ppm and 2000 ppm, 500 ppm and 2000 ppm, 550 ppm and 2000 ppm, 600 ppm and 2000 ppm, 650 ppm and 2000 ppm, 700 ppm and 2000 ppm, 750 ppm and 2000 ppm, 800 ppm and 2000 ppm, 850 ppm and 2000 ppm, 900 ppm and 2000 ppm, 950 ppm and 2000 ppm, or 1000 ppm and 2000 ppm.

In some embodiments, gaseous CO₂ is provided at a concentration between 300 ppm and 1500 ppm, 350 ppm and 1500 ppm, 400 ppm and 1500 ppm, 450 ppm and 1500 ppm, 500 ppm and 1500 ppm, 550 ppm and 1500 ppm, 600 ppm and 1500 ppm, 650 ppm and 1500 ppm, 700 ppm and 1500 ppm, 750 ppm and 1500 ppm, 800 ppm and 1500 ppm, 850 ppm and 1500 ppm, 900 ppm and 1500 ppm, 950 ppm and 1500 ppm, or 1000 ppm and 1500 ppm.

In some embodiments, gaseous CO₂ is provided at a concentration between 300 ppm and 1000 ppm, 350 ppm and 1000 ppm, 400 ppm and 1000 ppm, 450 ppm and 1000 ppm, 500 ppm and 1000 ppm, 550 ppm and 1000 ppm, 600 ppm and 1000 ppm, 650 ppm and 1000 ppm, 700 ppm and 1000 ppm, 750 ppm and 1000 ppm, 800 ppm and 1000 ppm, 850 ppm and 1000 ppm, 900 ppm and 1000 ppm, or 950 ppm and 1000 ppm.

In some embodiments, gaseous CO₂ is provided to the culture at a volume concentration of about one of 0.1%, 0.5.%, 1%, 1.5%, 2%, 2.5%, 3%, 4%, 5%, 6%, 7%, 8%, 9%, 10%, 11%, 12%, 13%, 14%, 15%, 16%, 17%, 18%, 19%, or 20% volume/volume (v/v).

In some embodiments, gaseous CO₂ is provided to the culture at a volume concentration of at least one of 0.1%, 0.5.%, 1%, 1.5%, 2%, 2.5%, 3%, 4%, 5%, 6%, 7%, 8%, 9%, 10%, 11%, 12%, 13%, 14%, 15%, 16%, 17%, 18%, 19%, or 20% volume/volume (v/v).

In some embodiments, gaseous CO₂ is provided to the culture at a volume concentration of at most one of 0.1%, 0.5.%, 1%, 1.5%, 2%, 2.5%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% volume/volume (v/v).

In some embodiments, gaseous CO₂ is provided to the culture at a volume concentration of between 0.1% and 100%, 0.1% and 90%, 0.1% and 80%, 0.1% and 70%, 0.1% and 60%, 0.1% and 50%, 0.1% and 40%, 0.1% and 30%, 0.1% and 20%, 0.1% and 10%, 0.1% and 5%, 0.1% and 2.5%, or 0.1% and 1% volume/volume (v/V).

In some embodiments, gaseous CO₂ is provided to the culture at a volume concentration of between 0.5% and 100%, 0.5% and 90%, 0.5% and 80%, 0.5% and 70%, 0.5% and 60%, 0.5% and 50%, 0.5% and 40%, 0.1% and 30%, 0.5% and 20%, 0.5% and 10%, 0.5% and 5%, 0.5% and 2.5%, or 0.5% and 1% volume/volume (v/V).

In some embodiments, gaseous CO₂ is provided to the culture at a volume concentration of between 1% and 100%, 1% and 90%, 1% and 80%, 1% and 70%, 1% and 60%, 1% and 50%, 1% and 40%, 1% and 30%, 1% and 20%, 1% and 10%, 1% and 5%, or 1% and 2.5% volume/volume (v/v).

In some embodiments, gaseous CO₂ is provided to the culture at a volume concentration of between 5% and 100%, 5% and 90%, 5% and 80%, 5% and 70%, 5% and 60%, 5% and 50%, 5% and 40%, 5% and 30%, 5% and 20%, or 5% and 10% volume/volume (v/v).

In some embodiments, NaHCO₃ is provided to the culture. In some embodiments, NaHCO₃ is provided to the culture to supplement CO₂.

In some embodiments, NaHCO₃, or an equimolar mixture of NaHCO₃ with KHCO₃, is provided to the culture at a concentration of about 1, 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, or 200 mM.

In some embodiments, NaHCO₃, or an equimolar mixture of NaHCO₃ with KHCO₃, is provided to the culture at a concentration of at least 1, 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, or 200 mM.

In some embodiments, NaHCO₃, or an equimolar mixture of NaHCO₃ with KHCO₃, is provided to the culture at a concentration of at most 1, 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, or 200 mM.

In some embodiments, NaHCO₃, or an equimolar mixture of NaHCO₃ with KHCO₃, is provided to the culture at a concentration between 1 mM and 10 mM, 1 mM and 20 mM, 1 mM and 30 mM, 1 mM and 40 mM, 1 mM and 50 mM, 1 mM and 60 mM, 1 mM and 70 mM, 1 mM and 80 mM, 1 mM and 90 mM, 1 mM and 100 mM, 1 mM and 110 mM, 1 mM and 120 mM, 1 mM and 130 mM, 1 mM and 140 mM, 1 mM and 150 mM, 1 mM and 160 mM, 1 mM and 170 mM, 1 mM and 180 mM, 1 mM and 190 mM, or 1 mM and 200 mM.

In some embodiments, NaHCO₃, or an equimolar mixture of NaHCO₃ with KHCO₃, is provided to the culture at a concentration between 5 mM and 10 mM, 5 mM and 20 mM, 5 mM and 30 mM, 5 mM and 40 mM, 5 mM and 50 mM, 5 mM and 60 mM, 5 mM and 70 mM, 5 mM and 80 mM, 5 mM and 90 mM, 5 mM and 100 mM, 5 mM and 110 mM, 5 mM and 120 mM, 5 mM and 130 mM, 5 mM and 140 mM, 5 mM and 150 mM, 5 mM and 160 mM, 5 mM and 170 mM, 5 mM and 180 mM, 5 mM and 190 mM, or 5 mM and 200 mM.

Any suitable container or culture vessel may be used to propagate cells according to the methods and compositions described here. In some aspects, the container or culture vessel is a natural body, such as a lake or pond.

The skilled person is aware of many different types of culture vessels. Exemplary culture vessels include: plates, dishes, flasks, bottles, bioreactors, or fermenters. Also known as culture plates, culture dishes, culture flasks, culture bottles, culture bioreactors, or culture fermenters. Culture vessels can be constructed from a number of materials such as metal, glass, plastics and polymers such as polystyrene and polyester.

Culture plates are low flat-bottomed laboratory containers for growing cells on a thin layer of nutrient medium. Plates and dishes can be used with liquid and solid media. Culture dishes are similar to culture plates. The most common types of culture plates and dishes are the Petri dish and the multiwell plate. Multiwell plates are available in many sizes, for example as a 2-well plate, 4-well plate, a 6-well plate, a 12-well plate, a 24-well plate, a 48-well plate, a 96-well plate, or a 384 well plate. Petri dishes and multiwell plates are available from many manufacturers (e.g. Thermo Fisher Scientific) with different specifications. Plates are available in different colours (e.g. clear, black and white), and are usually made from polystyrene.

Culture flasks are generally used with liquid cultures and typically resemble bottles. Flasks are generally constructed from glass, polycarbonate or polystyrene. It is possible to reuse and sterilise glass culture flasks, whereas disposable polycarbonate or polystyrene flasks are widely available (e.g. Thermo Fisher Scientific Nunc EasYFlasks). Culture flasks are available in many sizes, such as T25, T75, T175, T225, T300, T1000 and T2000. Different size flasks have different volumes and different surface areas.

In some embodiments, the culture vessel has a volume of at least 0.5, 0.6, 0.7, 0.8, 0.9, 1.0, 1.5, 2.0, 3.0, 4.0, 5.0, 10.0, 15.0, 20.0, 25.0, 30.0, 35.0, 40.0, 45.0, 50.0, 60.0, 70.0, 80.0, 90.0, 100.0, 125.0, 150.0, 175.0, 200.0, 300.0, 400.0, 500.0, 600.0, 700.0, 800.0, 900.0, or 1000.0 mL. In some embodiments, the culture vessel has a volume of at least 0.5, 0.6, 0.7, 0.8, 0.9, 1.0, 1.5, 2.0, 3.0, 4.0, 5.0, 10.0, 15.0, 20.0, 25.0, 30.0, 35.0, 40.0, 45.0, 50.0, 60.0, 70.0, 80.0, 90.0, 100.0, 125.0, 150.0, 175.0, 200.0, 225.0, or 250.0 litres (L).

A bioreactor may comprise a regulated bioreactor, in which one or more conditions may be controlled or monitored, for example, oxygen partial pressure. Devices for measuring and regulating these conditions are known in the art. For example, oxygen electrodes may be used for oxygen partial pressure and/or monitoring dissolved oxygen concentration (dO₂). The oxygen partial pressure can be regulated via the amount and the composition of the selected gas mixture (e.g., air or a mixture of air and/or oxygen and/or nitrogen and/or carbon dioxide). Suitable devices for measuring and regulating the oxygen partial pressure are described by Bailey, J E. (Bailey, J E., Biochemical Engineering Fundamentals, second edition, McGraw-Hill, Inc. ISBN 0-07-003212-2 Higher Education, (1986)) or Jackson A T. Jackson A T., Verfahrenstechnik in der Biotechnologie, Springer, ISBN 3540561900 (1993)). A bioreactor may comprise a photobioreactor. A photobioreactor is a bioreactor which provides an artificial light source to the cultured organism. For example, the photobioreactor may comprise an integral LED system for the provision of light. A bioreactor may comprise a Continuous Stirred Tank Reactor (CSTR) system. A bioreactor may comprise a flat bed photobioreactor system.

Alternatively, a culture may be static, i.e. where active agitation of the culture/culture media is not employed, optionally wherein mixing is performed via gas bubbling.

### Chemoautotrophs

In some embodiments, the Halomonas is an autotroph. An autotroph, or primary producer, is an organism that produces complex organic compounds (such as carbohydrates, fats, and proteins) using carbon from simple substances such as carbon dioxide.

In some embodiments, the Halomonas is a chemoautotroph. Chemoautotrophs, or chemotrophic autotroph, obtain energy by the oxidation of electron donors in their environments and can fix carbon dioxide and synthesize organic compounds from carbon dioxide.

In some embodiments, Halomonas obtains energy by a reaction with an inorganic compound. In some embodiments, Halomonas obtains energy through the action of an enzyme with an inorganic compound. In some embodiments, Halomonas obtains energy by the reduction, oxidation, or hydrolysis of an inorganic compound. In some embodiments, the inorganic compound is a sulfur containing compound (e.g. thiosulfate, sulfuric acid, sulfur, sulfate, and/or sulfite).

The Halomonas may be a facultative chemoautotroph. A facultative chemoautotroph is a chemoautotroph equally capable of utilizing either inorganic substrate or organic substrate as the energy source for growth. Therefore, a facultative chemoautotroph may use carbon dioxide and/or another carbon source (e.g. sugar, NaHCO₃, Na₂HCO₃, KHCO₃, waste glycerol, pre-treated food waste, pre-treated plant waste, or pre-treated seaweed).

Chemoautotrophs are capable of fixing carbon from their surrounding environment. Therefore, Halomonas may be capable of fixing carbon dioxide.

Carbon fixation, or carbon assimilation, is the process by which inorganic carbon (particularly in the form of carbon dioxide) is converted to organic compounds by living organisms. The compounds are then used to store energy and as structure for other biomolecules. Chemoautotrophs are capable of fixing carbon from their surrounding environment. Therefore, Halomonas may be capable of fixing carbon dioxide.

As used herein, “capable of fixing carbon dioxide” means that an organism is able to convert inorganic carbon (e.g. CO₂) to other carbon compounds (e.g. organic compounds such as isocitrate, citrate, malate, pyruvate, fumarate, acetyl-CoA, succinate, and/or succinyl-CoA).

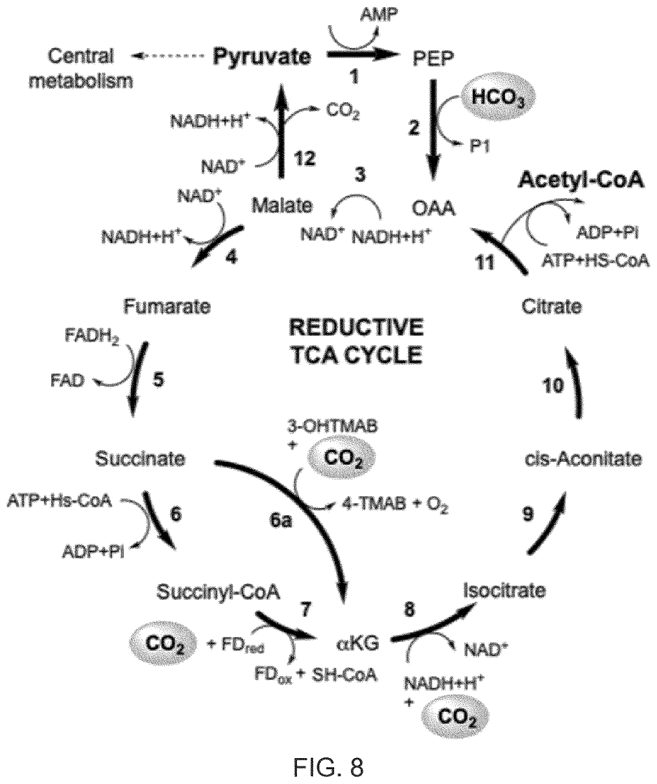

FIG. 8 shows a putative carbon fixation pathway for Halomonas rowanensis. This non-limiting model is provided as an example, and other carbon fixation pathways may be possible. This figure shows that pyruvate can be produced from an inorganic carbon source (CO₂) in a chemoautotroph. Pyruvate is then used in central metabolism in well-understood metabolic pathways to produce other known carbon compounds such as acyl-CoA and fatty acids.

Prior to the current research, only one species of chemoautotrophic Halomonas had been identified (Mishra et al., 2017. Process Biochem. 55, 133-145). However, genetic modification of this organism has not been completed to enable the heterologous expression of an enzyme for the chemoautotrophic production of carbon compounds.

### Methods of Producing Carbon Compounds

Aspects of the current disclosure relate to methods of producing carbon compounds. Methods of producing carbon compounds may comprise culturing a microorganism (e.g. a Halomonas cell) and isolating a carbon compound from the culture.

Any suitable method of culture may be used, for example methods described in previous sections of the description.

The carbon compounds may be a C2-C17 carbon compound, a hydrocarbon, bio-LPG, an alkane, a bioalkane, an alkene, a bioalkene, a C3-C6 alkane, propane, butane, isobutane, ethanol, linalool, an amine, and/or ectoine.

As used herein, a “CX” carbon compound is one having a total carbon number of X. For example, propane is a C3 carbon compound, and linalool is a C10 carbon compound. The “CX” carbon is the carbon at the Xth position. For example, the C5 carbon of stearic acid is the carbon at the 5th position.

As used herein, “isolation of a carbon compound from the culture” means separation from another portion of the culture e.g. the microbial cells and/or the culture medium. However, “isolation of a carbon compound from the culture” does not mean that the carbon compound needs to be removed in a pure form.

In some embodiments, carbon compounds of interest are secreted into the media and can be isolated from the media.

In other embodiments, carbon compounds of interest are not secreted and need to be isolated from the microorganism fraction.

The first step for both of these methods is to separate the microorganism fraction from the liquid fraction. The liquid fraction is the liquid portion (e.g. culture media) which lies above a sediment formed by the solid microorganisms and below the culture headspace (gaseous fraction). The microorganism fraction is the solid sediment fraction which forms below the liquid fraction.

The microorganism fraction can be separated from the liquid fraction in a number of ways, including filtration, chromatography, evaporation, sedimentation, and centrifugation. Following this crude separation, the compounds of interest can be separated from their fraction.

Compounds that are secreted into the media from microorganisms and are present in the liquid fraction can be separated in a number of ways, including chromatography, distillation, crystallisation, and pervaporation. For example, the separation of ethanol from the other components of the media liquid fraction is essentially the separation of ethanol from water with the addition of impurities.

Distillation is the process of separating the components or substances from a liquid mixture by using selective boiling and condensation. The process takes advantage of the fact that different compounds have different boiling points. There are a number of different types of distillation that could be used to separate carbon compounds from media liquid fraction: simple distillation, fractional distillation, vacuum distillation, and azeotropic distillation, to name a few.

In other embodiments, carbon compounds of interest are gaseous and are secreted into the culture headspace from which they can be isolated.

The isolation of secreted gaseous carbon compounds from the culture headspace can be performed continuously throughout the growth of the microorganism. This could be facilitated by a continuous aeration of the culture, with the carbon compound extracted from the continuous stream of exhaust gas outside of the culture vessel. Aeration can be performed by bubbling compressed air, 100% oxygen or a combination of gases into the culture at flow rates from 0.1-2.0 volume of air sparged per unit volume of growth medium per minute (vvm). For example, for a 1 litre culture, 0.5 vvm is equivalent to 0.5 litre air flow per minute.

Other compounds of interest which are not secreted, and need to be isolated from the microorganism fraction, require an additional processing step to isolate the compound. To make the compound available for separation, the cells may need to be lysed. Cells can be lysed through mechanical homogenization, ultrasonic homogenisation, pressure homogenisation, freeze-thaw treatment, heat treatment, osmotic lysis and chemical lysis. Following this, the compounds can be isolated through methods known in the art.

Remaining cell mass from the microorganism fraction can be harvested and used as either fertiliser or in animal feeds, or as feedstock for other biotechnological processes. These products are often high in nutrients, minerals, protein, oil, and/or carbohydrates, and have value as fertiliser or in animal feeds. The remaining cell mass can be either whole cells or the solid fraction of lysed cells.

In some embodiments, the carbon compound is purified. As used herein, “purification of a carbon compound” means that the relative amount of the carbon compound is increased compared to other components of the extract/product i.e. contaminants are removed from the extract/product. The carbon compound does not necessarily have to be 100% pure, although it may be in some embodiments.

In some embodiments, the carbon compound is purified after it is isolated from the culture. In some embodiments, the carbon compound is more than 50% pure after purification. In some embodiments, the carbon compound is more than one of 60%, 70%, 80%, 90%, 91%, 92%, 93%, 94%, 95%, 96%, 97%, 98%, or 99% pure after purification.

### Genetic Modification

The Halomonas cell(s) used in methods according to some aspects of the present disclosure may be genetically modified to express heterologous genes, which may optionally result in additional enzymatic activity. For example, the Halomonas cell(s) may be genetically modified to express a heterologous fatty acid photodecarboxylase.

Halomonas rowanensis cell(s) which are genetically modified to express a heterologous gene may be defined as a derivative of Halomonas rowanensis.

Vectors may be used to introduce heterologous genes into Halomonas. The vector may be an expression vector for expression of the foreign genetic material in the cell. Such vectors may include a promoter and/or a ribosome binding site (RBS) sequence operably linked to the nucleotide sequence encoding the sequence to be expressed. A vector may also include a termination codon and expression enhancers. Such expression vectors are routinely constructed in the art of molecular biology and may for example involve the use of plasmid DNA and appropriate initiators, promoters, RBS, enhancers and other elements, such as for example polyadenylation signals, which may be necessary and which are positioned in the correct orientation in order to allow for recombinant protein expression.

The vector may be used to replicate the nucleic acid in a compatible host cell. Therefore, nucleic acids according to the present invention can be produced by introducing a polynucleotide into a replicable vector, introducing the vector into a compatible host cell and growing the host cell under conditions that bring about replication of the vector.

Vectors may include a promoter sequence operably linked to the nucleotide sequence encoding the gene sequence to be expressed. A vector may also include a termination codon and expression enhancers. Any suitable vectors, promoters, enhancers and termination codons known in the art may be used to express the enzymes from a vector according to the invention. Suitable vectors include plasmids, binary vectors, viral vectors, cosmids, and artificial chromosomes (e.g. yeast artificial chromosomes).

A construct or vector comprising a nucleic acid as described above need not include a promoter or other regulatory sequence, particularly if the vector is to be used to introduce the nucleic acid into cells for recombination into the genome.

Constructs and vectors may further comprise selectable genetic markers consisting of genes that confer selectable phenotypes such as resistance to antibiotics such as kanamycin, hygromycin, phosphinotricin, chlorsulfuron, methotrexate, gentamycin, spectinomycin, chloramphenicol, ampicillin, etc.

Those skilled in the art can construct vectors and design protocols for recombinant gene expression, for example in a microbial cell. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 3^{rd} edition, Sambrook et al, 2001, Cold Spring Harbor Laboratory Press and Protocols in Molecular Biology, Second Edition, Ausubel et al. eds. John Wiley & Sons, 1992.

Any suitable vectors, promoters, enhancers and termination codons known in the art may be used to express a polypeptide from a vector according to the invention. In some embodiments, the vector may be a plasmid, phage, cosmid, MAC, virus, etc.

Other suitable vectors would be apparent to persons skilled in the art. By way of further example in this regard we refer to Sambrook et al., 2001, Molecular Cloning: a laboratory manual, 3^{rd} edition, Cold Harbour Laboratory Press.

The term “operably linked” may include the situation where a selected nucleotide sequence and regulatory nucleotide sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleotide sequence if the regulatory sequence is capable of effecting transcription of the nucleotide sequence. The resulting transcript may then be translated into a desired peptide or polypeptide. The promoter may be a T7-like promoter.

In some embodiments, the vector may comprise an element for facilitating translation of encoded protein from mRNA transcribed from the construct. For example, the construct may comprise a ribosomal binding site (RBS) such as a Shine-Delgarno (SD) sequence upstream of the start codon. In some embodiments, RBS sequences may be designed to provide for different levels of expression of the encoded proteins.

In some embodiments, the vector may encode one or more regulatory elements for modulating expression of the encoded protein(s). In some embodiments, the response element is an element that causes upregulation of gene or protein expression in response to treatment with a particular agent. For example, the agent may induce transcription of DNA encoding the protein(s) from a vector including a response element for the agent. In some embodiments the agent may be isopropyl β-D-1-thiogalactopyranoside (IPTG), and the vector may comprise a lac operator. Other induction agent/response element combinations are known in the art.

In some embodiments, the vector may encode one or more response elements for constitutive expression of the encoded protein(s), such that no induction is necessary.

In some embodiments the vector may comprise a transcription terminator sequence downstream of the sequences encoding to the protein or proteins of interest. In some embodiments the terminator may be a T7 terminator sequence. In some embodiments the vector may comprise a sequence encoding a detectable marker in-frame with the sequence encoding the protein of interest to facilitate detection of expression of the protein, and/or purification or isolation of the protein (e.g. a His, (e.g. 6×His (SEQ ID NO: 56)), Myc, GST, MBP, FLAG, HA, E, or Biotin tag, optionally at the N- or C-terminus).

The nucleic acids/expression vectors can be introduced into a cell by any suitable means, which are well known to the skilled person. In some embodiments the nucleic acids/expression vectors are introduced into a cell by transformation, transduction, conjugation, transfection or electroporation. T7-like promoter systems may be preferred. T7 RNA polymerase is well known in the art. It is a very active enzyme, synthesising RNA at a high rate several times that of E. coli RNA polymerase.

Furthermore, it has a lower frequency of termination, and its transcription can circumnavigate a plasmid, resulting in RNA several times the plasmid length in size. T7 RNA polymerase is also highly selective for initiation at its own promoter sequences and is resistant to antibiotics such as rifampicin that inhibit E. coli RNA polymerase.

“T7-like” promoter systems are IPTG-inducible system that work like the viral polymerase T7 (IPTG-inducible; found in pET system vectors) but is a compatible system in other bacterial species, e.g. Halomonas. The gene MmP1 is a T7-like promoter that enables the IPTG-inducible expression of recombinant proteins in Halomonas when the organism contains a genomic copy of the MMP1 RNA polymerase (Zhao H et al 2017 Novel T7-like expression systems used for Halomonas. Metabolic Engineering 39: p. 128-140 which is herein incorporated by reference in its entirety). Preferably, the Halomonas strain comprises the MmP1 gene, either chromosomally integrated or on a vector or plasmid.

Alternative IPTG-inducible vectors may be preferred. These include non-T7-like promoters P_{tac}, P_{lacUV5} and P_{trc}.

Vectors containing hybrid promoters may be preferred. This may contain native Halomonas Pₚₒᵣᵢₙ or other promoters that have been engineered to convert them to inducible or constitutive promoters. Hybrid vectors may contain randomised variable region sequences that confer different expression levels of recombinant proteins, e.g. constitutive promoters found in vectors pHALP7 and pHALP102.

The pHALP7 and pHALP102 vectors may be preferred. Full details of these vectors can be found in Trisrivirat et al., 2020 (https://doi.org/10.1093/synbio/ysaa022), which is hereby incorporated by reference. FIG. 1 shows plasmid maps as used in the examples for propane production in Halomonas strains. The CvFAPG_{462V} promoter region differs between the two plasmids by the sequence of the constitutive promoter (P7 or P102). The p7 promoter is a weak constitutive promoter, and the p102 promoter is a strong constitutive promoter. Constitutive promoters are defined as promoters active in vivo in all circumstances, and, on the other hand, inducible promoters are switched ON and OFF by transcription factors depending on the in vivo conditions

A vector, or multiple vectors, can be used to introduce one or more heterologous genes into the Halomonas cell(s). In some embodiments, the Halomonas is modified with a heterologous pathway for the production of carbon compounds. For example, the Halomonas may be modified with a heterologous pathway for propane and/or linalool production.

### Hydrocarbons

As used herein, “hydrocarbons” are organic compounds comprising a backbone consisting of hydrogen and carbon. Hydrocarbons include alkanes, alkenes, arenes, cycloalkanes and alkynes. An alkane is a saturated hydrocarbon of the general formula CₙH₂ₙ₊₂. Alkanes herein include straight chained (i.e. unbranched) and branched alkanes. An alkene is an unsaturated hydrocarbon with at least one carbon-carbon double bond. Preferred hydrocarbons are isobutane, butane and propane, or a blend thereof commonly referred to as “liquid petroleum gas (LPG)”. Thus, bio-alkanes are biologically-derived alkanes, bio-alkenes are biologically-derived alkenes, and bio-LPG is a biologically-derived blend of gaseous hydrocarbons commonly found in petroleum-based LPG blends.

Propane is a three-carbon alkane with the molecular formula C₃H₈. It is a gas at standard temperature and pressure, but compressible to a transportable liquid.

Butane or n-butane is an alkane with the formula C₄H₁₀. Butane is a gas at room temperature and atmospheric pressure.

Isobutane, also known as i-butane, 2-methylpropane or methylpropane, is a chemical compound with a molecular formula HC(CH₃)₃. It is an isomer of butane. Isobutane is a colourless, odourless gas. It is the simplest alkane with a tertiary carbon atom.

As used herein, a “CX” carbon compound, hydrocarbon, or fatty acid is one having a total carbon number of X. For example, butane and isobutane are both C4 hydrocarbons/carbon compounds. The “CX” carbon is the carbon at the Xth position. For example, the C5 carbon of stearic acid is the carbon at the 5th position.

As used herein, “chain length” refers to the number of carbons in the longest continuous chain. For example, n-pentane has a chain length of 5, n-butane has a chain length of 4, whilst isobutane and propane have a chain length of 3.

### Hydrocarbon Production

Hydrocarbons, such as bio-alkanes (e.g. propane, butane, and/or isobutane), can be produced in vivo. For example, the method may comprise the steps of:
(1) the chemoautotrophic production of acyl-coenzyme A (acyl-CoA),
(2) conversion of acyl-CoA to a fatty acid using acyl-CoA thioester hydrolase, and
(3) conversion of a fatty acid to an alkene/alkane using a fatty acid photodecarboxylase.

“Fatty acid photodecarboxylase”, as used herein, refers to an enzyme with fatty acid photodecarboxylase activity, i.e. being capable of catalysing the removal of the carboxylic acid group from an n-fatty acid to produce an n-alkane or -alkene, in particular the direct removal without an aldehyde intermediate and without introducing a terminal unsaturation. Fatty acid photodecarboxylase activity can be measured by methods available to the skilled person.

As used herein, “fatty acid” refers to molecules containing a carboxylic acid (—COOH) with an aliphatic hydrocarbon chain. “Fatty acids” include salts and ions of fatty acids. For example, the fatty acid “butyric acid” includes the free acid butyric acid as well as butyrate, etc. “Short-chain” fatty acid as used herein, unless otherwise stated, refers to fatty acids having a 2-8 carbon chain length. Short-chain fatty acids may be 2, 3, 4, 5, 6, 7, or 8 carbons in chain length, for example 2-7, 2-6, 2-5, 2-4, 2-3 or 2 carbons in length. “Long-chain” fatty acids refers to those fatty acids which have longer chains than short-chain fatty acids. For example, long-chain fatty acids may refer to those which have a chain length of 13 or greater, preferably a chain length of 13-21, 13-20, 13-19, 13-18, 13-17, 13-16, 13-15, 13-14, 14-21, 14-20, 14-19, 14-18, 14-17, 14-16, 14-15, 14, 15-21, 15-20, 15-19, 15-18, 15-17, 15-16, 15, 16-21, 16-20, 16-19, 16-18, 16-17, 16, 17-21, 17-20, 17-19, 17-18, 17, 18-21, 18-20, 18-19, 18, 19-21, 19-20, 19, 20-21, 20, or 21 carbons.

Exemplary fatty acid photodecarboxylases are encoded by a polypeptide having the amino acid sequence of one of SEQ ID NOs: 1 to 14. In particular, SEQ ID NO:1 encodes photoalkane synthase from Chlorella variabilis NC64A (CvFAP), which is capable of blue light dependent decarboxylation of free fatty acids to n-alkanes or alkenes. CvFAP is a member of the glucose-methanol-choline oxidoreductase family, contains a bound photoexcitable FAD cofactor with a quantum yield of >80% (0.86+0.13 s-1), and shows a preference for long chain fatty acids, particularly palmitic acid (C16).

The 654 amino acid SEQ ID NO:1 corresponds to the full length polypeptide sequence for CvFAP (UniProt: A0A248QE08).

The native sequence comprises a 61 amino acid chloroplast targeting sequence at the N terminus, which is excised during transport into the chloroplast. This sequence is removed for expression in bacteria (no chloroplasts) and replaced by a methionine residue in the 594 amino acid SEQ ID NO:2:

In this specification, “position G462 of SEQ ID NO:1” also refers to “position G402 of SEQ ID NO:2”. “Position G462 of SEQ ID NO:1” shall be taken as equivalent to and interchangeable with “position G402 of SEQ ID NO:2”.

SEQ ID NOs: 3 to 14 are homologues to SEQ ID NO:1 found in Aureococcus anophagefferens (SEQ ID NO: 3), Chondrus crispus (SEQ ID NO:4), Chrysochromulina spp. (SEQ ID NO:5), Cyanidioschyzon merolae (SEQ ID NO:6), Chlamydomonas reinhardtii (SEQ ID NO:7), Coccomyxa subellipsoidea (SEQ ID NO: 8), Gonium pectorale (SEQ ID NO:9), Phaeodactylum tricornutum (SEQ ID NO:10), Emiliania huxleyi (SEQ ID NOs: 11 and 12), Nannochloropsis gaditana (SEQ ID NO:13), and Volvox carteri f. nagariensis (SEQ ID NO:14). The skilled person will appreciate how to perform sequence alignment to determine which residues are equivalent to G462 of SEQ ID NO:1.

In this specification “fatty acid photodecarboxylase” refers to a fatty acid photodecarboxylase from any species and includes isoforms, fragments, variants or homologues of fatty acid photodecarboxylase from any species. Homologues include orthologues. In some embodiments, the fatty acid photodecarboxylase is a prokaryotic fatty acid photodecarboxylase, e.g. a bacterial fatty acid photodecarboxylase. In some embodiments, the fatty acid photodecarboxylase is from, or is derived from, a microalgae, for example in a species within the genus Volvox, Chlamydomonas, Gonium, Chlorella, Coccomyxa, Aureococcus, Phaeodactylum, Chrysochromulina, Emiliania, Chondrus, or Cyanidioschyzon. Exemplary fatty acid photodecarboxylases are those found in Volvox carteri f. nagariensis, Chlamydomonas reinhardtii, Gonium pectorale, Chlorella variabilis, Coccomyxa subellipsoidea, Aureococcus anophagefferens, Phaeodactylum tricornutum, Chrysochromulina spp., Emiliania huxleyi, Chondrus crispus, or Cyanidioschyzon merolae.

The fatty acid photodecarboxylases provided herein are intended to be variants of a wild type that are not identical in terms of amino acid sequence to a naturally occurring wild type enzyme. As such, they may be described as “mutant”, “non-naturally occurring”, “variant” or “modified”. Any amino acid substitutions that may have the effect of modifying the subject enzyme (e.g. the sequence of SEQ ID NO:1 or SEQ ID NO: 2) so as to create a wild type sequence of a different enzyme, e.g. a homologue such as one of SEQ ID NOs 3-14 (or the mature amino acid sequence thereof lacking the chloroplast targeting sequence) is optionally excluded from the disclosure and may optionally be disclaimed from the invention claimed.

Fragments, variants, isoforms and homologues of a fatty acid photodecarboxylase may optionally be characterised by an ability to catalyse conversion of a fatty acid into an alkane or alkene, in particular of a short-chain fatty acid to a short-chain alkene or alkane.

The fatty acid photodecarboxylases of the various aspects of the invention may be described in terms of similarity to a reference fatty acid photodecarboxylase. For example, the fatty acid photodecarboxylases may comprise at least 40% sequence identity to a reference sequence. The fatty acid photodecarboxylases of the various aspects of the invention may comprise at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% sequence identity to a reference sequence. The reference sequence may be any fatty acid photodecarboxylase, and may comprise an amino acid sequence corresponding to SEQ ID NO:1, SEQ ID NO:2, or any one of SEQ ID NOs: 3 to 14. In preferred embodiments, the reference sequence is selected from SEQ ID NO:1 or 2. The fatty acid photodecarboxylases of the invention may have improved decarboxylase activity and/or give a higher yield compared to a reference fatty acid photodecarboxylase and/or may have a change in the preferred substrate.

The fatty acid photodecarboxylases of the present invention comprise an amino acid substitution at a position corresponding to G462 of SEQ ID NO:1. A steric block to long chain fatty acid binding may be introduced by substituting the relatively small glycine side chain of G462 with a larger side chain. The amino acid substitution at the position corresponding to G462 of SEQ ID NO:1 may be made with any other amino acid not found at that position in the wild type sequence. Included in the disclosure is a method of substituting the amino acid at the position corresponding to G462 of SEQ ID NO:1 with any other amino acid not found at that position in the wild type sequence as specified herein.

Preferably, the substitution replaces the amino acid at the position corresponding to G462 of SEQ ID NO: 1 with an amino acid the side chain of which sterically blocks access to the solvent exposed substrate-binding channel, e.g. so as to physically obstruct the channel so as to prevent binding of a long-chained fatty acid as defined herein. A steric block may be introduced by substituting an amino acid for one with a large, bulky, non-charged and/or non-polar side-chain. For example, a steric block may be introduced by substituting the relatively small glycine side chain (G) of G462 of SEQ ID NO: 1 with a larger, bulkier, non-charged and/or non-polar side chain.

In some embodiments, the amino acid at a position corresponding to G462 of SEQ ID NO:1 is substituted for a bulky residue selected from V, F, I, L, A, Y, C, H, N, Q, and W. In some embodiments, the amino acid at a position corresponding to G462 of SEQ ID NO:1 is substituted for a non-charged residue selected from V, F, I, L, A, Y, C, N, Q, and W. In some embodiments, the amino acid at a position corresponding to G462 of SEQ ID NO:1 is substituted for a non-polar residue selected from V, F, I, L, A, and W.

In some embodiments, the amino acid substitution at a position corresponding to G462 of SEQ ID NO:1 is G462V. In some embodiments, the amino acid substitution is G462I. In some embodiments, the amino acid substitution is G462F. In some embodiments, the amino acid substitution is G462L. In some embodiments, the amino acid substitution is G462A. In some embodiments, the amino acid substitution is G462W. In some embodiments, the amino acid substitution is G462Y. In some embodiments, the amino acid substitution is G462C. In some embodiments, the amino acid substitution is G462H. In some embodiments, the amino acid substitution is G462N. In some embodiments, the amino acid substitution is G462Q. In some embodiments, the amino acid substitution is G462Y.

The skilled person is well able to identify corresponding positions to the indicated positions in fatty acid photodecarboxylases other than that provided by reference sequence SEQ ID NO:1. Corresponding positions can be identified e.g. by alignment of the amino acid sequence of a given fatty acid photodecarboxylase to the amino acid sequence of SEQ ID NO:1. Sequence alignments for such purposes can be achieved in various ways known to a person of skill in the art, for instance, using publicly available computer software such as ClustalOmega (Soding, J. 2005, Bioinformatics 21, 951-960).

In some embodiments, the fatty acid photodecarboxylases further comprise the consensus sequence

(SEQ ID NO: 15)
G-X1-L-X2-X3-X4-X5-C-[D/E]-X6-G-[A/G]-F-X7-[K/R],

X being any amino acid.

Preferably, X1 can be selected from the group consisting of P, L and G. Preferably, X2 can be selected from the group consisting of T and A. Preferably, X3 can be selected from the group consisting of T, S and C. Preferably, X4 can be selected from the group consisting of P, T and A. Preferably, X5 can be selected from the group consisting of G and A. Preferably, X6 can be selected from the group consisting of H, N and R. Preferably, X7 can be a hydrophobic amino acid, especially selected from the group consisting of L, V, A and F.

Other in vivo routes to the production of bio-alkanes are possible, for example through the expression of an aldehyde dehydrogenase, ferredoxin and an aldehyde deformylating oxygenase. This route to the production of bio-alkanes could comprise catalysis of the conversion of butyryl-CoA to butyraldehyde using an aldehyde dehydrogenase, followed by catalysis of the conversion of butyraldehyde to propane using an aldehyde deformylating oxygenase activated by electron transfer from reduced ferredoxin.

As used herein, “aldehyde dehydrogenase” refers to an enzyme capable of catalysing the conversion of acyl-CoA to aldehydes, for example the conversion of butyryl-CoA to butyraldehyde.

Some aldehyde dehydrogenases, such as the Clostridium acetobutylicum ATCC 824 enzyme AdhE2 (aldehyde/alcohol dehydrogenase; GenBank ID: Q9ANR5), are bi-functional enzymes that, in addition to converting butyrl-CoA to butyric acid, also convert butyryl-CoA to butanol, which is a toxic and undesirable by-product. Consequently, in some embodiments, an aldehyde dehydrogenase as employed by the invention does not catalyse an alcohol-forming reaction.

An exemplary aldehyde dehydrogenase is that from Clostridium beijerinckii (BALDH), the amino acid sequence of which is provided by SEQ ID NO:16. Other aldehyde dehydrogenases comprise polypeptides comprising an amino acid sequence selected from SEQ ID NO:17-22. In some embodiments, the butyraldehyde dehydrogenase comprises, or consists of, the amino acid sequence of any one of SEQ ID NO: 16-22, or an amino acid sequence having at least 30%, preferably one of at least 35%, 40%, 45%, 50%, 55%, 60%, 65%, 70%, 75%, 80%, 85%, 90%, 91%, 92%, 93%, 94%, 95%, 96%, 97%, 98%, 99% or 100% amino acid sequence identity to the amino acid sequence of any one of SEQ ID NO: 16-22.

Fragments, variants, isoforms and homologues of an aldehyde dehydrogenase may optionally be characterised by the ability to catalyse conversion of butyrl-CoA to butyraldehyde.

As used herein, “aldehyde deformylating oxygenase” refers to an enzyme capable of catalysing the deformylation of aliphatic aldehydes for alkane biosynthesis. In particular, the present usage refers to enzymes capable of catalysing the deformylation of butyraldehyde to propane.

An exemplary aldehyde deformylating oxygenase is that from Procholorococcus marinus st. MIT9313 (ADO), as described in Menon N, et al Biotechnol Biofuels 2015; 8:61-12, which is herein incorporated by reference in its entirety. This enzyme catalyses the ferredoxin and oxygen-dependent decarbonylation of primarily long chain (C17-C19) fatty aldehydes into alkane hydrocarbons (C15-C17) and formate. Aldehyde deformylating oxygenase variants A134F and V41Y have enhanced propane production capabilities (Khara, B. et al. ChemBioChem 14 (10): 1204-1208).

The amino acid sequence of ADO is provided in SEQ ID NO:23. In some embodiments, an aldehyde deformylating oxygenase comprises, or consists of, the amino acid sequence of SEQ ID NO:23, or an amino acid sequence having at least 70%, preferably one of 80%, 85%, 90%, 91%, 92%, 93%, 94%, 95%, 96%, 97%, 98%, 99% or 100% amino acid sequence identity to the amino acid sequence of SEQ ID NO: 23. In some embodiments, an aldehyde deformylating oxygenase comprises an amino acid substitution at a position corresponding to position A134 of SEQ ID NO:23. In some embodiments, the amino acid substitution is A134F. In some embodiments, an aldehyde deformylating oxygenase comprises an amino acid substitution at a position corresponding to position V41 of SEQ ID NO:23. In some embodiments, the amino acid substitution is V41Y.

Fragments, variants, isoforms and homologues of an aldehyde deformylating oxygenase may optionally be characterised by the ability to catalyse conversion of butyraldehyde to butane.

In some embodiments, the aldehyde deformylating oxygenase may use an electron transfer partner protein, such as ferredoxin. In some embodiments, the aldehyde deformylating oxygenase may use a coenzyme, such as nicotinamide adenine dinucleotide (NAD⁺/NADH) or nicotinamide adenine dinucleotide phosphate (NADP⁺/NADPH). In some embodiments, ferredoxin interacts with NADP⁺/NADPH and the reducing equivalents of reduced ferredoxin are transferred to ADO. This electron transfer partner (acceptor/donor) can be a native ferredoxin encoded within the microbial host genome or it can be a recombinant ferredoxin. In particular, the present usage refers to the heterologous expression of the ferredoxin (fdx or PetF) from Synechocystis sp. PCC6803 (e.g. SEQ ID NO:38).

The metabolic pathway from CO₂ to bio-alkane may involve the activity of other enzymes. In some embodiments of the present disclosure, the Halomonas may express (either naturally or by genetic modification) one or more enzymes which can convert CO₂ to fatty acids and/or butyryl-CoA.

As used herein, an “acetyl-CoA acetyltransferase” refers to an enzyme capable of catalysis of the conversion of acetyl CoA to acetylacetyl-CoA. In some embodiments, the acetyl-CoA acetyltransferase comprises, or consists of, the amino acid sequence of SEQ ID NO:24, or an amino acid sequence having at least 70%, preferably one of 80%, 85%, 90%, 91%, 92%, 93%, 94%, 95%, 96%, 97%, 98%, 99% or 100% amino acid sequence identity to the amino acid sequence of SEQ ID NO:24.

As used herein, an “acetoacetyl COA synthase” refers to an enzyme capable of catalysis of the conversion of malonyl-CoA to acetylacetyl-CoA. In some embodiments, the acetoacetyl COA synthase comprises, or consists of, the amino acid sequence of SEQ ID NO:25, or an amino acid sequence having at least 70%, preferably one of 80%, 85%, 90%, 91%, 92%, 93%, 94%, 95%, 96%, 97%, 98%, 99% or 100% amino acid sequence identity to the amino acid sequence of SEQ ID NO:25.

As used herein, a “clostridial 3-hydroxybutyryl-CoA dehydrogenase” refers to an enzyme capable of catalysis of the conversion of acetylacetyl-CoA to 3-hydroxybutryryl-CoA. In some embodiments, the clostridial 3-hydroxybutyryl-CoA dehydrogenase comprises, or consists of, the amino acid sequence of SEQ ID NO:26, or an amino acid sequence having at least 70%, preferably one of 80%, 85%, 90%, 91%, 92%, 93%, 94%, 95%, 96%, 97%, 98%, 99% or 100% amino acid sequence identity to the amino acid sequence of SEQ ID NO:26.

As used herein, a “3-hydroxybutyryl-CoA dehydratase” refers to an enzyme capable of catalysis of the conversion of 3-hydroxybutryryl-CoA to crotonyl-CoA. In some embodiments, the clostridial 3-hydroxybutyryl-CoA dehydrogenase comprises, or consists of, the amino acid sequence of SEQ ID NO: 27, or an amino acid sequence having at least 70%, preferably one of 80%, 85%, 90%, 91%, 92%, 93%, 94%, 95%, 96%, 97%, 98%, 99% or 100% amino acid sequence identity to the amino acid sequence of SEQ ID NO:27.

As used herein, a “trans-enoyl-CoA reductase” refers to an enzyme capable of catalysis of the conversion of crotonyl-CoA to butyryl-CoA. In some embodiments, the clostridial 3-hydroxybutyryl-CoA dehydrogenase comprises, or consists of, the amino acid sequence of SEQ ID NO:28, or an amino acid sequence having at least 70%, preferably one of 80%, 85%, 90%, 91%, 92%, 93%, 94%, 95%, 96%, 97%, 98%, 99% or 100% amino acid sequence identity to the amino acid sequence of SEQ ID NO:28.

As used herein, an “acyl-CoA thioester hydrolase” refers to an enzyme capable of catalysis of the conversion of butyryl-coenzyme A to butyric acid. In some embodiments, the clostridial 3-hydroxybutyryl-CoA dehydrogenase comprises, or consists of, the amino acid sequence of SEQ ID NO:29, or an amino acid sequence having at least 70%, preferably one of 80%, 85%, 90%, 91%, 92%, 93%, 94%, 95%, 96%, 97%, 98%, 99% or 100% amino acid sequence identity to the amino acid sequence of SEQ ID NO:29.

As used herein “carboxylic acid reductase” or “CAR” refers to an enzyme converts butyrate to butyraldehyde. This enzyme contains the unusual cofactor phosphopantetheine. In some embodiments, carboxylic acid reductase is from Mycobacterium marinum. In some embodiments, carboxylic acid reductase has GenBank ID: ACC40567.1. In some embodiments, the carboxylic acid reductase comprises, or consists of, the amino acid sequence of SEQ ID NO:41, or an amino acid sequence having at least 70%, preferably one of 80%, 85%, 90%, 91%, 92%, 93%, 94%, 95%, 96%, 97%, 98%, 99% or 100% amino acid sequence identity to the amino acid sequence of SEQ ID NO:41.

As used herein “maturation factor phosphopantetheinyl transferase” or “Sfp” is an enzyme which produces an active phosphopantetheine cofactor. In some embodiments, the Sfp is from Bacillus subtilis. In some embodiments, the Sfp has GenBank ID: X65610.1. In some embodiments, the Sfp comprises, or consists of, the amino acid sequence encoded by SEQ ID NO:42, or an amino acid sequence having at least 70%, preferably one of 80%, 85%, 90%, 91%, 92%, 93%, 94%, 95%, 96%, 97%, 98%, 99% or 100% amino acid sequence identity to the amino acid sequence encoded by SEQ ID NO:42.

### Linalool Production

Linalool is a monoterpene which is mainly used as a fragrance material in 60-80% of perfumed hygiene products. It is widely used in cosmetic products like perfumes, lotions, soaps, and shampoos and also in non-cosmetic products like detergents and cleaning agents. Furthermore, during the manufacturing process of Vitamin E, linalool is a vital intermediate. As an important ingredient in a wide range of commercial products, the worldwide use of linalool exceeds 1000 metric tonnes per annum (Lapczynski, et al., 2008).

Both (R) and(S) isomers of linalool are found in nature with (R)-(−)-linalool being the most widely distributed in plant and flower extracts. The structure of (R) and(S) isomers of linalool are shown in FIG. 1. To our knowledge, for industrial use as a fragrance, the isomeric mixture is used. In this specification, reference to linalool includes (R)-linalool, (S)-linalool, and mixtures (e.g. racemic mixture) of (R)-linalool and(S)-linalool.

Linalool, as other monoterpenoids, is produced from isopentenyl pyrophosphate via the universal isoprenoid intermediate geranyl pyrophosphate, through a class of enzymes named monoterpene synthases.

Linalool synthase as used herein generally refers to an enzyme capable of catalysing the conversion of geranyl diphosphate (also known as geranyl pyrophosphate; GPP) and water to linalool and diphosphate. Thus, the two substrates of this enzyme are geranyl diphosphate and H₂O, whereas its two products are linalool and diphosphate. The reaction is initiated by the metal-dependent ionisation of geranyl diphosphate resulting in the geranyl cation, which undergoes an isomerisation (via linalyl diphosphate) to the linalyl cation, which upon water attack yields linalool.

More specifically, (S)-linalool synthases convert geranyl diphosphate and water to (3S)-linalool and diphosphate, and (R)-linalool synthases convert geranyl diphosphate and water to (3R)-linalool and diphosphate. (S)-linalool synthases have the Enzyme Commission number 4.2.3.25, and (R)-linalool synthases have the Enzyme Commission number 4.2.3.26.

In aspects and embodiments of the present invention a linalool synthase may be an S- or R-linalool synthase.

Linalool synthases have been identified and characterised in a wide range of species, including plants Arabidopsis thaliana (UniProtKB-Q84UV0), Oryza sativa (UniProtKB-Q6ZH94), Mentha aquatica (UniProtKB-Q8H2B4), and Ocimum basilicum (UniProtKB-Q5SBP3), and bacteria Streptomyces clavuligerus (UniProtKB-D5SL78).

Streptomyces clavuligerus linalool synthase (bLinS) is a 330 amino acid polypeptide consisting of the sequence of SEQ ID NO:30 (UniProtKB-D5SL78). Variants of the S. clavuligerus bLinS have been generated, as described in WO/2020/234307. Variants of the S. clavuligerus bLinS may comprise amino acid substitutions corresponding to one or more of the following positions numbered relative to the amino acid sequence of SEQ ID NO:1: L72 and V214. The variant of the S. clavuligerus bLinS may consist of the sequence of SEQ ID NO:31, SEQ ID NO:32, or SEQ ID NO:33.

In some embodiments of the present disclosure, the Halomonas is modified to express a heterologous linalool synthase from S. clavuligerus. In some embodiments, the linalool synthase comprises, or consists, of an amino acid sequence having at least 70%, 75%, 80%, 85%, 90%, 95%, 99%, or 100% sequence identity to SEQ ID NO: 30, SEQ ID NO: 31, SEQ ID NO: 32, and/or SEQ ID NO: 33.

In this specification “linalool synthase” refers to a linalool synthase from or derived from any species, in particular bacteria (e.g. S. clavuligerus) and plants (e.g. Mentha aquatica) and includes isoforms, fragments, variants or homologues of linalool synthase from any species. Homologues include orthologues.

Some organisms that can be utilised in the production of linalool and linalool derivatives do not naturally produce GPP, which is the substrate of the linalool synthases described in this invention. In such organisms, the host is engineered to produce GPP. In some embodiments, the host is transformed to express a GPP production platform.

Terpenoids are naturally synthesized from the universal C5 isoprenoid precursors isopentenyl diphosphate (IPP) and dimethylallyl diphosphate (DMAPP), the products of either the methylerythritol 4-phosphate (MEP) pathway, or the mevalonate-dependent (MVA) pathway

The GPP production platform comprises an exogenous, hybrid mevalonate (MVA) pathway and a GPP synthase. The hybrid MVA pathway (pMVA) comprises an acetyl-CoA acetyltransferase (atoB), HMG-CoA synthase (HMGS), HMG-COA reductase (HMGR), mevalonate kinase (MK), phosphomevalonate kinase (PMK), phosphomevalonate decarboxylase (PMD) and isopentenyl diphosphate isomerase (idi). The combination of the MVA pathway and GPP synthase enable the generation of GPP from acetyl-CoA, the latter of which is readily available in most hosts. In some embodiments, the GPP production platform is as described by Leferink et al. (2016).

In some embodiments of the present disclosure, the Halomonas may express (either naturally or by genetic modification) one or more enzymes which can convert acetyl CoA into IPP, for example by way of the mevalonate-dependent (MVA) pathway. In embodiments, the one or more enzymes may include, for example, acetoacetyl-CoA thiolase (AtoB, EC 2.3.1.9) (from E. coli for example), hydroxymethylglutaryl-CoA synthase (HMGS, EC 2.3.3.10) (from S. cerevisiae for example), hydroxymethylglutaryl-CoA reductase (HMGR, EC 1.1.1.34) (from S. cerevisiae for example), mevalonate kinase (MK, EC 2.7.1.36) (from S. cerevisiae for example), phosphomevalonate kinase (PMK, EC 2.7.4.2) (from S. cerevisiae for example), phosphomevalonate decarboxylase (PMD, EC 4.1.1.33) (from S. cerevisiae for example) and/or isopentenyldiphosphate isomerase (idi, EC 5.3.3.2) (from E. coli for example).

In some embodiments of the present disclosure, the Halomonas may express (either naturally or by genetic modification) one or more enzymes which can convert pyruvate into DMAPP, for example by way of the methylerythritol 4-phosphate (MEP) pathway. In some embodiments, the one or more enzymes may include, for example, 1-deoxyxylulose-5-phosphate synthase (DXS, EC 2.2.1.7), 1-deoxyxylulose-5-phosphate reductoisomerase (DXR, IspC, EC 1.1.1.267), 2-C-methyl-D-erythritol 4-phosphate cytidylyltransferase (YgbP, IspD, EC 2.7.7.60), 4-(cytidine 5′-diphospho)-2-C-methyl-D-erythritol kinase (YchB, IspE, EC 2.7.1.148), (E)-4-hydroxy-3-methylbut-2-enyl-diphosphate synthase (GcpE, IspG, EC 1.17.7.1) and/or 4-hydroxy-3-methylbut-2-en-1-yl diphosphate reductase (LytB, IspH, EC 1.17.7.4). In embodiments, such enzymes may be from E. coli, for example.

### Amine Production

Amines are compounds that contain a basic nitrogen atom with a lone pair. Amines are formally derivatives of ammonia (NH3), wherein one or more hydrogen atoms have been replaced by a substituent such as an alkyl or aryl group.

Amines can be classified according to the nature and number of substituents on nitrogen. Aliphatic amines contain only H and alkyl substituents. Aromatic amines have the nitrogen atom connected to an aromatic ring. It is a broad class of compounds that encompasses anilines, but also many more complex aromatic rings and many amine substituents beyond NH2. Such compounds occur widely.

In some embodiments, the amine is a primary amine. Primary amines arise when one of three hydrogen atoms in ammonia is replaced by an alkyl or aromatic group. Important primary alkyl amines include, methylamine, most amino acids, and the buffering agent tris, while primary aromatic amines include aniline and benzylamine.

In some embodiments, the amine is an aromatic amine. Aromatic amines are widely used as precursor to pesticides, pharmaceuticals, and dyes. Benzylamine, aniline, toluidines, and phenylenediamine are aromatic amines. In some embodiments, the amine is a primary aromatic amine. In some embodiments, the amine is benzylamine.

Benzylamine has the molecular formula C₇H₉N or C₆H₅CH₂NH₂, and is also known as phenylmethanamine, benzenemethanamine, and monobenzylamine. Benzylamine is a primary amine compound having benzyl as the N-substituent. It is a colourless to light yellow liquid with a strong odour of ammonia, and it floats and mixes with water. Benzylamine is a precursor to the synthesis of many commercial-important compounds including motion-sickness treatments, anticonvulsants and the high-energy propellant hexanitrohexaazaisowurtzitane (CL-20).

The Halomonas rowanensis, or the derivative of Halomonas rowanensis, may be used in methods of producing amines. The method may comprise the conversion of carboxylic acid and/or a conjugate base of a carboxylic acid (a carboxylate) to an aldehyde through CAR activity, and the conversion of an aldehyde to an amine through TA activity.

A carboxylic acid is an organic compound containing a carboxyl functional group. Aldehydes comprise a carbon connected by a double bond to oxygen (carbonyl), a single bond to hydrogen, and single bond to a third substituent.

In some embodiments, the derivative of Halomonas rowanensis expresses a carboxylic acid reductase (CAR) and a transaminase (TA). In some embodiments, the derivative of Halomonas rowanensis is genetically modified to express a heterologous CAR and/or a heterologous TA.

CAR enzymes comprise enzymatic activity which facilitates the conversion of carboxylic acids (e.g. benzoic acid) to aldehydes (e.g. benzaldehydes). Individual CAR enzymes, e.g. the Nocardia iowensis CAR, are capable of catalysing the reduction of a wide range of carboxylic acids.

CARs are relatively large, multidomain enzymes of around 130 kDa. They feature an N-terminal adenylation domain, a C-terminal thioester reductase domain that likely adopts a Rossmann fold, and a central phosphopantetheine binding domain (Marchler-Bauer et al., 2015. Nucleic Acids Res, 43, D222-D2226).

The CAR comprises carboxylic acid reductase activity. This activity can be assayed through any method known in the art, for example those described in Finnegan et al., 2017, ChemCatChem, 9 (6): https://doi.org/10.1002/cctc.201601249, Khusnutdinova et al., Biotechnol J. 2017 November; 12 (11): 10.1002/biot.201600751, and/or Marchler-Bauer et al., 2015. Nucleic Acids Res, 43, D222-D2226. To summarise, carboxylic acid reductase activity of purified CARs against different carboxylic acids can be determined spectrophotometrically using an NADPH oxidation-based assay by following the decrease in absorbance at 340 nm. In one example of this assay, a reaction mixture (0.2 ml) containing HEPES-K (100 mM, pH 7.5), 1 mM NADPH, 2.5 mM ATP, 10 mM MgCl2, 10 mM substrate (e.g. benzoic acid or decanoic acid), and 2.5-5.0 μg of purified CAR (10 min incubation at 30° C.) could be employed to assay CAR activity.

In some embodiments, the CAR comprises an amino acid sequence with at least 40% sequence identity to SEQ ID NO:43, 44, 45, 46, 47, or 48. In some embodiments, the CAR comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:43, 44, 45, 46, 47, or 48. In some embodiments, the CAR is a wild type or mutant CAR derived from Nocardia iowensis.

TAs, also known as aminotransferases, are a group of enzymes that mediate the transfer of an amine group between an amino acid and a keto acid. TA enzymes are capable of the enzymatic conversion of an aldehyde (e.g. benzaldehyde) to an amine (e.g. benzylamine). It has been shown that ω-transaminases (ω-TAs) can be efficient enzymes for the conversion of aldehyde to amine (Fuchs et al., 2012).

In some embodiments, the TA is an omega-TA (ω-TA). In some embodiments, the ω-TA comprises an amino acid sequence with at least 40% sequence identity to SEQ ID NO:49, 50, 51, 52, 53, or 54. In some embodiments, the ω-TA comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:49, 50, 51, 52, 53, or 54. In some embodiments, the ω-TA is a wild type or mutant ω-TA derived from Chromobacterium violaceum or from Vibrio fluvialis.

In some embodiments, the derivative of Halomonas rowanensis expresses a a phosphopantetheinyl transferase (PPTases) and/or an alanine dehydrogenase (AlaDH). In some embodiments, the derivative of Halomonas rowanensis that expresses the CAR and TA also expresses the PPTase. In some embodiments, the derivative of Halomonas rowanensis that expresses the CAR and TA also expresses the AlaDH. In some embodiments, the derivative of Halomonas rowanensis that expresses the CAR and TA also expresses the PPTase and the AlaDH.

Phosphopantetheinyl transferases (PPTases) are essential for cell viability across all three domains of life: bacteria, archaea and eukaryota. These enzymes are known to the skilled person, and reviewed, for example, in Beld et al. Nat Prod Rep. 2014 January; 31(1): 61-108.

Surfactin phosphopantetheinyl transferase (Sfp) are a family of PPTases. Sfp are utilised in this disclosure to enhance the loading of a phosphopantetheine group onto CAR enzymes. Any PPTase or Sfp may be suitable for this function. The PPTase/Sfp may be a wild type or mutant Bacillus subtilis PPTase/Sfp.

In some embodiments, the Sfp comprises an amino acid sequence with at least 40% sequence identity to the amino acid sequence encoded by SEQ ID NO:42. In some embodiments, the PPTase/Sfp comprises, or consists, of an amino acid sequence having at least 45%, 50%, 55%, 60%, 65%, 70%, 75%, 80%, 85%, 90%, 95%, 99%, or 100% sequence identity to the amino acid sequence encoded by SEQ ID NO: 42.

AlaDH (E.C.1.4.1.1) is an enzyme that catalyzes a reversible conversion of L-alanine to pyruvate. Inter-conversion of alanine and pyruvate by AlaDH is central to metabolism in microorganisms. These enzymes known to the skilled person, and reviewed, for example, by Dave and Kadeppagari, (Crit Rev Biotechnol. 2019 August; 39(5):648-664. doi: 10.1080/07388551.2019.1594153).

In some embodiments, the AlaDH comprises an amino acid sequence with at least 40% sequence identity to SEQ ID NO:55. In some embodiments, the AlaDH comprises, or consists, of an amino acid sequence having at least 45%, 50%, 55%, 60%, 65%, 70%, 75%, 80%, 85%, 90%, 95%, 99%, or 100% sequence identity to one or more of the amino acid sequences of SEQ ID NO:55. The AlaDH may be a wild type or mutant Bacillus subtilis AlaDH.

### Bioremediation

The process of bioremediation utilises biological organisms, such as microorganisms, to remove pollutants from the environment. As the microorganisms, such as Halomonas rowanensis, described herein are able to fix carbon dioxide and utilise other polluting compounds (e.g. sulfur containing compounds), they have strong potential for use in bioremediation.

The use of micro-organisms, such as Halomonas, in bioremediation required the microorganisms to be cultured in a polluted environment. In some embodiments, the environment is polluted water. In some embodiments, the Halomonas is cultured in an area with polluted air. In some embodiments, the Halomonas culture is provided with industrial waste.

In some embodiments, the Halomonas remediates carbon dioxide and/or sulfur compounds from the environment. In some embodiments, the Halomonas remediates carbon dioxide and/or sulfur compounds from the media. In some embodiments, the Halomonas remediates carbon dioxide and/or sulfur compounds from polluted water. In some embodiments, the Halomonas remediates carbon dioxide and/or sulfur compounds from the atmosphere.

In some embodiments, the Halomonas removes carbon dioxide and/or sulfur compounds from the environment. In some embodiments, the Halomonas removes carbon dioxide and/or sulfur compounds from the media. In some embodiments, the Halomonas removes carbon dioxide and/or sulfur compounds from polluted water. In some embodiments, the Halomonas removes carbon dioxide and/or sulfur compounds from the atmosphere.

Bioremediation may include the sequestering of environmental pollutants i.e. the accumulation of pollutants within a biological cell. In some embodiments, the Halomonas sequesters carbon dioxide and/or sulfur compounds from the environment. In some embodiments, the Halomonas sequesters carbon dioxide and/or sulfur compounds from the media. In some embodiments, the Halomonas sequesters carbon dioxide and/or sulfur compounds from polluted water. In some embodiments, the Halomonas sequesters carbon dioxide and/or sulfur compounds from the atmosphere.

The sulfur compound may be a thiosulfate, sulfuric acid, sulfur, a sulfate, and/or a sulfite.

### Sequence Identity

Pairwise and multiple sequence alignment for the purpose of determining percent identity between two or more amino acid or nucleic acid sequences can be achieved in various ways known to a person of skill in the art, for instance, using publicly available computer software such as ClustalOmega (Soding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6 (298)) and MAFFT (Katoh and Standley 2013, Molecular Biology and Evolution, 30 (4) 772-780 software. When using such software, the default parameters, e.g. for gap penalty and extension penalty, are preferably used.

Sequences

| SEQ | | |
|---|---|---|
| ID | | |
| NO: | DESCRIPTION | SEQUENCE |
| 1 | CvPAS/CvFAP | MASITSRASARASCSQANTRAGRVALSGGALLRPARPARSFVPARKQQQGAVRRGGALSARASA |
| | (Chlorella variabilis | VEDIRKVLSDSSSPVAGQKYDYILVGGGTAACVLANRLSADGSKRVLVLEAGPDNTSRDVKIPAAIT |
| | NC64A), Genbank ID: | RLFRSPLDWNLFSELQEQLAERQIYMARGRLLGGSSATNATLYHRGAAGDYDAWGVEGWSSED |
| | A0A248QE08 | VLSWFVQAETNADFGPGAYHGSGGPMRVENPRYTNKQLHTAFFKAAEEVGLTPNSDFNDWSHD |
| | | HAGYGTFQVMQDKGTRADMYRQYLKPVLGRRNLQVLTGAAVTKVNIDQAAGKAQALGVEFSTDG |
| | | PTGERLSAELAPGGEVIMCAGAVHTPFLLKHSGVGPSAELKEFGIPVVSNLAGVGQNLQDQPACL |
| | | TAAPVKEKYDGIAISDHIYNEKGQIRKRAIASYLLGGRGGLTSTGCDRGAFVRTAGQALPDLQVRFV |
| | | PGMALDPDGVSTYVRFAKFQSQGLKWPSGITMQLIACRPQSTGSVGLKSADPFAPPKLSPGYLTD |
| | | KDGADLATLRKGIHWARDVARSSALSEYLDGELFPGSGVVSDDQIDEYIRRSIHSSNAITGTCKMG |
| | | NAGDSSSVVDNQLRVHGVEGLRVVDASVVPKIPGGQTGAPVVMIAERAAALLTGKATIGASAAAP |
| | | ATVAA |
| 2 | CvPAS/CvFAP- | MASAVEDIRKVLSDSSSPVAGQKYDYILVGGGTAACVLANRLSADGSKRVLVLEAGPDNTSRDVKI |
| | with chloroplast | PAAITRLFRSPLDWNLFSELQEQLAERQIYMARGRLLGGSSATNATLYHRGAAGDYDAWGVEGW |
| | targeting sequence | SSEDVLSWFVQAETNADFGPGAYHGSGGPMRVENPRYTNKQLHTAFFKAAEEVGLTPNSDFND |
| | replaced with a | WSHDHAGYGTFQVMQDKGTRADMYRQYLKPVLGRRNLQVLTGAAVTKVNIDQAAGKAQALGVE |
| | methionine residue | FSTDGPTGERLSAELAPGGEVIMCAGAVHTPFLLKHSGVGPSAELKEFGIPVVSNLAGVGQNLQD |
| | | QPACLTAAPVKEKYDGIAISDHIYNEKGQIRKRAIASYLLGGRGGLTSTGCDRGAFVRTAGQALPDL |
| | | QVRFVPGMALDPDGVSTYVRFAKFQSQGLKWPSGITMQLIACRPQSTGSVGLKSADPFAPPKLSP |
| | | GYLTDKDGADLATLRKGIHWARDVARSSALSEYLDGELFPGSGVVSDDQIDEYIRRSIHSSNAITGT |
| | | CKMGNAGDSSSVVDNQLRVHGVEGLRVVDASVVPKIPGGQTGAPVVMIAERAAALLTGKATIGAS |
| | | AAAPATVAA |
| 3 | AaPAS (Aureococcus | MGRTLVLKVATTSYDYIIAGGGTAGCVLANRLSEDPSKKVLVLEAGDRGPNSPLVKIPVAILKLFKS |
| | anophagefferens) | AYDWNFATRPSEAVADRSLYVCRGKGLGGSSLTNVMLYNRGSANDYDAWAAACGDDSWGAEE |
| | UniProt ID: F0YAB1 | MLGYFKKAEDCLVPAHRANHYHGVGGPYASSHVPYTNEMSTAFVEAAVEDGGVRNGDFNDWST |
| | | SQVGFGRFAVSQRKGARVDAATAYLPRKVRRRANLDVVRGAALSGVTWNANKATGVEFAFGGV |
| | | SGIACGGEVILSGGAVHSPQMLMLSGVGAKAQLEEFGIPVVADRPGVGKNLQDHPACLVSWRGS |
| | | AKAQGKSHSTQLRIPGTTKTSPKALLQWLFLGRGPLASPGCDHGGFAKVGAGDGDCDVQFRFLA |
| | | TKSITPDGMSTISDSYEAAVDHPDGLTIQTIVARPKSRAGEVKLASRDPAAKPVIENAYLSDEADVM |
| | | TMVKALQKARSIASRAPLSAYAGHEEFPGEDVADERQLAAYVRNTAHTANAVVGTCKMGESSDAL |
| | | AVVDNHLKVIGVSNLRVVDASVMPTLPGGQTAASTVALAEKAADLIKGG |
| 4 | CcPAS (Chondrus | MASPCPAFATPIAVPRSTLTSLISSSSSCTPRPVRTPAPPTHRRLIHMAAPAGTVASTFRRTVPSSE |
| | crispus) UniProt ID: | AATTYDYIIVGGGAAGCVLANRLTEDPSTRVLLLEAGKPDDSFYLHVPLGFPYLLGSPNDWAFVTE |
| | R7Q9C0 | PEPNLANRRLYFPRGKVLGGSHAISVMLYHRGHPADYTAWAESAPGWAPQDVLPYFLKSESQQS |
| | | AVPNQDAHGYEGPLAVSDLARLNPMSKAFIKAAHNAAGLNHNPDFNDWATGQDGVGPFQVTQR |
| | | DGSRESPATSYLRAAKGRRNLTVMTGAVVERILFENPAGSSTPVATAVSFIDSKGTRVRMSASREI |
| | | LLCGGVYATPQLLMLSGVGPAEHLRSHGIEIVADVPAVGQNLQDHAAAMVSFESQNPEKDKANSS |
| | | VYYTERTGKNIGTLLNYVFRGKGPLTSPMCEAGGFAKTDPSMDACDLQLRFIPFVSEPDPYHSLAD |
| | | FATAGSYLQNRANRPTGFTIQSVAARPKSRGHVQLRSTDVRDSMSIHGNWISNDADLKTLVHGVK |
| | | LCRTIGNDDSMKEFRGRELYPGGEKVSDADIEAYIRDTCHTANAMVGTCRMGIGEQAAVDPALQV |
| | | KGVARLRVVDSSVMPTLPGGQSGAPTMMIAEKGADLIRAAARQADAATVGAAA |
| 5 | ChPAS | MMRRLVYICAVATVTAAISSRSVPTSARRLIALRGGVAAAEQLAEEPWDYIIVGGGAAGCVMAERL |
| | (Chrysochromulina | SAAEARVLVLEAGTDASRDLRIRVPAGLIKVFKSERDWDFTTEAGQGTSGRGIYLCRGKALGGSS |
| | sp. CCMP291) | CTNVMLYNRGSPADYNSWVAAGAEGWGPDSVLHYYRKSENYVGGASQYHGVDGPLSVSDVPY |
| | UniProt ID: | ENELSTAFLRAAGELGYRRVHDFNDWSAPQEGFGRYKVTQRNGERCSAANAYLEGTEGRSNLC |
| | A0A0M0JFC3 | VRTGVHATRVTLEGSGDDLCAAGVEYIGADGKPSRAQLAQGGEVLLSAGAVQSPQLLMLSGIGPR |
| | | AHLEEVGIEVRKELDNVGVGLADHPAVVVSCGSKKKVSVTDEIRLWGGSKTNPMALLRWLLWRR |
| | | GPLTSVACEFGGFFKTKPDLKQADVQVRFVAARAMSPDGITTLQQLGAGAKFLSGYTTQIIACRPQ |
| | | STGLVRLRSSDPLAQPMLQDVHLSDDADVATLREGIKLGRQLLAAKSFDQYRDEEVYPGVAVQSD |
| | | EDIDAYVRKTTHSANALVGSCRMGRVDDQAAVLDPEMRVRGVGSLRVVDASAMPHIIGGQTCGP |
| | | TIMMAEKAADLVLRQRAEINAYMQQAQAYLAASAGAATPALSPAQAA |
| 6 | CmPAS | MRSRYCFLLSSTPCKYAGQRSPFPASALAGVCAGGRLRNVTRNLRPGLRTLRASAETEHSQGTR |
| | (Cyanidioschyzon | QAQYDFIIVGAGAAGCVLANRLSTAQFSNGDRRYPRVLLLEAGDALAEAPYFEHIPLGFPQLIGSRL |
| | merolae) UniProt ID: | DYGFFSRENPTHLGGRGAVYLPRGRGEGGSHAISVMLVHRGSRHDYETWVKDYEALGWGPDDV |
| | M1VK13 | LPYFKRLESNERTAQRGADGEAATALHGSDGPLRVSDQRSPNPLSLAFIEACLERGIRRNKDFND |
| | | WDHGQEGAGLFQVTQRDGRRESPATAYLQPVRSRRNLHIETNALAEHLVWSKDGRRVEGIRFID |
| | | RHGRRRAALAHCEVILAAGAINTPQLLMLSGLGPGAHLQDFGIPVVRDLPGVGQNLQDHAAVMLS |
| | | YYAPDPYGKDRDKKRIFYTERLGKDPLVLAEYFLLGRGPLTSPVCEAGAFVHTQAVIGEPSCDLQL |
| | | RFVPFFSDADPYKSLGEYRSGGHVLTNTSIRPAGFGLQAVAIRPRSRGRIELATIDPRARPIIHTGW |
| | | LEDKRDLQTLLSGLKLGREILSGDSMRPYRGREAFPETLEDDLVTYIRRTCHTANAIVGTARMGTG |
| | | RDAVVDPELRVHGVERLRVIDASVMPKIIGGQTGVPTMMIAERGADLVKKTWKLV |
| 7 | CrPAS | MMLGPKTVTRGATKGAAPRSMAARRVGGARRLSVRAAAGPAGSEKFDYVLVGGGTASCVLANK |
| | (Chlamydomonas | LSADGNKKVLVLEAGPTGDAMEVAVPAGITRLFAHPVMDWGMSSLTQKQLVAREIYLARGRMLG |
| | reinhardtii) UniProt | GSSGSNATLYHRGSAADYDAWGLEGWSSKDVLDWFVKAECYADGPKPYHGTGGSMNTEQPRY |
| | ID: A8JHB7 | ENVLHDEFFKAAAATGLPANPDFNDWSHPQDGFGEFQVSQKKGQRADTYRTYLKPAMARGNLK |
| | | VVIGARATKVNIEKGSSGARTTGVEYAMQQFGDRFTAELAPGGEVLMCSGAVHTPHLLMLSGVGP |
| | | AATLKEHGIDVVSDLSGVGQNLQDHPAAVLAARAKPEFEKLSVTSEVYDDKCNIKLGAVAQYLFQR |
| | | RGPLATTGCDHGAFVRTSSSLSQPDLQMRFVPGCALDPDGVKSYIVFGELKKQGRAWPGGITLQL |
| | | LAIRAKSKGSIGLKAADPFINPAININYFSDPADLATLVNAVKMARKIAAQEPLKKYLQEETFPGERA |
| | | SSDKDLEEYIRRTVHSGNALVGTAAMGASPAAGAVVSSADLKVFGVEGLRVVDASVLPRIPGGQT |
| | | GAATVMVAERAAALLRGQATIAPSRQPVAV |
| 8 | CsPAS (Coccomyxa | MMASQSVFLGTRPATRSPLPIGRAGHGSAGRRALRVRAIIKSDNPAADKYDFILVGGGTAGCVLAN |
| | subellipsoidea) | RLTADGSKKVLLLEAGGANKAREVRTPAGLPRLFKSALDWNLYSSLQQAASDRSIYLARGKLLGG |
| | UniProt ID: 10YJ13 | SSATNATLYHRGTAADYDAWGVPGWTSQDALRWFIQAENNCRGIEDGVHGTGGLMRVENPRYN |
| | | NPLHEVFFQAAKQAGLPENDNFNNWGRSQAGYGEFQVTHSKGERADCFRMYLEPVMGRSNLTV |
| | | LTGAKTLKIETEKSGGATVSRGVTFQVNGQDGSKHSAELAAGGEVVLCAGSIHSPQILQLSGIGPQ |
| | | AELRSKDIPVVADLPGVGQNMQDHPACLSAFYLKESAGPISVTDELLHTNGRIRARAILKYLLFKKG |
| | | PLATTGCDHGAFVKTAGQSEPDLQIRFVPGLALDPDGIGSYTAFGKMKDQKWPSGITFQLLGVRP |
| | | KSRGSVGLRSDDPWDAPKLDIGFLTDKEGADLATLRSGIKLSREIAAEPAFGAYVGNELHPGAAAS |
| | | SDSAIDSFIRDTVHSGNANVGTCSMGVNGNAVVDPSLRVFGIRGLRVADASVIPVIPGGQTGAATV |
| | | MVAERAAEILLGSNQKQPAAAVPAAQPALA |
| 9 | GpPAS (Gonium | MMLGRKPVAPAKGASAARTVRPVRLAGGRRQLVVSAAAAPVDPAEKYDYILVGGGTAGCVLANK |
| | pectorale) | LSADGNKKVLVLEAGPSGDSLEVAVPAGIARLFAHPVMDWGMSSLTQKQLVAREIYLARGRLLGG |
| | | SSGTNATLYHRGTSSDYDSWGLEGWTSKDVLDWFVKAECYGDGPKPYHGNSGSMNVEQPRYQ |
| | | NPLHEEFFRAAAAAGIPANPDFNDWSRPQDGYGEFQVAQNKGQRADTYRTYLKPALSRGNLKVV |
| | | TGARTTKVHIEKGSSGPRARGVEFATQQFGDRYSAQLAPGGEVLMCTGAVHTPHLLMLSGVGPA |
| | | AALREHGVDVVADLAGVGANLQDHPAAVVAVRAKPEFEKLSVTSEIYDEKCNIKLGAVAQYLFNRR |
| | | GPLATTGCDHGAFVRTSGSHSQPDLQMRFVPGCALDPDGVKSYIVFGELKKQGRAWPGGITLQL |
| | | LAIRAKSKGSIGLKAADPFINPAININYFSDPADLATLKQGVRMARDIARQEPLRKYLQEETFPGERA |
| | | SSDSDIEEYVRRTVHSGNALVGTCAMGTSPAKGAVVSSSDLKVFGVEGLRVVDASVLPQIPGGQT |
| | | GAATVMVAERAAALLKGQTTMAPSRQPVAA |
| 10 | PtPAS | MYDYIICGGGLAGCVLAERLSQDESKRVLVLEAGGSDYKSLFIRIPAGVLRLFRSKYDWQHETGGE |
| | (Phaeodactylum | KGCNGRNVFLQRGKILGGSSCTNVCLHHRGSAEDYNSWNIPGWTATDVLPFFKQSQKDETGRDA |
| | tricornutum) | TFHGADGEWVMDEVRYQNPLSKLFLEVGEAAGLGTNDDFNNWSHPQDGVGRFQVSEVNGERC |
| | | SGATAFLSKAAKRSNVIVRTGTMVRRIDFDETKTAKGITYDLMGDDTCTVPCLKEGGEVLVTGGAI |
| | | ASPQLLMCSGIGPGKHLRSLGIPVVHDNSAVGENLQDHPAAVVSFKTPQKGVSVTSKLRLFGKTN |
| | | PIPVFQWLFFKSGLLTSTGCDHGAFVRTSDSLEQPDLQIRFLAARALGPDGMTTYTKFRTMKTVED |
| | | GYSFQSVACRAKSKGRIRLSSSNSHVKPMIDGGYLSNQDDLATLRAGIKLGRMLGNRPEWGEYLG |
| | | QEVYPGPDVQTDEEIDEYIRNSLHTANALTGTCKMGTGRGAVVGPDLRVIGVNGVRVADSSVFPCI |
| | | PGGQTATPTVMIADRAAVFVR |
| 11 | EhPAS (Emiliania | MVALFALQLALSPPQARLGSGSARAALRLRGGSGVTGGSLGRGGGSPAIDGEFDYIIVGGGAAGC |
| | huxleyi) UniProt ID: | VLANRLSADPAHRVLLIEAGGDASRDKRAQVPWAFTKLLRSEYDWDFHVEAEAAVNQQEVYLCR |
| | R1FBM9 | GKALGGSSVTNVMLYHRGSPADYDAWEEAGARGWGAKDVLPYYLRVEDYGDGASQYHAVGGH |
| | | VSVQEVPYQNQLSATFLRAMGQLGFRPNGDFNDWSSPQEGYGRYKVTQRAGRRCTAADGYLAA |
| | | ARERANLVVVTGAQATRLALDSAYDGAGRLQVSGVEFARGDEREPCSVRLARGGEAVLCAGAVQ |
| | | TPHLLLLSGIGPAEHLREVGVPVRADLPGVGSGLQDHPAVVVSYESKKAVAATDDALLKGYASLVN |
| | | PLAMLRWLLFGRGPLACAACDHGGFVRSSPDLDQPDVQIRFVPARASSASGMNTLIELGRRARFL |
| | | PGFSTQVVACRPRSEGRVRLRSADPFAKPIIEGIHLGAAEDVASLRHGIRLGRQVCAAAAFDEYRG |
| | | EEVFPGAAVQSDEQIDEYIRSSVHSANALTSSCRMGDPSDPAAVLDSHLRVRGVGGLRVADASA |
| | | MPRIIGGQTQAPTYMLAERAADILLHARLQAHEPATESVSQRLEVAAAAL |
| 12 | EhPAS2 (Emiliania | MSARWLLLLATHCSAALRNPFRAAPTHFDYIIVGGGTAGCVLADRLSAASKQVLVLEPGPSPAAEL |
| | huxleyi) UniProt ID: | KIAAPVALTKLFGSEYDWGFRSAPAPGTAGREVHLCRGKCLGGSSATNALLYLRGTAADFDGWG |
| | R1E6L1 | LDGWGSEAMLASFLAVEAQRDAAFRTDALHHGSGGAVPAETPRYANPLSERFLEAAAQAGHPSN |
| | | ADFNDWSRPQAGVGRFQLTTRRGRRAHSAATHLRRAARRPNLHVRCGCAATRLLLEAEGGGGG |
| | | GGGGGKTRPWTGPAVTGQAGRRAVGVEYIDAAGVQRTASVSGGGGGGGGEVLLCAGAVSSPH |
| | | LLLLSGIGSPDELAAHGIGAEVCLPGVGRNLIDQPAVVTGYTVTSPLSITDEMFWRRSGALSPRRV |
| | | GEWLLRGSGPLASSGCDFGGFFSSRPGLAQPDLQLRFVPGLGTSPDGVSSYRDIGRAGKTPSGL |
| | | TLQSIAVRPTARGSVSLSSADPSAPPRIETGYGTSEADLATLRQGLRLSRELVAQPAFDGVRGEEA |
| | | WPRAACRLRRPGDDAALDEYIRSTAHSANALGGSCRMGRATSPARLVEGSDPLAVVDPALRVRG |
| | | ASGLRVVDASVLPTLPGGQLGATTFALAERAARIILGERAAGEAEAPAERRQEHAHALGAA |
| 13 | NgPAS | MSSNGYLRAYHLLIALLISANAFLITPPRLSKTTIGLQSFVTANYGVRRAISLRGGLQSVSMKAPAAV |
| | (Nannochloropsis | ASSTYDYIIVGGGIGGCVLANRLTESGRFKVLLLEAGKSAERNPYVNIPAGVVRLFKSALDWQFESA |
| | gaditana) UniProt ID: | PERHLDGKEVYLVRGKAMGGSSAVNVMLVHRGSASDYAKWEAEGAQGWGPEEALRYFKKMED |
| | W7TN63 | NLVGGEGRWHGQGGMYPVDDVKYQNPLSKRFLQACEEYGWRANPDFNDWSHPQDGYGSFKV |
| | | AQKHGKRVTAASGYLNKAVRRRPNLDILSEALVTRVLLEGEGDVKAVGVEFTGKDGKTHQVRTTG |
| | | KAGEVLLAGGAVNSPQLLMLSGIGPEADLQAVGIATKVNRPGVGENLQDHPAVTIAHNITRPISLCD |
| | | DLFLFHTPVPKPHQVLRWTLTGSGPLTTPGCDHGAFLKTREDLQEPNVQFRFIAGRGSDPDGVRS |
| | | YIMGGSARPLSGLTLQVVNIRPKSKGKLTLASKDPLKKPRIEVRYLSAAEDLQALRTGMRIGRDLIK |
| | | QRAFADILDEEVFPGPAAQTDEELDAYIRDSLHTANALVGTCKMGSVEDRNAVVDPECRVIGVGG |
| | | LRVVDASVMPVIPGGQTGSGTTMLAEKAADLVRAHAGDLVEMGVQDEERKGGWFNGLLGRKQK |
| | | VATEKERGERGKSERFVSEVIRHMGRVFVQVSRARRAQTCMRVGKGLDRERQLECAMRKELTIA |
| | | LFYAMLFTMRHSGFLSTTGRASYKDLGYLTGSCRAHPCTSPSSLCLFPEKPFMKLSPALAVVGFC |
| | | FNSINVQGFLLSNLAGRSLKHPVPQKGLYSRIEYDAREPRLDEFGLPLDPADLMEKPRVPLKDRVY |
| | | HIIDMTNDWVDAVSRGRREEETRRIIQRRRAAAKAMAIKDKVLISLDYVFHPVKAWRTFVADPLEAR |
| | | HQRQLRQQAEKRARLERYLQRYNTVKNRFHDTLDLLESTTRTSVKVAKSVSSAVVGAPGTVTRTV |
| | | KEVKSQAQGTAEAVAKVSSSVSSVVSKITSVIRKEDGALAGAKGKKDPRSEDEGKADPVKVREIW |
| | | ETKEQTAIRTIWEADELVTPVTPPATAMASTVSVSEPQDENEASISQGAAPSPSTSSPSSPEPVTR |
| | | LSFRARVEADEKERFGSRRLKISGNVPPTASPTRGASSLPLDTLSSSATQTFERSKVGPPIRTSKA |
| | | RCIGKCVHNGWKGICEEWFVHISFPTYAVSIVRPPMHVHNFKVICCVLAVRHARRKKEMSTALSTH |
| | | LIYLLLKTVKMLQDLPQLRRKGKTN |
| 14 | VcPAS (Volvox carteri | MLLGQRPFGAPAKGAMPCWKAARHGGVAGVARRPVAVKAAASVGSEKFDYILVGGGTAGCVLA |
| | f. nagariensis) | NKLSANGSKKVLVLEAGPTGDAMEVAVPAGIARLFAHPVFDWGMSSLTQQQLVAREIYLARGRLL |
| | UniProt ID: D8TNQ4 | GGSSGTNATLYHRGTPADYDSWGLEGWTSKDLLDWFVKAECYGDGPRAFHGQSGSMNVEQPR |
| | | YQNVLHDEFFRAAAAAGLPANEDFNDWSRPQEGYGEFQVAQKNGERADTYRTYLKPAMGRDNL |
| | | KVMTGARTTKVHIEKSSTGPRARGVEYATQQFGERYTAELTPGGEVLMCTGAVHTPHLLMLSGIG |
| | | PAPTLLEHGLDVISSLPGVGANLQDHPAAVLAVRAKPEFEGLSVTSEIYDSKCNIRLGAVMKYLFGR |
| | | RGPLATTGCDHGAFVRTSASHSQPDLQMRFVPGCALDPDGVKSYIVFGELKKQGRAWPGGITLQ |
| | | LLGIRAKSRGSIGLKAADPFINPAININYFSDPEDLATLKNGVRIAREIVAQEPLRKYLLEETFPGERA |
| | | NTDKDIEEYVRRTVHSGNALVGTCAMGTTPASGAVVSSADLKVFGVDGLRVVDASVLPRIPGGQT |
| | | GAATVMVAERAAAMLLGQATITSRREPAAV |
| 15 | Fatty acid | G-X1-L-X2-X3-X4-X5-C-[D/E]-X6-G-[A/G]-F-X7-[K/R] |
| | photodecarboxylase | X being any amino acid |
| | consensus sequence | |
| 16 | BALDH (Clostridium | MNKDTLIPTTKDLKVKTNGENINLKNYKDNSSCFGVFENVENAISSAVHAQKILSLHYTKEQREKIIT |
| | beijerinckii) UniProt | EIRKAALQNKEVLATMILEETHMGRYEDKILKHELVAKYTPGTEDLTTTAWSGDNGLTVVEMSPYG |
| | ID: Q716S8 | VIGAITPSTNPTETVICNSIGMIAAGNAVVFNGHPCAKKCVAFAVEMINKAIISCGGPENLVTTIKNPT |
| | | MESLDAIIKHPSIKLLCGTGGPGMVKTLLNSGKKAIGAGAGNPPVIVDDTADIEKAGRSIIEGCSFDN |
| | | NLPCIAEKEVFVFENVADDLISNMLKNNAVIINEDQVSKLIDLVLQKNNETQEYFINKKWVGKDAKLF |
| | | LDEIDVESPSNVKCIICEVNANHPFVMTELMMPILPIVRVKDIDEAIKYAKIAEQNRKHSAYIYSKNID |
| | | NLNRFEREIDTTIFVKNAKSFAGVGYEAEGFTTFTIAGSTGEGITSARNFTRQRRCVLAGLEHHHHHH |
| 17 | PduP (Salmonella | MNTSELETLIRTILSEQLTTPAQTPVQPQGKGIFQSVSEAIDAAHQAFLRYQQCPLKTRSAIISAMRQ |
| | typhimurium) UniProt | ELTPLLAPLAEESANETGMGNKEDKFLKNKAALDNTPGVEDLTTTALTGDGGMVLFEYSPFGVIGS |
| | ID: H9L416 | VAPSTNPTETIINNSISMLAAGNSIYFSPHPGAKKVSLKLISLIEEIAFRCCGIRNLVVTVAEPTFEAT |
| | | QQMMAHPRIAVLAITGGPGIVAMGMKSGKKVIGAGAGNPPCIVDETADLVKAAEDIINGASFDYNLPC |
| | | IAEKSLIVVESVAERLVQQMQTFGALLLSPADTDKLRAVCLPEGQANKKLVGKSPSAMLEAAGIAVP |
| | | AKAPRLLIALVNADDPWVTSEQLMPMLPVVKVSDFDSALALALKVEEGLHHTAIMHSQNVSRLNLA |
| | | ARTLQTSIFVKNGPSYAGIGVGGEGFTTFTIATPTGEGTTSARTFARSRRCVLTNGFSIR |
| 18 | Acetaldehyde | MLSRQNARELVRNAKQAQVIMATFSQQKIDAIVKNVAEEAARHAETLAKMAAEETGFGNWQDKVL |
| | dehydrogenase | KNRFASLHVYDAIKEMKTVGIIHDDQAKKVMDVGVPLGVICALVPSTNPTSTIFYKTLIALKAGNAIIF |
| | (Acetylating) | SPHPGARQCSWKAIEIVKRAAEAAGAPAGIVDGVTQLTLEATSELMHSKDVSLILATGGEGMVRAA |
| | (Aeromonas | YASGTPTISGGPGNGPAFIERSADIHQAVKDIITSKTFDNGVICASEQSIIVERCIYDEVHRELAAQGA |
| | hydrophila subsp. | YFMNEDEAARMAALLLRPNGTINPKVVGKTALHLSQLAGFSVPPSTRVLVAEQTTVSHSNPYSRE |
| | hydrophila) UniProt | KLCPVLGLYVEEEWRAACHRVVELLTNEGLGHTLVIHTRNQDVIRQFSLEKPVNRILINTPAALGGI |
| | ID: A0KHX2 | GATTNLTPALTLGCGAVGGGSSSDNVGPMNLLNIRKVGYGVRTIEELRAPIQPVAVQPASAAPTAP |
| | | QPCSILDDARFSAPAPACHSADDRFAGASAEVGGEISEQNVERVIRQVLERLGK |
| 19 | Aldehyde | MNTAELETLIRTILSEKLAPTPPAPQQEQGIFCDVGSAIDAAHQAFLRYQQCPLKTRSAIISALRETL |
| | dehydrogenase (NAD) | APELATLAEESATETGMGNKEDKYLKNKAALENTPGIEDLTTSALTGDGGMVLFEYSPFGVIGAVA |
| | family protein | PSTNPTETIINNSISMLAAGNSVYFSPHPGAKKVSLKLIARIEEIAYRCSGIRNLVVTVAEPTFEATQQ |
| | (Klebsiella | MMSHPLIAVLAITGGPGIVAMGMKSGKKVIGAGAGNPPCIVDETADLVKAAEDIISGAAFDYNLPCIA |
| | pneumoniae subsp. | EKSLIVVASVADRLIQQMQDFDALLLSRQEADTLRAVCLPDGAANKKLVGKSPAALLAAAGLAVPP |
| | rhinoscleromatis | RPPRLLIAEVEANDPWVTCEQLMPVLPIVRVADFDSALALALRVEEGLHHTAIMHSQNVSRLNLAA |
| | ATCC 13884) UniProt | RTLQTSIFVKNGPSYAGIGVGGEGFTTFTIATPTGEGTTSARTFARLRRCVLTNGFSIR |
| | ID: C8SXL7 | |
| 20 | PduP_L.bre aldehyde | MNTENIEQAIRKILSEELSNPQSSTATNTTVPGKNGIFKTVNEAIAATKAAQENYADQPISVRNKVID |
| | dehydrogenase EutE | AIREGFRPYIEDMAKRIHDETGMGTVSAKIAKLNNALYNTPGPEILQPEAETGDGGLVMYEYAPFG |
| | (Lactobacillus brevis) | VIGAVGPSTNPSETVIANAIMMLAGGNTLFFGAHPGAKNITRWTIEKLNELVADATGLHNLVVSLET |
| | UniProt ID: Q03Q42 | PSIESVQEVMQHPDVAMLSITGGPAVVHQALISGKKAVGAGAGNPPAMVDATANIALAAHNIVDSA |
| | | AFDNNILCTAEKEVVVEAAVKDELIMRMQQEGAFLVTDSADIEKLAQMTIGPKGAPDRKFVGKDAT |
| | | YILDQAGISYTGTPTLIILEAAKDHPLVTTEMLMPILPVVCCPDFDSVLATATEVEGGLHHTASIHSEN |
| | | LPHINKAAHRLNTSIFVVNGPTYCGTGVATNGAHSGASALTIATPTGEGTATSKTYTRRRRLNSPE |
| | | GFSLRTWEA |
| 21 | EutE ethanolamine | MESLELEKLVKKVLLEKLAEQKGIPVKTMTKGAKSGVFDTVDEAVQAAVIAQNSYKEKSLEERRNV |
| | utilization protein | VKAIREALYPEIESIAARAVAETGMGNVADKILKNTLAIEKTPGVEDLYTEVATGDNGMTLYELSPYG |
| | (Listeria | VIGAVAPSTNPTETLICNTIGMLAAGNAVFYSPHPGAKNISLWLIEKLNTIVRESCGVDNLVVTVEKP |
| | monocytogenes EGD-e) | SIQAAQEMMNHPKVPLLVITGGPGVVLQAMQSGKKVIGAGAGNPPSIVDETANIEKAAADIVDGAS |
| | UniProt ID: | FDHNILCIAEKSVVAVDSIADFLMFQMEKNGALHVTNPSDIQKLEKVAVTDKGVTNKKLVGKSASEI |
| | Q8Y7V4 | LKEAGIACDFSPRLIIVETEKTHPFATVELLMPIVPVVRVPNFEEALEVAIELEQGLHHTATMHSQNIS |
| | | RLNKAARDMQTSIFVKNGPSFAGLGFRGEGSTTFTIATPTGEGTTTARHFARRRRCVLTDGFSIR |
| 22 | Succinate- | MEIKEMVSLARKAQKEYQATHNQEAVDNICRAAAKVIYENAAILAREAVDETGMGVYEHKVAKNQ |
| | semialdehyde | GKSKGVWYNLHNKKSVGILNIDERTGMIEIAKPIGVVGAVTPTTNPIVTPMSNIIFALKTCNAIIIAPH |
| | dehydrogenase | PRSKKCSAHAVRLIKEAIAPFNVPEGMVQIIEEPSIEKTQELMGAVDVVVATGGMGMVKSAYSSGKP |
| | (Porphyromonas | SFGVGAGNVQVIVDSNIDFEAAAEKIITGRAFDNGIICSGEQSIIYNEADKEAVFTAFRNHGAYFCDE |
| | gingivalis) Genbank | AEGDRARAAIFENGAIAKDVVGQSVAFIAKKANINIPEGTRILVVEARGVGAEDVICKEKMCPVMCA |
| | ID: WP_012457729 | LSYKHFEEGVEIARTNLANEGNGHTCAIHSNNQAHIILAGSELTVSRIVVNAPSATTAGGHIQNGLA |
| | | VTNTLGCGSWGNNSISENFTYKHLLNISRIAPLNSSIHIPDDKEIWEL |
| 23 | ADO | MPTLEMPVAAVLDSTVGSSEALPDFTSDRYKDAYSRINAIVIEGEQEAHDNYIAIGTLLPDHVEELK |
| | (Prochlorococcus | RLAKMEMRHKKGFTACGKNLGVEADMDFAREFFAPLRDNFQTALGQGKTPTCLLIQALLIEAFAIS |
| | marinus MIT9313), | AYHTYIPVSDPFARKITEGVVKDEYTHLNYGEAWLKANLESCREELLEANRENLPLIRRMLDQVAG |
| | UniProt ID: Q7V6D4 | DAAVLQMDKEDLIEDFLIAYQESLTEIGFNTREITRMAAAALVS |
| 24 | Acetyl-CoA | MKNCVIVSAVRTAIGSFNGSLASTSAIDLGATVIKAAIERAKIDSQHVDEVIMGNVLQAGLGQNPAR |
| | acetyltransferase | QALLKSGLAETVCGFTVNKVCGSGLKSVALAAQAIQAGQAQSIVAGGMENMSLAPYLLDAKARSG |
| | AtoB (E.coli), | YRLGDGQVYDVILRDGLMCATHGYHMGITAENVAKEYGITREMQDELALHSQRKAAAAIESGAFT |
| | Genbank ID: P76461 | AEIVPVNVVTRKKTFVFSQDEFPKANSTAEALGALRPAFDKAGTVTAGNASGINDGAAALVIMEES |
| | | AALAAGLTPLARIKSYASGGVPPALMGMGPVPATQKALQLAGLQLADIDLIEANEAFAAQFLAVGK |
| | | NLGFDSEKVNVNGGAIALGHPIGASGARILVTLLHAMQARDKTLGLATLCIGGGQGIAMVIERLN |
| 25 | Acetoacetyl COA | MTDVRFRIIGTGAYVPERIVSNDEVGAPAGVDDDWITRKTGIRQRRWAADDQATSDLATAAGRAA |
| | synthase NphT7 | LKAAGITPEQLTVIAVATSTPDRPQPPTAAYVQHHLGATGTAAFDVNAVCSGTVFALSSVAGTLVY |
| | (Streptomyces sp. st. | RGGYALVIGADLYSRILNPADRKTVVLFGDGAGAMVLGPTSTGTGPIVRRVALHTFGGLTDLIRVPA |
| | CL190) UniProt ID: | GGSRQPLDTDGLDAGLQYFAMDGREVRRFVTEHLPQLIKGFLHEAGVDAADISHFVPHQANGVM |
| | D7URV0-1 | LDEVFGELHLPRATMHRTVETYGNTGAASIPITMDAAVRAGSFRPGELVLLAGFGGGMAASFALIE |
| | | W |
| 26 | Clostridial 3- | MKKVCVIGAGTMGSGIAQAFAAKGFEVVLRDIKDEFVDRGLDFINKNLSKLVKKGKIEEATKVEILTR |
| | hydroxybutyryl-CoA | ISGTVDLNMAADCDLVIEAAVERMDIKKQIFADLDNICKPETILASNTSSLSITEVASATKRPDKVIGM |
| | dehydrogenase Hbd | HFFNPAPVMKLVEVIRGIATSQETFDAVKETSIAIGKDPVEVAEAPGFVVNRILIPMINEAVGILAEGI |
| | (Clostridium | ASVEDIDKAMKLGANHPMGPLELGDFIGLDICLAIMDVLYSETGDSKYRPHTLLKKYVRAGWLGRKS |
| | acetobutylicum ATCC | GKGFYDYSK |
| | 824), Genbank ID: | |
| | P52041 | |
| 27 | Crt | MELNNVILEKEGKVAVVTINRPKALNALNSDTLKEMDYVIGEIENDSEVLAVILTGAGEKSFVAGADI |
| | (C. acetobutylicum | SEMKEMNTIEGRKFGILGNKVFRRLELLEKPVIAAVNGFALGGGCEIAMSCDIRIASSNARFGQPEV |
| | ATCC 824), Genbank | GLGITPGFGGTQRLSRLVGMGMAKQLIFTAQNIKADEALRIGLVNKVVEPSELMNTAKEIANKIVSN |
| | ID: P52046 | APVAVKLSKQAINRGMQCDIDTALAFESEAFGECFSTEDQKDAMTAFIEKRKIEGFKNR |
| 28 | Ter (Treponema | MIVKPMVRNNICLNAHPQGCKKGVEDQIEYTKKRITAEVKAGAKAPKNVLVLGCSNGYGLASRITA |
| | denticola ATCC | AFGYGAATIGVSFEKAGSETKYGTPGWYNNLAFDEAAKREGLYSVTIDGDAFSDEIKAQVIEEAKK |
| | 35405), Genbank ID: | KGIKFDLIVYSLASPVRTDPDTGIMHKSVLKPFGKTFTGKTVDPFTGELKEISAEPANDEEAAATVKV |
| | Q73Q47 | MGGEDWERWIKQLSKEGLLEEGCITLAYSYIGPEATQALYRKGTIGKAKEHLEATAHRLNKENPSI |
| | | RAFVSVNKGLVTRASAVIPVIPLYLASLFKVMKEKGNHEGCIEQITRLYAERLYRKDGTIPVDEENRI |
| | | RIDDWELEEDVQKAVSALMEKVTGENAESLTDLAGYRHDFLASNGFDVEGINYEAEVERFDRI |
| 29 | YciA (Haemophilus | MSANFTDKNGRQSKGVLLLRTLAMPSDTNANGDIFGGWIMSQMDMGGAILAKEIAHGRVVTVAVE |
| | influenza), Genbank | SMNFIKPISVGDVVCCYGQCLKVGRSSIKIKVEVWVKKVASEPIGERYCVTDAVFTFVAVDNNGRS |
| | ID: AAC22485 | RTIPRENNQELEKALALISEQPL |
| 30 | Linalool synthase | MQEFEFAVPAPSRVSPDLARARARHLDWVHAMDLVRGEEARRRYEFSCVADIGAYGYPHATGAD |
| | from Streptomyces | LDLCVDVLGWTFLFDDQFDAGDGRERDALAVCAELTDLLWKGTAATAASPPIVVAFSDCWERMR |
| | clavuligerus | AGMSDAWRRRTVHEWVDYLAGWPTKLADRAHGAVLDPAAHLRARHRTICCRPLFALAERVGGY |
| | (UniProtKB-D5SL78) | EVPRRAWHSSRLDGMRFTTSDAVIGMNELHSFEKDRAQGHANLVLSLVHHGGLTGPEAVTRVCD |
| | | LVQGSIESFLRLRSGLPELGRALGVEGAVLDRYADALSAFCRGYHDWGRGASRYTTRDHPGDLG |
| | | LENLVARSSG |
| 31 | Linalool synthase | MQEFEFAVPAPSRVSPDLARARARHLDWVHAMDLVRGEEARRRYEFSCVADIGAYGYPHATGAD |
| | variant bLinS-L72M | LDLCVDVMGWTFLFDDQFDAGDGRERDALAVCAELTDLLWKGTAATAASPPIVVAFSDCWERMR |
| | | AGMSDAWRRRTVHEWVDYLAGWPTKLADRAHGAVLDPAAHLRARHRTICCRPLFALAERVGGY |
| | | EVPRRAWHSSRLDGMRFTTSDAVIGMNELHSFEKDRAQGHANLVLSLVHHGGLTGPEAVTRVCD |
| | | LVQGSIESFLRLRSGLPELGRALGVEGAVLDRYADALSAFCRGYHDWGRGASRYTTRDHPGDLG |
| | | LENLVARSSG |
| 32 | Linalool synthase | MQEFEFAVPAPSRVSPDLARARARHLDWVHAMDLVRGEEARRRYEFSCVADIGAYGYPHATGAD |
| | variant bLinS-V214L | LDLCVDVLGWTFLFDDQFDAGDGRERDALAVCAELTDLLWKGTAATAASPPIVVAFSDCWERMR |
| | | AGMSDAWRRRTVHEWVDYLAGWPTKLADRAHGAVLDPAAHLRARHRTICCRPLFALAERVGGY |
| | | EVPRRAWHSSRLDGMRFTTSDALIGMNELHSFEKDRAQGHANLVLSLVHHGGLTGPEAVTRVCD |
| | | LVQGSIESFLRLRSGLPELGRALGVEGAVLDRYADALSAFCRGYHDWGRGASRYTTRDHPGDLG |
| | | LENLVARSSG |
| 33 | Linalool synthase | MQEFEFAVPAPSRVSPDLARARARHLDWVHAMDLVRGEEARRRYEFSCVADIGAYGYPHATGAD |
| | variant bLinS-V2141 | LDLCVDVLGWTFLFDDQFDAGDGRERDALAVCAELTDLLWKGTAATAASPPIVVAFSDCWERMR |
| | | AGMSDAWRRRTVHEWVDYLAGWPTKLADRAHGAVLDPAAHLRARHRTICCRPLFALAERVGGY |
| | | EVPRRAWHSSRLDGMRFTTSDAIIGMNELHSFEKDRAQGHANLVLSLVHHGGLTGPEAVTRVCDL |
| | | VQGSIESFLRLRSGLPELGRALGVEGAVLDRYADALSAFCRGYHDWGRGASRYTTRDHPGDLGL |
| | | ENLVARSSG |
| 34 | First 16S genomic | aggagatataccatgCCTACGGGNGGCWGCAG |
| | hypervariable region | |
| 35 | Second 16S genomic | tggtggtgctcgagGACTACGGGTATCTAATCC |
| | hypervariable region | |
| 36 | PCR primer 1 | CTCGAGCACCACCACCACC |
| 37 | PCR primer 2 | CTCTAGAAATAATTTTGTTTAACTTTAAGAAGG-AGATATACCatg |
| 38 | Ferredoxin (PetF) | MASYTVKLITPDGESSIECSDDTYILDAAEEAGLDLPYSCRAGACSTCAGKITAGSVDQSDQSFLD |
| | from Synechocystis | DDQIEAGYVLTCVAYPTSDCTIETHKEEDLY |
| | sp. PCC6803 | |
| 39 | Full length Abies | MAYSAMATMGYNGMAASCHTLHPTSPLKPFHGASTSLEAFNGEHMGLLRGYSKRKLSSYKNPAS |
| | grandis GPPS | RSSNATVAQLLNPPQKGKKAVEFDFNKYMDSKAMTVNEALNKAIPLRYPQKIYESMRYSLLAGGK |
| | sequence (GenBank: | RVRPVLCIAACELVGGTEELAIPTACAIEMIHTMSLMHDDLPCIDNDDLRRGKPTNHKIFGEDTAVT |
| | AAN01134.1) | AGNALHSYAFEHIAVSTSKTVGADRILRMVSELGRATGSEGVMGGQMVDIASEGDPSIDLQTLEWI |
| | | HIHKTAMLLECSVVCGAIIGGASEIVIERARRYARCVGLLFQVVDDILDVTKSSDELGKTAGKDLISD |
| | | KATYPKLMGLEKAKEFSDELLNRAKGELSCFDPVKAAPLLGLADYVAFRQN |
| 40 | Truncated GPPS | MEFDFNKYMDSKAMTVNEALNKAIPLRYPQKIYESMRYSLLAGGKRVRPVLCIAACELVGGTEELA |
| | sequence | IPTACAIEMIHTMSLMHDDLPCIDNDDLRRGKPTNHKIFGEDTAVTAGNALHSYAFEHIAVSTSKTV |
| | | GADRILRMVSELGRATGSEGVMGGQMVDIASEGDPSIDLQTLEWIHIHKTAMLLECSVVCGAIIGG |
| | | ASEIVIERARRYARCVGLLFQVVDDILDVTKSSDELGKTAGKDLISDKATYPKLMGLEKAKEFSDELL |
| | | NRAKGELSCFDPVKAAPLLGLADYVAFRQN |
| 41 | Carboxylic acid | MSPITREERLERRIQDLYANDPQFAAAKPATAITAAIERPGLPLPQIIETVMTGYADRPALAQRSVEF |
| | reductase from | VTDAGTGHTTLRLLPHFETISYGELWDRISALADVLSTEQTVKPGDRVCLLGFNSVDYATIDMTLAR |
| | Mycobacterium | LGAVAVPLQTSAAITQLQPIVAETQPTMIAASVDALADATELALSGQTATRVLVFDHHRQVDAHRAA |
| | marinum | VESARERLAGSAVVETLAEAIARGDVPRGASAGSAPGTDVSDDSLALLIYTSGSTGAPKGAMYPR |
| | | RNVATFWRKRTWFEGGYEPSITLNFMPMSHVMGRQILYGTLQNGGTAYFVAKSDLSTLFEDLALV |
| | | RPTELTFVPRVWDMVFDEFQSEVDRRLVDGADRVALEAQVKAEIRNDVLGGRYTSALTGSAPISD |
| | | EMKAWVEELLDMHLVEGYGSTEAGMILIDGAIRRPAVLDYKLVDVPDLGYFLTDRPHPRGELLVKT |
| | | DSLFPGYYQRAEVTADVFDADGFYRTGDIMAEVGPEQFVYLDRRNNVLKLSQGEFVTVSKLEAVF |
| | | GDSPLVRQIYIYGNSARAYLLAVIVPTQEALDAVPVEELKARLGDSLQEVAKAAGLQSYEIPRDFIIE |
| | | TTPWTLENGLLTGIRKLARPQLKKHYGELLEQIYTDLAHGQADELRSLRQSGADAPVLVTVCRAAA |
| | | ALLGGSASDVQPDAHFTDLGGDSLSALSFTNLLHEIFDIEVPVGVIVSPANDLQALADYVEAARKPG |
| | | SSRPTFASVHGASNGQVTEVHAGDLSLDKFIDAATLAEAPRLPAANTQVRTVLLTGATGFLGRYLA |
| | | LEWLERMDLVDGKLICLVRAKSDTEARARLDKTFDSGDPELLAHYRALAGDHLEVLAGDKGEADL |
| | | GLDRQTWQRLADTVDLIVDPAALVNHVLPYSQLFGPNALGTAELLRLALTSKIKPYSYTSTIGVADQ |
| | | IPPSAFTEDADIRVISATRAVDDSYANGYSNSKWAGEVLLREAHDLCGLPVAVFRCDMILADTTWA |
| | | GQLNVPDMFTRMILSLAATGIAPGSFYELAADGARQRAHYDGLPVEFIAEAISTLGAQSQDGFHTY |
| | | HVMNPYDDGIGLDEFVDWLNESGCPIQRIADYGDWLQRFETALRALPDRQRHSSLLPLLHNYRQP |
| | | ERPVRGSIAPTDRFRAAVQEAKIGPDKDIPHVGAPIIVKYVSDLRLLGLL |
| 42 | PPTase/Sfp | ATGAAGATTTACGGAATTTATATGGACCGCCCGCTTTCACAGGAAGAAAATGAACGGTTCATG |
| | (maturation factor | TCTTTCATATCACCTGAAAAACGGGAGAAATGCCGGAGATTTTATCATAAAGAAGATGCTCACC |
| | phosphopantetheinyl | GCACCCTGCTGGGAGATGTGCTCGTTCGCTCAGTCATAAGCAGGCAGTATCAGTTGGACAAA |
| | transferase) from | TCCGATATCCGCTTTAGCACGCAGGAATACGGGAAGCCGTGCATCCCTGATCTTCCCGACGC |
| | Bacillus subtilis | TCATTTCAACATTTCTCACTCCGGACGCTGGGTCATTTGCGCGTTTGATTCACAGCCGATCGG |
| | (Genbank: X65610.1) | CATAGATATCGAAAAAACGAAACCGATCAGCCTTGAGATGCCCAAGCGCTTCTTTTCAAAAAC |
| | | AGAGTACAGCGACCTTTTAGCAAAAGACAAGGACGAGCAGACAGACTATTTTTATCATCTATG |
| | | GTCAATGAAAGAAAGCTTTATCAAACAAGGAAGGCAAAGGCTTATCGCTTCCGCTTGATTCCTT |
| | | TTCAGTGCGCCTGCACCAGGACGGACAAGTATCCATTGAGCTTCCGGACAGCCATTCCCCAT |
| | | GCTATATCAAAACGTATGAGGTCGATCCCGGCTACAAAATGGCTGTATGCGCCGTACACCCTG |
| | | ATTTCCCCGAGGATATCACAATGGTCTCGTACGAAGAGCTTTTATA |
| 43 | Nocardia iowensis | MAVDSPDERLQRRIAQLFAEDEQVKAARPLEAVSAAVSAPGMRLAQIAATVMAGYADRPAAGQR |
| | CAR (UniProt: | AFELNTDDATGRTSLRLLPRFETITYRELWQRVGEVAAAWHHDPENPLRAGDFVALLGFTSIDYAT |
| | Q6RKB1) | LDLADIHLGAVTVPLQASAAVSQLIAILTETSPRLLASTPEHLDAAVECLLAGTTPERLVVFDYHPED |
| | | DDQRAAFESARRRLADAGSLVIVETLDAVRARGRDLPAAPLFVPDTDDDPLALLIYTSGSTGTPKG |
| | | AMYTNRLAATMWQGNSMLQGNSQRVGINLNYMPMSHIAGRISLFGVLARGGTAYFAAKSDMSTL |
| | | FEDIGLVRPTEIFFVPRVCDMVFQRYQSELDRRSVAGADLDTLDREVKADLRQNYLGGRFLVAVV |
| | | GSAPLAAEMKTFMESVLDLPLHDGYGSTEAGASVLLDNQIQRPPVLDYKLVDVPELGYFRTDRPH |
| | | PRGELLLKAETTIPGYYKRPEVTAEIFDEDGFYKTGDIVAELEHDRLVYVDRRNNVLKLSQGEFVTV |
| | | AHLEAVFASSPLIRQIFIYGSSERSYLLAVIVPTDDALRGRDTATLKSALAESIQRIAKDANLQPYEIP |
| | | RDFLIETEPFTIANGLLSGIAKLLRPNLKERYGAQLEQMYTDLATGQADELLALRREAADLPVLETVS |
| | | RAAKAMLGVASADMRPDAHFTDLGGDSLSALSFSNLLHEIFGVEVPVGVVVSPANELRDLANYIEA |
| | | ERNSGAKRPTFTSVHGGGSEIRAADLTLDKFIDARTLAAADSIPHAPVPAQTVLLTGANGYLGRFLC |
| | | LEWLERLDKTGGTLICVVRGSDAAAARKRLDSAFDSGDPGLLEHYQQLAARTLEVLAGDIGDPNL |
| | | GLDDATWQRLAETVDLIVHPAALVNHVLPYTQLFGPNVVGTAEIVRLAITARRKPVTYLSTVGVADQ |
| | | VDPAEYQEDSDVREMSAVRVVRESYANGYGNSKWAGEVLLREAHDLCGLPVAVFRSDMILAHSR |
| | | YAGQLNVQDVFTRLILSLVATGIAPYSFYRTDADGNRQRAHYDGLPADFTAAAITALGIQATEGFRT |
| | | YDVLNPYDDGISLDEFVDWLVESGHPIQRITDYSDWFHRFETAIRALPEKQRQASVLPLLDAYRNP |
| | | CPAVRGAILPAKEFQAAVQTAKIGPEQDIPHLSAPLIDKYVSDLELLQLL |
| 44 | Mycobacterium phlei | MASESRDVRLQRRIAELYDTDPQFAAARPDEAVARAVNAPGLTLSQVIRTVLDNYADRPALGYRA |
| | CAR (UniProt: | VEFAADPASARTVARLLPRFDTITYRELGERIDATTAALGHDGLRPGERVAILGFSSVDYTTIDIAAF |
| | A0A5N5VEC4) | NLGAVSVPLQTSAPPSQLRPMTAETEPAVIAASVDFLDDALELIRTGHAPRRLVVFDFHPEIDDHRD |
| | | ALAAATTGLADTAMTVETLDSLLTRGRTLPAPTGYRDRDDDLALLIYTSGSTGAPKGAMYQRRMV |
| | | TNMWRRATTAIWGGKEAAPWLTLNFMPMSHVMGRGILSTTLCSGGTAYFAARSDLSTLLEDLALV |
| | | RPTQLTFVPRIWEMIFQEYQRDVARRPEAEVLADLREHRLGGRFLAAMTGSAPMSPEMTAFAESV |
| | | LDLHLVDGYGSTEAGSIVLDGQVLRPPVLDYKLVDVPELGYFSTDRPYPRGELLVKTELMFPGYYK |
| | | RPDITAEVFDEDGYYRTGDIVAELGPDRLAYVDRRNNVLKLSQGEFVTVSKLEAAFAASPLVRQIYI |
| | | YGNSAHPYLLAVVVPTEDALTRYDAATLKTAISESLQDVGRAAGLQSYEIPRDFLVETTPFTLENGL |
| | | LTGLRKLARPKLKEHYGERLEQLYTELADGQAEELKELRTHGAQQPTLTTVSRAATALLGTASAEV |
| | | RPDAHFTDLGGDSLSALTFGNLLGEIYAVEVPVGVIVSPANDLAAIADYIDTARRPGDGRPTFAGVH |
| | | GDDAAEVHARDLTLDRFLDDATLAAATALPGPAPEIRTVLLTGATGFLGRYLALEWLERMAMVGG |
| | | TLICLVRGRDDAAARARLDQIFDSGDPELLRHYRELADRHLEVIAGDKSDAVLGLDRRTWQRLADT |
| | | VDLIVDPAALVNHVLPYRELFGPNVVGTAELIRLALTGRLKPYLYTSTIAVGAGIAPGQFTEDADIRQI |
| | | SATRTLDDSYANGYATSKWAGEVLLREAHDLCGLPVAVFRCDMILADTSYAGQLNVPDMFTRLILS |
| | | LVATGIAPLSFYELDAAGHRQRAHYDGLPVEFVAEAVSALGLDVAEDGGFATYHVMNPYDDGIGL |
| | | DEFVDWLTGAGYPIERVNDYGTWFQRFETAIRGLPERQRQASLLPLLHNYQRPQPPIRGSAAPTD |
| | | RFRSAVQDAKIGPDKDIPHITPEVIVKYVTDLRLLGLL |
| 45 | Mycobacterium | MTIETREDRFNRRIDHLFETDPQFAAARPDEAISAAAADPELRLPAAVKQILAGYADRPALGKRAVE |
| | smegmatis CAR1 | FVTDEEGRTTAKLLPRFDTITYRQLAGRIQAVTNAWHNHPVNAGDRVAILGFTSVDYTTIDIALLELG |
| | (UniProt: | AVSVPLQTSAPVAQLQPIVAETEPKVIASSVDFLADAVALVESGPAPSRLVVFDYSHEVDDQREAF |
| | A0A653FAQ0) | EAAKGKLAGTGVVVETITDALDRGRSLADAPLYVPDEADPLTLLIYTSGSTGTPKGAMYPESKTAT |
| | | MWQAGSKARWDETLGVMPSITLNFMPMSHVMGRGILCSTLASGGTAYFAARSDLSTFLEDLALV |
| | | RPTQLNFVPRIWDMLFQEYQSRLDNRRAEGSEDRAEAAVLEEVRTQLLGGRFVSALTGSAPISAE |
| | | MKSWVEDLLDMHLLEGYGSTEAGAVFIDGQIQRPPVIDYKLVDVPDLGYFATDRPYPRGELLVKSE |
| | | QMFPGYYKRPEITAEMFDEDGYYRTGDIVAELGPDHLEYLDRRNNVLKLSQGEFVTVSKLEAVFG |
| | | DSPLVRQIYVYGNSARSYLLAVVVPTEEALSRWDGDELKSRISDSLQDAARAAGLQSYEIPRDFLV |
| | | ETTPFTLENGLLTGIRKLARPKLKAHYGERLEQLYTDLAEGQANELRELRRNGADRPVVETVSRAA |
| | | VALLGASVTDLRSDAHFTDLGGDSLSALSFSNLLHEIFDVDVPVGVIVSPATDLAGVAAYIEGELRG |
| | | SKRPTYASVHGRDATEVRARDLALGKFIDAKTLSAAPGLPRSGTEIRTVLLTGATGFLGRYLALEW |
| | | LERMDLVDGKVICLVRARSDDEARARLDATFDTGDATLLEHYRALAADHLEVIAGDKGEADLGLDH |
| | | DTWQRLADTVDLIVDPAALVNHVLPYSQMFGPNALGTAELIRIALTTTIKPYVYVSTIGVGQGISPEA |
| | | FVEDADIREISATRRVDDSYANGYGNSKWAGEVLLREAHDWCGLPVSVFRCDMILADTTYSGQLN |
| | | LPDMFTRLMLSLVATGIAPGSFYELDADGNRQRAHYDGLPVEFIAEAISTIGSQVTDGFETFHVMN |
| | | PYDDGIGLDEYVDWLIEAGYPVHRVDDYATWLSRFETALRALPERQRQASLLPLLHNYQQPSPPV |
| | | CGAMAPTDRFRAAVQDAKIGPDKDIPHVTADVIVKYISNLQMLGLL |
| 46 | Mycobacterium | MTSDVHDATDGVTETALDDEQSTRRIAELYATDPEFAAAAPLPAVVDAAHKPGLRLAEILQTLFTG |
| | smegmatis CAR2 | YGDRPALGYRARELATDEGGRTVTRLLPRFDTLTYAQVWSRVQAVAAALRHNFAQPIYPGDAVAT |
| | (UniProt: | IGFASPDYLTLDLVCAYLGLVSVPLQHNAPVSRLAPILAEVEPRILTVSAEYLDLAVESVRDVNSVSQ |
| | A0A653FCI4) | LVVFDHHPEVDDHRDALARAREQLAGKGIAVTTLDAIADEGAGLPAEPIYTADHDQRLAMILYTSGS |
| | | TGAPKGAMYTEAMVARLWTMSFITGDPTPVINVNFMPLNHLGGRIPISTAVQNGGTSYFVPESDM |
| | | STLFEDLALVRPTELGLVPRVADMLYQHHLATVDRLVTQGADELTAEKQAGAELREQVLGGRVITG |
| | | FVSTAPLAAEMRAFLDITLGAHIVDGYGLTETGAVTRDGVIVRPPVIDYKLIDVPELGYFSTDKPYPR |
| | | GELLVRSQTLTPGYYKRPEVTASVFDRDGYYHTGDVMAETAPDHLVYVDRRNNVLKLAQGEFVA |
| | | VANLEAVESGAALVRQIFVYGNSERSFLLAVVVPTPEALEQYDPAALKAALADSLQRTARDAELQS |
| | | YEVPADFIVETEPFSAANGLLSGVGKLLRPNLKDRYGQRLEQMYADIAATQANQLRELRRAAATQP |
| | | VIDTLTQAAATILGTGSEVASDAHFTDLGGDSLSALTLSNLLSDFFGFEVPVGTIVNPATNLAQLAQ |
| | | HIEAQRTAGDRRPSFTTVHGADATEIRASELTLDKFIDAETLRAAPGLPKVTTEPRTVLLSGANGWL |
| | | GRFLTLQWLERLAPVGGTLITIVRGRDDAAARARLTQAYDTDPELSRRFAELADRHLRVVAGDIGD |
| | | PNLGLTPEIWHRLAAEVDLVVHPAALVNHVLPYRQLFGPNVVGTAEVIKLALTERIKPVTYLSTVSV |
| | | AMGIPDFEEDGDIRTVSPVRPLDGGYANGYGNSKWAGEVLLREAHDLCGLPVATFRSDMILAHPR |
| | | YRGQVNVPDMFTRLLLSLLITGVAPRSFYIGDGERPRAHYPGLTVDFVAEAVTTLGAQQREGYVSY |
| | | DVMNPHDDGISLDVFVDWLIRAGHPIDRVDDYDDWVRRFETALTALPEKRRAQTVLPLLHAFRAP |
| | | QAPLRGAPEPTEVFHAAVRTAKVGPGDIPHLDEALIDKYIRDLREFGLI |
| 47 | Nocardia | MLDDARAERRERRIADALADDQVREAVADAAVSESVRRVEVRLARIVDAVMSGYGDRAALAWRR |
| | otitidiscaviarum CAR | SELVDGAVRLLPEYSTMTYRELWRRAGAVAAEWGADAESPVRAGDFVCTLGFTSPDYTVVDLAL |
| | (UniProt: | VRLGAVAVPLQASASVAQWRSIMAETEPRMLATSAETLPAAVEAVLGGFAPRRVLVFDYRPELEA |
| | A0A516NPD0) | HRSAVDSARERLAEVGCRVATVADAVDRGASLPAPLCIPSDRERLALLIYTSGSTGAPKGAMYTD |
| | | RLAAGLWLSANDIRVPALTMNYMPLSHVAGRMSLYGTLMRGGTAYFAAASDMSTLLDDFGLARPT |
| | | ELFLVPRVCELLHQRYQSELDRRVLAGDDVETTATDVKAELRERVLGGRYLTALSGSAPLAAEMK |
| | | TFMESLLDDELHDGYGSTEAGGSVLLDNRIKRPPVLDYRLVDVPELGYFRTDKPHPRGELLLKTES |
| | | MFPGYYKRPEITAEMFDADGFYRTGDVVAELGPDQLVYVDRRNNVLKLSQGEFVTVAALEAVYAT |
| | | SPLIRQIFVYGSSERAYLLAVVVPTDEVLALPPARARAEVGESLHRIAKESGLQPYEIPREVLIESEP |
| | | FTIDNGLLSGIGKLLRPKLKEHYGEQLERLYTELAQQREDELTTLRRGAHERPVLETVTRAAGAVLD |
| | | LAAGEVSPDAHFADLGGDSLSALSFSTLLRDIFGVEVPVGVIVGPATDLTRIAEYLVAERDSGTRPT |
| | | AATVHGDDGLLRADDLTLEAFLDPGTLAAATRLSAPTDPPRTVLLTGANGYLGRFLALEWLQRLDV |
| | | SGGTLICLVRGSDADAARRRLDAVFATGDPELEAHYRDLAGRRLRVLAGDIGEPNLGLSEQDWRD |
| | | LAESVDVIVHPAALVNHVLPYAQLFGPNVVGTAEVIRLALTARLKPVTYLSTVAVSAGIDPETFTEDG |
| | | DIREISPVRRLDDGYANGYGNSKWAGEVLLRAAHDRFGLPVAVFRSDMILAHSHYAGQLNVPDMF |
| | | TRLLLSVLATGLAPGSFHDAHGERHRAHYDGLPADFTAAAVTTLGAQATSGFDTYDVLNPHDDGL |
| | | SLDTFIDWLIDSGHPIDRIDDYADWFTRFETAVRALPEPQRQHSLLPLLHAYRHPTPPLRGAALPAK |
| | | HFRAAVQQAKLGPDADIPHLTRELIEKYVSDLRLLGLLG |
| 48 | Tsukamurella | MSIETVQNGVPAEGSVPPADQQTERLPQVIARIFAQFADRPAFATREAGPGTPYATVSYREIWRRV |
| | paurometabola CAR | TALVASWQSEVAPGDFVAILGFTSSDFVTVDLATTLLGAPNVPLQAGAPAARIATILDETRPKILAVS |
| | (UniProt: D5UWX9) | ADQVDLAQEALAESAATPRVVVFDGERDGYEGIEADILSGSALPAPEFFAPEPGTDPLVTLIYTSGS |
| | | TGTPKGAMYTEQLVRDAWLKVDSIVDIDMPAESLLHFLPMSHMYGRNWLIAGLASGGTGYFAGAS |
| | | DMSTLFDDLAAARPTAIGLVPRVCELIHQRYLAVEADTDAETARVELRDRVLGGRLQAAMCGSAAL |
| | | SSELQTFMEWLLGIDIQIGYGSTEAGGVIRDGVVVRPPVTEYKLIDVPELGYFVTDSPHPRGELLVK |
| | | STQLIPGYYNSDKRIRDDEGFYRTGDVMAELGPDRLEYVDRRSNVIKLAQGEFVPIAQLEAIYAAGP |
| | | DVHQIFLYGTSERSYLIGVVVPAPGPDGETDAQTRTRVLDGLAAIARENDLAAYEVPRDVLIERDPF |
| | | SQENGLRSGIGKLVRPALIARYGDRLHDLYAQADTRQREGLRALDASGPIIDTVLGAAALTLGADIA |
| | | DFDADTRFGDLGGDSLSALSLATTLEGLYDVPVPVQTIVGPTATLGGVARHIEKARSGGVAAPTAD |
| | | SVHGVGASVARATDLTLEKFIDPELLALAPTLPAATGEPNTVLLTGSTGYLGRFLLLDWLRRVAPH |
| | | GGTVIALVRGADADDARRRVTAAIGDSDPDLTQEFTSLAEHHLHVIAGDFGSPALGLDDATWSDLA |
| | | GRVDHVVHCGALVNHVLPYDQLFGPNVVATGEVVRLALTTRRKSVDYVSTVAVVPQDDGRVLVE |
| | | DDDVRELGAERRIGADAYANGYAVSKWAGEVLLHEAADLADLPVRVFRSDMILAHSRFHGQFNEV |
| | | DQFTRLLLSIAETGLAPASFYTPDPSGHRPHYDGLPVDFTAEAITTLSAAGRSGYRTFHVLNANDD |
| | | GVSLDSFVDWIAASGRSIERIDDYDTWFARFEQALQQLPDEARQRSVLPLLHAVREPAPAAGTSAL |
| | | SVDRFRGAVRETGVGPGDIPVLDRALIEKYLRDFETAGWLAPGARD |
| 49 | Chromobacterium | MQKQRTTSQWRELDAAHHLHPFTDTASLNQAGARVMTRGEGVYLWDSEGNKIIDGMAGLWCVN |
| | violaceum TA | VGYGRKDFAEAARRQMEELPFYNTFFKTTHPAVVELSSLLAEVTPAGFDRVFYTNSGSESVDTMI |
| | (UniProt: Q7NWG4) | RMVRRYWDVQGKPEKKTLIGRWNGYHGSTIGGASLGGMKYMHEQGDLPIPGMAHIEQPWWYKH |
| | | GKDMTPDEFGVVAARWLEEKILEIGADKVAAFVGEPIQGAGGVIVPPATYWPEIERICRKYDVLLVA |
| | | DEVICGFGRTGEWFGHQHFGFQPDLFTAAKGLSSGYLPIGAVFVGKRVAEGLIAGGDFNHGFTYS |
| | | GHPVCAAVAHANVAALRDEGIVQRVKDDIGPYMQKRWRETFSRFEHVDDVRGVGMVQAFTLVKN |
| | | KAKRELFPDFGEIGTLCRDIFFRNNLIMRACGDHIVSAPPLVMTRAEVDEMLAVAERCLEEFEQTLK |
| | | ARGLA |
| 50 | Vibrio fluvialis TA | MNKPQSWEARAETYSLYGFTDMPSLHQRGTVVVTHGEGPYIVDVNGRRYLDANSGLWNMVAGF |
| | (UniProt: F2XBU9) | DHKGLIDAAKAQYERFPGYHAFFGRMSDQTVMLSEKLVEVSPFDSGRVFYTNSGSEANDTMVKM |
| | | LWFLHAAEGKPQKRKILTRWNAYHGVTAVSASMTGKPYNSVFGLPLPGFVHLTCPHYWRYGEEG |
| | | ETEEQFVARLARELEETIQREGADTIAGFFAEPVMGAGGVIPPAKGYFQAILPILRKYDIPVISDEVIC |
| | | GFGRTGNTWGCVTYDFTPDAIISSKNLTAGFFPMGAVILGPELSKRLETAIEAIEEFPHGFTASGHP |
| | | VGCAIALKAIDVVMNEGLAENVRRLAPRFEERLKHIAERPNIGEYRGIGFMWALEAVKDKASKTPFD |
| | | GNLSVSERIANTCTDLGLICRPLGQSVVLCPPFILTEAQMDEMFDKLEKALDKVFAEVA |
| 51 | Alcaligenes | MSAAKLPDLSHLWMPFTANRQFKANPRLLASAKGMYYTSFDGRQILDGTAGLWCVNAGHCREEI |
| | denitrificans TA | VSAIASQAGVMDYAPGFQLGHPLAFEAATAVAGLMPQGLDRVFFTNSGSESVDTALKIALAYHRA |
| | (UniProt: Q7WWK8) | RGEAQRTRLIGRERGYHGVGFGGISVGGISPNRKTFSGALLPAVDHLPHTHSLEHNAFTRGQPEW |
| | | GAHLADELERIIALHDASTIAAVIVEPMAGSTGVLVPPKGYLEKLREITARHGILLIFDEVITAYGRLG |
| | | ATAAAYFGVTPDLITMAKGVSNAAVPAGAVAVRREVHDAIVNGPQGGIEFFHGYTYSAHPLAAAAV |
| | | ELATLDIYRREDLFARARKLSAAFEEAAHSLKGAPHVIDVRNIGLVAGIELSPREGAPGARAAEAFQK |
| | | CFDTGLMVRYTGDILAVSPPLIVDENQIGQIFEGIGKVLKEVA |
| 52 | Pseudomonas putida | MSEQNSQTLAWQTMSRDHHLAPFSDVRQLAEKGPRIITSAKGVYLWDSEGNKILDGMAGLWCVA |
| | TA (UniProt: Q88KV9) | VGYGREELAEVASQQMKQLPYYNLFFQTAHPPALELAKAIAEVAPQGMNHVFFTGSGSEGNDTVL |
| | | RMVRHYWALKGQKNKKVIIGRINGYHGSTVAGAALGGMSGMHQQGGVIPDVVHIPQPYWFGEGG |
| | | DMTEADFGVWAAEQLEKKILEVGVDNVAAFIAEPIQGAGGVIIPPQTYWPKIKEILARYDILFVADEVI |
| | | CGFGRTGEWFGTDYYDLKPDLMTIAKGLTSGYIPMGGVIVRDEVAKVISEGGDFNHGFTYSGHPV |
| | | AAAVGLENLRILRDEQIIQQVHDKTAPYLQQRLRELADHPLVGEVRGLGMLGAIELVKDKATRARH |
| | | EGKGVGMICRQHCFDNGLIMRAVGDTMIIAPPLVISIEEIDELVEKARKCLDLTYEAVR |
| 53 | Paracoccus | MNQPQSWEARAETYSLYGFTDMPSVHQRGTVVVTHGEGPYIVDVHGRRYLDANSGLWNMVAGF |
| | denitrificans TA | DHKGLIEAAKAQYDRFPGYHAFFGRMSDQTVMLSEKLVEVSPFDNGRVFYTNSGSEANDTMVKM |
| | (UniProt: A1B956) | LWFLHAAEGKPQKRKILTRWNAYHGVTAVSASMTGKPYNSVFGLPLPGFIHLTCPHYWRYGEEG |
| | | ETEAQFVARLARELEDTITREGADTIAGFFAEPVMGAGGVIPPAKGYFQAILPILRKYDIPMISDEVIC |
| | | GFGRTGNTWGCLTYDFMPDAIISSKNLTAGFFPMGAVILGPDLAKRVEAAVEAIEEFPHGFTASGH |
| | | PVGCAIALKAIDVVMNEGLAENVRRLAPRFEAGLKRIADRPNIGEYRGIGFMWALEAVKDKPTKTPF |
| | | DANLSVSERIANTCTDLGLICRPLGQSIVLCPPFILTEAQMDEMFEKLEKALDKVFAEVA |
| 54 | PaTA UNIPROT: | MNSQITNAKTREWQALSRDHHLPPFTDYKQLNEKGARIITKAEGVYIWDSEGNKILDAMAGLWCV |
| | Q916J2 amino acid | NVGYGREELVQAATRQMRELPFYNLFFQTAHPPVVELAKAIADVAPEGMNHVFFTGSGSEANDTV |
| | sequence | LRMVRHYWATKGQPQKKVVIGRWNGYHGSTVAGVSLGGMKALHEQGDFPIPGIVHIAQPYWYGE |
| | | GGDMSPDEFGVWAAEQLEKKILEVGEENVAAFIAEPIQGAGGVIVPPDTYWPKIREILAKYDILFIAD |
| | | EVICGFGRTGEWFGSQYYGNAPDLMPIAKGLTSGYIPMGGVVVRDEIVEVLNQGGEFYHGFTYSG |
| | | HPVAAAVALENIRILREEKIIEKVKAETAPYLQKRWQELADHPLVGEARGVGMVAALELVKNKKTRE |
| | | RFTDKGVGMLCREHCFRNGLIMRAVGDTMIISPPLVIDPSQIDELITLARKCLDQTAAAVLA |
| 55 | Bacillus subtilis | MIIGVPKEIKNNENRVALTPGGVSQLISNGHRVLVETGAGLGSGFENEAYESAGAEIIADPKQVWD |
| | AlaDH | AEMVMKVKEPLPEEYVYFRKGLVLFTYLHLAAEPELAQALKDKGVTAIAYETVSEGRTLPLLTPMS |
| | (Uniprot: Q08352) | EVAGRMAAQIGAQFLEKPKGGKGILLAGVPGVSRGKVTIIGGGVVGTNAAKMAVGLGADVTIIDLN |
| | | ADRLRQLDDIFGHQIKTLISNPVNIADAVAEADLLICAVLIPGAKAPTLVTEEMVKQMKPGSVIVDVAI |
| | | DQGGIVETVDHITTHDQPTYEKHGVVHYAVANMPGAVPRTSTIALTNVTVPYALQIANKGAVKALA |
| | | DNTALRAGLNTANGHVTYEAVARDLGYEYVPAEKALQDESSVAGA |
| 56 | 6X His | HHHHHH |

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### EXAMPLES

### Example 1—Materials and Methods

### Materials, Plasmids and Strains

All chemicals and solvents were commercially sourced and were of analytical grade or better. Media components were purchased from Formedium (Norfolk, UK). Gene sequencing and oligonucleotide synthesis were performed by Eurofins MWG (Ebersberg, Germany) and Integrated DNA Technologies (Iowa, USA). Plasmids for propane production used in this study were pHALP7-CvFAP_{G462V} and pHALP102-CvFAP_{G462V} (FIG. 1) (Trisrivirat et al., 2020, https://doi.org/10.1093/synbio/ysaa022). These vectors contain the propane producing gene encoding fatty acid photodecarboxylase (CvFAP) from Chlorella variabilis NC64A variant G462V (Amer et al., 2020b). They also contain a broad-host-range origin of replication (oriV) from Pseudomonas aeruginosa, as well as an origin of transfer (oriT) and either a weak or strong constitutive promoter, respectively (Trisrivirat et al., 2020, https://doi.org/10.1093/synbio/ysaa022).

The E. coli strains S17-1 (Simon et al., 1983. Bio/Technology 1, 784-791) and BL21 (DE3) (New England Biolabs) were used for plasmid conjugation into Halomonas and radiolabelled CO₂ fixation assays, respectively. S. enterica and Halothiobacillus neapolitanus used for ¹⁴C fixation study were kindly supplied by Prof Liu (University of Liverpool, UK). Native H. bluephagenesis TD01 was previously isolated from the Aydingkol Lake in Xinjian, China (Tan et al., 2011. Bioresour. Technol. 102, 8130-8136). The engineered H. bluephagenesis strain TQ10 is a TD01 derivative that is lacking genes required for PHB production and has a genomic encoded T7-like MmP1 system for IPTG inducible recombinant protein expression (Amer et al., 2020a. Biotechnol. Biofuels 13, 125; Amer et al., 2020b. Energy Environ. Sci. 13, 1818-1831; Zhao et al., 2017. Metab. Eng. 39, 128-140). These strains were kindly supplied by Professor Guo-Qiang Chen (Tsinghua University, China).

Environmental water samples originating from a brine spring in Cheshire, UK were collected into sterile bottles and stored at 4° C. Brine (1 L) was filtered (0.2 μm) to remove debris and microorganisms. Multiple debris-free regions of the filter paper were sampled for microorganism content by touching with a sterile inoculation loop and resuspending in 10 mL sterilized brine filtrate. Serial dilutions (10, 100 and 1000-fold) of the samples in sterile brine filtrate were performed, and aliquots were cultivated on LB agar plates containing 5% (w/v) NaCl at 30° C. for several days until colonies appeared in the culture plates. Individual colonies were repeatedly picked and re-streaked on fresh plates to ensure isolate homogeneity. Glycerol stocks were generated of each isolate by combining equal volumes of overnight cultures with 50% sterile glycerol and flash freezing in liquid nitrogen.

### Growth Medium and Standard Cultivation

Standard cultivation of E. coli and Salmonella enterica subsp. enterica serovar Hillingdon ATCC9184 were performed in Luria broth (LB; 5 g/L yeast extract, 10 g/L tryptone and 10 g/L NaCl pH 7.0) at 37° C. for 16-24 h. High salt Luria broth (LB60; LB containing 60 g/L NaCl) was used routinely for heterotrophic cultivation of Halomonas strains at pH 6.8 (propane production) or 9.0 (conjugation and recombinant enzyme expression) at 30° C. Strain characterization studies were performed primarily in LB60 medium, varying the concentration of NaCl (1-20% (w/v)), pH (6-10) and supplemental butyric acid (20-80 mM). Growth of Halomonas strains was additionally performed in environmental water growth medium with or without supplemental glycerol (0.5% (w/v)). Environmental water was obtained from three sources within the Greater Manchester region (Medlock and Mersey rivers and the Rochdale canal; UK). This was subsequently filter purified and supplemented with NaCl to obtain a final 6% (w/v) concentration (by refractometer). An additional high salinity minimal medium was utilised for Halomonas cultivation consisting of filter sterilised sea water (Irish sea) with or without a supplemental carbon source (0.5% (w/v) glycerol).

Chemoautotrophic growth of new isolates and control strains was performed in thiosulfate minimal medium pH 7.0 (1 g/L KNO₃, 1 g/L KH₂PO₄, 0.5 g/L NH₄Cl, 15 g/L Na₂S₂O₃ and 60 g/L NaCl pH 7.0) at 30° C. for 48 h with 180 rpm agitation. The initial cultivation of H. neapolitanus was performed in ATCC 290 medium for Thiobacilli (1.2 g/L Na₂HPO₄, 1.8 g/L KH₂PO₄, 0.1 g/L MgSO₄×7H₂O, 0.1 g/L (NH₄)₂SO₄, 0.03 g/L CaCl₂), 0.02 g/L FeCl₃ and 0.02 g/L MnSO₄) supplemented with thiosulfate (10 g/L) at 30° C. for 24 h with 180 rpm agitation.

### New Isolate Identification and Genome Sequencing

Classification of each brine spring isolate was performed by 16S rDNA analysis of individual colonies. PCR was performed using primers specific for the 16S genomic hypervariable region, (aggagatataccatgCCTACGGGNGGCWGCAG—SEQ ID NO:34) and (tggtggtgctcgagGACTACGGGTATCTAATCC—SEQ ID NO:35), which contained 5′ overhangs (lower case) to allow PCR products to be cloned into pET-28b(+) vector. The latter was performed using In-Fusion cloning (Takara Bio) with pET-28b linearised by PCR (primers: CTCGAGCACCACCACCACC (SEQ ID NO:36) and CTCTAGAAATAATTTTGTTTAACTTTAAGAAGG-AGATATACCatg (SEQ ID NO: 37)). Partial 16S rDNA sequences were determined by plasmid sequencing, and species identity was inferred by homology using the EMBL phylogeny tools (https://www.ebi.ac.uk/) (Janda and Abbott, 2007. J. Clin. Microbiol. 45, 2761-2764). One isolate (15) was identified as a Halomonas species and named Halomonas rowanensis.

A partial genome sequencing of isolate H. rowanensis was performed by MicrobesNG (Birmingham, UK) using DNA isolated from cultures grown on LB60 agar plates. Subsequent high coverage full genome sequence data for this strain was obtained by in house Pacbio Sequel next generation sequencing using DNA purified from overnight cultures in LB60 using a Monarch genomic DNA isolation kit (New England Biolabs). In this process, genomic DNA was sheared to generate approximately 10 kb fragments using g-TUBES (Covaris) following the manufacturer's instructions. DNA quality was verified using a Fragment Analyzer using the DNF-930 protocol (Agilent). The Express Template Prep Kit 2.0 procedure was used to process the samples for sequencing with multiplexing using the barcoded overhang adapter kit (Pacific Biosciences). Data was acquired using the Pacific Biosciences Sequel system. Following demultiplexing the genomic sequences were assembled using the Resequencing algorithm of SMRT Link 8.0. This yielded mean coverage depth of 280 of two assembled contigs 3,812,367,3 and 229,491 bp, with around 64% of the annotated proteins identified and ~36% hypothetical Annotated files were inspected for quality of annotation and the presence of known biosynthetic pathways using Artemis (Carver et al., 2012) and Patrik (Davis et al., 2020. Nucleic Acids. Res. 48, D606-D612).

### Phylogenetic Analysis

16S rDNA sequences of the isolates were aligned using MUSCLE (v. 3.7), trimmed using TrimAl (v. 1.3) and used to generate a distance matrix with DnaDist (Phylip v. 3.68) (Felsenstein, 1989. Phylogeny Inference Package (Version 3.2). Cladistics 5, 164-166), via the Phylemon2 web portal (http://phylemon.bioinfo.cipf.es/index.html) (Madeira et al., 2019). Phylogenetic trees in Newick format were generated using the Fitch-Margoliash method via WebPhylip (Lim and Zhang, 1999) and trees visualised with ETE Toolkit TreeView (Huerta-Cepas et al., 2016. Mol. Biol. Evol. 33, 1635-1638).

### Metabolic Pathway Mapping

Annotated Pacbio genome sequences were used to build a new database within the PathoLogic tool of the BioCyc Pathway Tools (v 23.0; P10) (Karp et al., 2021. Brief Bioinform. 22, 109-126). Default settings were used during the database initialisation, replicon specification and build to identify metabolic pathways from the genome data. These pathways were then used to inform manual homology searches and improve genome annotation through NCBI BLASTp (https://blast.ncbi.nlm.nih.gov/Blast.cgi) with varied parameters decided based on the similarity of the sequence in question with those in the database.

### Structure Prediction and Structure Homology Searches

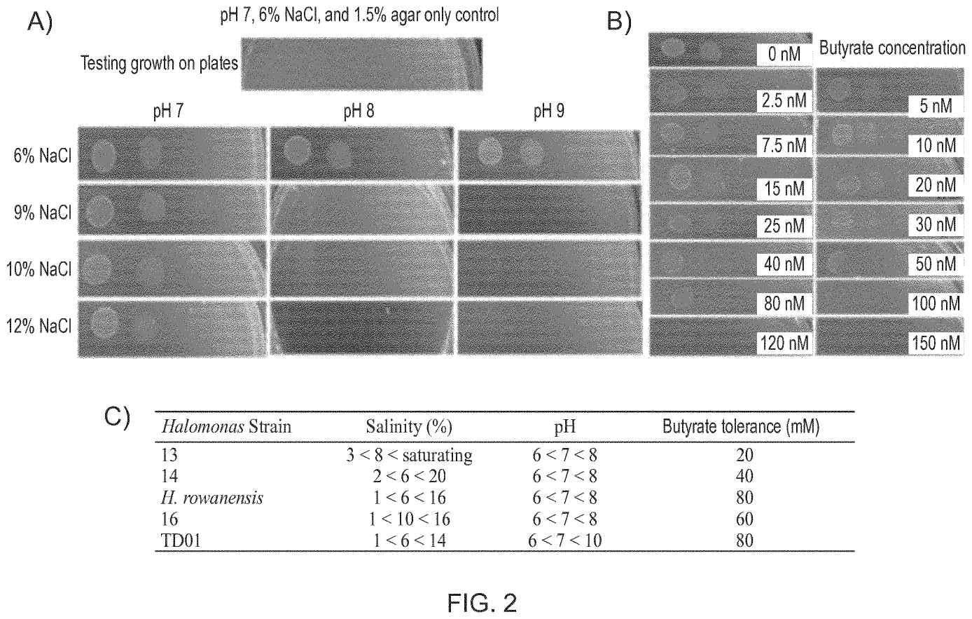

Validation of the annotation of putative carbon fixation and thiosulfate utilisation pathway genes in the genome sequence of H. rowanensis (FIG. 2) was performed by generating three dimensional models using the Alphafold 2.0 server (Jumper et al., 2021. Nature 596, 583-589). This was performed using the recommended default parameters for prokaryotic peptide sequences. These structures were viewed and interrogated in Pymol (Schrödinger and DeLano, 2020. PyMOL. Retrieved from http://www.pymol.org/pymol). The DALI webserver (Holm, 2020. Protein Sci. 29, 128-140) was used to match the predicted structures to the entire PDB database by homology with the recommended default settings and the resulting match with the lowest RMSD alignment was viewed and manual alignments performed in Pymol. The resulting alignment was interrogated for comparison of key residues from the predictions in the active site or those interacting with bound ligands in the published PDB structures.

### Bacterial Milking for Ectoine Recovery

Ectoine was harvested from Halomonas cultures using a modified ‘bacterial milking’ method described previously (Sauer and Galinski, 1998. Biotechnol. Bioeng. 59, 128-128). Cultures (50 mL) were harvested by centrifugation at 4000 g for 5 min at room temperature. The pellet was resuspended in 5 mL of ultrapure water (18.2 MΩ·cm) and vortexed for 1 min until homogeneous. The suspension was incubated for 10 min at room temperature and centrifuged as before. The milky white supernatant was retained and the ectoine content was quantified by HPLC using an Agilent 1260 Infinity HPLC with a 1260 ALS autosampler, TCC SL column heater, a 1260 refractive index detector (RID). Samples were run on an Agilent Hi-Plex-H column (300×7.7 mm) using 2.5 mM sulfuric acid as the mobile phase (0.6 mL/min) at 60° C. for 30 min. Ectoine quantitation (retention time of 6.3 min) was performed by comparing peak areas to a standard curve generated from authentic standards.

### Radiometric Bicarbonate Uptake and Fixation

Halomonas species were grown in LB60 or the thiosulfate medium (Mishra et al., 2017), while ATCC290-medium for thiobacilli was used for H. neapolitanus. Both E. coli and S. enterica were grown in LB. Cell cultures were grown to exponential phase, harvested by centrifugation, and were resuspended in Rubisco assay buffer (100 mM EPPS, pH 8.0, and 20 mM MgCl₂). The ¹⁴C (H¹⁴CO₃⁻) fixation assay was performed as described previously (Sun et al., 2016. Plant Physiol. 171, 530-541).

Cell cultures were grown to exponential phase using the medium above, harvested by centrifugation and resuspended in filtered nitrogen-bubbled culture medium to OD 600 nm of 4. The whole cell ¹⁴C uptake assay was performed according to previous studies (Sun et al., 2016. Plant Physiol. 171, 530-541) with several modifications. Radiolabelled NaH¹⁴CO₃ (10 mM final concentration) was added, and the cultures were incubated at 37° C. for 10, 15 and 60 minutes. Cell permeabilization was performed by the addition of alkyltrimethylammonium bromide (MTA) to a final concentration of 0.03% (w/v). Assay termination was performed by adding formic acid (10% (v/v)) and boiling the mixtures on heating blocks (98° C.) until dry, removing any unconverted NaHCO₃ as CO₂ gas. The powders were resuspended in water (100 μl), mixed with scintillation cocktails (1 mL; Ultima Gold XR, PerkinElmer) and counted for radioactivity (Tri-Carb, PerkinElmer). Three biological repeats were performed for each sample and the data were normalised to the cell density (OD600 nm).

### Example 2—Results and Discussion

### Isolates Identification and Genome Sequencing

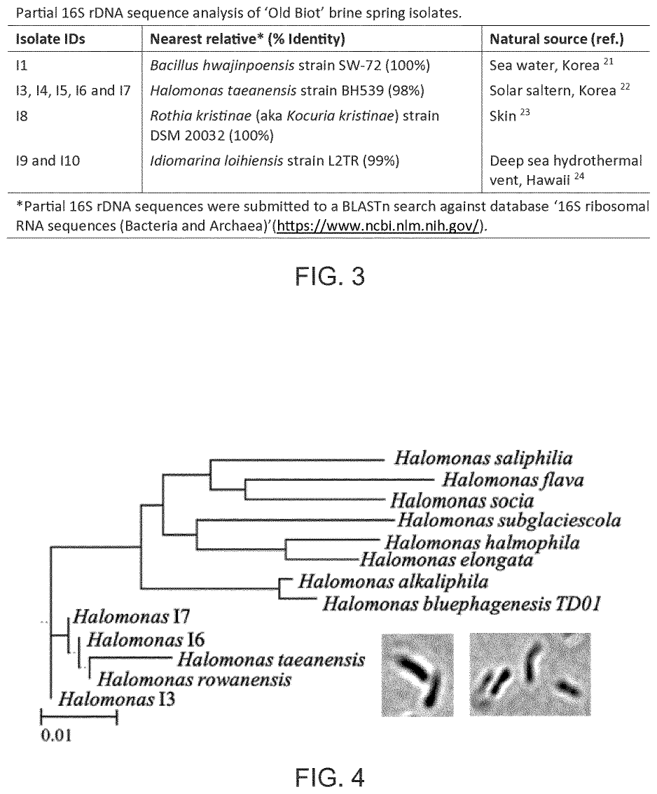

A variety of native halophilic or halotolerant bacteria were isolated from a natural brine spring (pH 7.1) by enriching with culture medium specific for Halomonas (Tan et al., 2011. Bioresour. Technol. 102, 8130-8136), but at neutral pH. Partial 16S rDNA, sequence analysis of individual isolates revealed the presence of some Gram-positive Bacillus species (orange-coloured colonies) and a Kocuria sp. strain (small, white colonies). In addition, Gram-negative bacteria were present, including two potential Idomarina species (19-110) and several Halomonas species (isolates 13-17) (FIG. 3).

Partial genome sequences were obtained for Halomonas 14 and Idiomarina 19 isolates, with a more complete genome sequence generated for the fastest growing Halomonas strain 15 (66.5% annotated). This compares to the 89% annotation of the H. bluephagenesis TD01 strain. The 16S rDNA genes for Halomonas st. 14 and 15 were identical, with a 99% identity to the known organism Halomonas taeanensis strain BH539 (Lee et al., 2005. Int. J. Syst. Evol. Microbiol. 55, 2027-2032) (FIG. 4). This strain was more distinct from the robust industrial strain H. bluephagenesis TD01, which prefers a highly alkaline environment. The 15 (and by homology 14) strain was designated as Halomonas rowanensis. For the two Idiomarina isolates, the 16S rDNA sequences differed in 2 base positions, suggesting two different species or strains were present.

The genome sequence for H. rowanensis revealed it contained all the expected genes required to support a heterotrophic lifestyle. This included complete pathways for glycolysis, TCA cycle with glyoxylate shunt, pentose phosphate and Entner-Duodoroff pathways. Genes for a fully functional aerobic electron transport chain were also present, such as cytochrome c oxidase for utilising oxygen as terminal electron acceptor and a proton translocating ATP synthase for chemiosmotic energy generation. The potential use of nitrate as an alternative terminal electron acceptor was also inferred by genes for respiratory nitrate reductase as well as genes permitting electron transfer from donor compounds formate and glycerol-3-phosphate.

### Halomonas Isolates Characterisation

The Halomonas isolates are small aerobic non motile rods (FIG. 4 insets), that grow heterotrophically on amino acid-based carbon sources. Each Halomonas isolate was screened for its tolerance towards salinity (NaCl) and optimal pH. The isolates were found to tolerate higher NaCl concentrations than the control H. bluephagenesis TD01 strain (FIG. 2), but preferred neutral pH at higher salinities (9-12% NaCl). This is consistent with the naturally high salinity (~20%) and neutral pH observed for the native spring they were isolated from.

Given our ultimate aim of biological propane production, the Halomonas isolates were tested for tolerance towards the precursor butyric acid (Amer et al., 2020b. Energy Environ. Sci. 13, 1818-1831). The highest butyric acid tolerance was found with H. rowanensis (80 mM), similar to engineered H. bluephagenesis TD01 (Amer et al., 2020a. Biotechnol. Biofuels 13, 125.) (FIG. 2).

Salt Tolerance in H. rowanensis

The mechanism(s) of salt tolerance of the new Halomonas species was investigated by genome mining for pathways known to be involved in osmoregulation. Genes associated with salt tolerance in Halomonas biemenensis include the sodium-translocating NADH: quinone oxidoreductase (nqrA) and an NAD-specific glutamate dehydrogenase (gdhB). These genes are associated with sodium efflux and the production of the secondary compatible solute glutamate (Chen et al., 2017. Sci. Rep. 7, 13037.). Both genes were found to be present in the genomes of both the Halomonas and Idiomarina isolates 14, 15, 19 and 110 (results not shown).

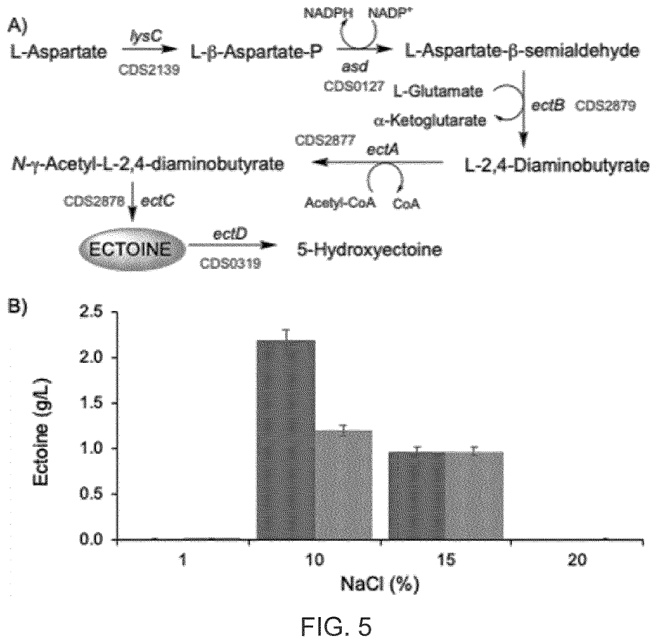

Halomonas species are known to counteract osmotic pressure by the production and intracellular accumulation of significant levels of the compatible solute ectoine (Nakayama et al., 2000. Plant Physiol. 122, 1239-1247). This compound is valuable product within biotechnology and cosmetics industries and has many medicinal uses. For example, ectoine is used as biofunctional stabilizers, skin protectors and potential drugs for diseases, such as Alzheimer's and rhinoconjunctivitis (Liu et al., 2021. Microb. Cell Fact. 20, 76.). Annotation of the H. rowanensis genome revealed the presence of a likely ectoine biosynthesis pathway (FIG. 5a). This included the ectABC operon and genes for aspartate kinase (lysC) and aspartate semialdehyde dehydrogenase 1 (asd1) (Liu et al., 2021. Microb. Cell Fact. 20, 76). In addition, the gene for ectoine dioxygenase (ectD) was present, which produces hydroxyectoine from ectoine. The osmoregulated solute TRAP transporter (teaABC) was also identified, which mediates the uptake of ectoine and hydroxyectoine in Halomonas elongata (Grammann et al., 2002. J. Bacteriol. 184, 3078-3085).

Ectoine can be obtained naturally from Halomonas elongata by a ‘bacterial milking’ process (Sauer and Galinski, 1998. Biotechnol. Bioeng. 59, 128-128). Bacterial milking was performed on H. rowanensis and a control H. bluephagenesis TD01 strain, the latter also known to generate large quantities of ectoine (Tao et al., 2017. Microb. Cell Fact., 1-11). As expected, high quantities of ectoine were produced by both Halomonas strains under high salt conditions (10-15% NaCl; FIG. 5b). Both strains generated similar titres of ectoine at 15% salinity (0.97 g/L), comparable to titres achieved by H. elongata (Sauer and Galinski, 1998. Biotechnol. Bioeng. 59, 128-128). However, H. bluephagenesis TD01 generated nearly double the ectoine levels than H. rowanensis at only 10% NaCl (2.19 vs 1.20 g/L, respectively). Therefore, H. rowanensis has a potential as a new industrial chassis for the non-sterile production of ectoine.

Chemoautotrophic Growth of H. rowanensis from CO₂ in Wastewater

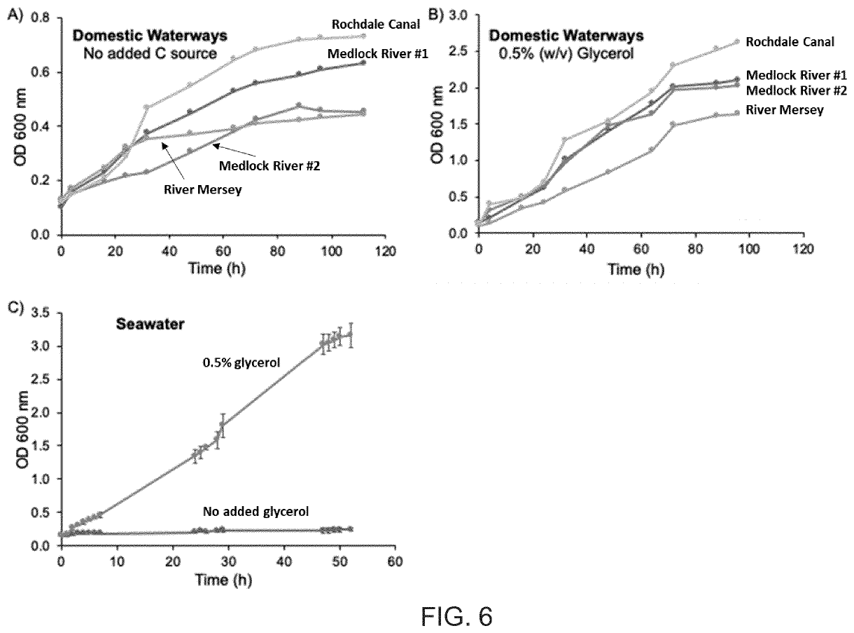

A key advantage of using Halomonas as an industrial microbial chassis is its ability to grow under non-sterile conditions in sea water and waste water (Amer et al., 2020b. Energy Environ. Sci. 13, 1818-1831). The growth of H. rowanensis was tested on salinity adjusted seawater (Irish sea) and polluted river or canal water collected from waterways around the Greater Manchester region (Medupin et al., 2020. https://doi.org/10.3390/w12030848). In each case, H. rowanensis grew well when wastewater was supplemented with glycerol as a carbon source (FIG. 6). Surprisingly, significant growth of H. rowanensis was detected in domestic waterway samples, but not seawater, in the absence of exogenous carbon sources. This suggested either significant carbon sources were naturally present in the Manchester waterways or H. rowanensis is capable of chemoautotrophic growth on inorganic carbon. The latter is not without precedent, as the thermotolerant Halomonas stevensii is known to fix CO₂ using thiosulfate as a sole energy source (Mishra et al., 2017. Process Biochem. 55, 133-145).

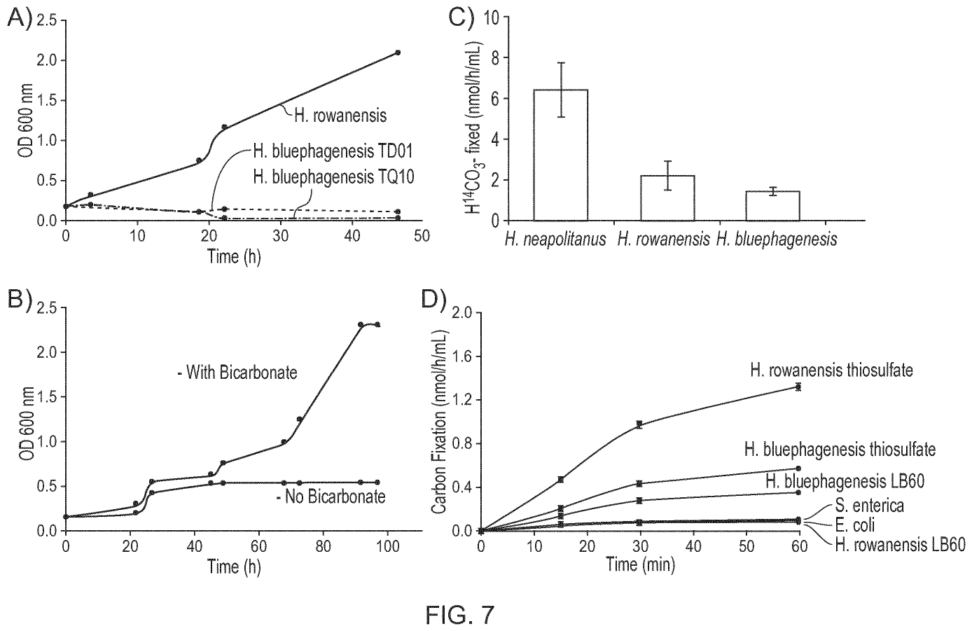

Both H. rowanensis and the H. bluephagenesis strains TD01 and TQ10 were investigated to determine if they are capable of chemoautotrophic or mixotrophic growth phenotypes using thiosulfate as the sole energy source (Mishra et al., 2017. Process Biochem. 55, 133-145). After repeated subculturing into organic carbon-free thiosulfate medium, only H. rowanensis was capable of significant growth (FIG. 7a). The addition of NaHCO₃ to the medium resulted in an enhancement in overall biomass production (FIG. 7b), suggesting H. rowanensis can fix CO₂ into organic carbon (Faulkner et al., 2017. Nanoscale 9, 10662-10673; Sun et al., 2016. Plant Physiol. 171, 530-541), similar to the growth of H. stevensii under chemoautotrophic conditions (Mishra et al., 2017. Process Biochem. 55, 133-145). Given that H. rowanensis also grows efficiently under heterotrophic conditions, this suggests this organism may be a facultative chemoautotroph.

To assess the potential of CO₂ fixation by H. rowanensis, radiolabelling experiments were performed with ¹H¹⁴CO3− and looked for label incorporation into cellular biomass. The NaH¹⁴CO3 was fed externally during growth, so label incorporation is a measure of both the efficiency of bicarbonate uptake systems as well as carbon fixation and retention intracellularly. Radiolabelled bicarbonate incorporation was detected for H. rowanensis (2.21±0.75 nmol/h/mL at OD 600 nm=4.0; FIG. 7c), which is equivalent to 2.47+0.84 nmol/h/g cells. This is approximately 3-fold lower than label incorporation under the same growth conditions by a known chemoautotroph H. neapolitanus (6.40±1.36 nmol/h/mL). Interestingly, H. bluephagenesis TD01 also displayed ¹⁴C incorporation (1.42±0.20 nmol/h/mL), despite a lack of growth in thiosulfate medium. This suggests it contains the machinery for carbon fixation, but not via thiosulfate as an energy source.

Further radiolabelled bicarbonate incorporation studies were performed with H. rowanensis and H. bluephagenesis TD01 under heterotrophic (LB60) and chemoautotrophic (thiosulfate) growth conditions. In this case, cells were permeabilised to remove the dependence on bicarbonate uptake into the cells. The highest levels of ¹⁴C incorporation were seen with H. rowanensis cultivated under chemoautotrophic conditions (1.32±0.03 nmol/mL), with a near 4-fold reduction when grown initially in heterotrophic growth medium (0.34±0.01 nmol/mL; FIG. 7d). This suggests a full carbon fixation pathway may be upregulated when the culture is transitioned into autotrophic growth medium, rather than the organism simply containing a few genes enabling it to survive brief periods of fixed carbon starvation.

Radiolabelled bicarbonate incorporation was apparent with H. bluephagenesis TD01 during the 1 h assay despite its lack of growth in thiosulfate-base chemoautotrophic medium. The incorporation was less efficient that H. rowanensis (0.57±0.01 nmol/mL; FIG. 7d), but also showed the upregulation when cells were transferred into chemoautotrophic medium. This suggests that carbon fixation may be a common trait within the Halomonas genus.

### Putative Carbon Fixation Pathway

Classic chemoautotrophic bacteria, such as H. neapolitanus, fix CO₂ via the Calvin-Bensen-Bassham cycle, with energy supplied by sulfur oxidation pathways (Dou et al., 2008. J. Biol. Chem. 283, 10377-10384). A carbon-concentration mechanism is present in H. neapolitanus via the presence of bicarbonate transporters, carbonic anhydrase and α-carboxysomes, which enclose the key carbon fixation enzyme ribulose 1,5-bisphosphate carboxylase/oxygenase (RubisCO) (Badger and Price, 2003. J. Exp. Bot. 54, 609-622). However, annotation of the H. rowanensis genome did not reveal the presence of any gene involved in the Calvin-Bensen-Bassham cycle or evidence of α-carboxysome formation. The quinoprotein dehydrogenase-associated SoxYZ-like carrier gene was found in the genome of Halomonas TD1.0, but no further genes involved in α-carboxysome formation were annotated.

The initial annotation of H. rowanensis contained a significant quantity of non-annotated proteins (66.5% complete), due to the low sequence homology to database genes. However, further annotations of the genome were performed from sequence homology using BLAST paired with RAST annotation, with confirmation of the overall enzyme class and likely presence of the appropriate active site residues/arrangement via AlphaFold structural modelling (Jumper et al., 2021. Nature 596, 583-589). The new annotated genome identified a putative complete reductive tricarboxylic acid (rTCA) cycle in H. rowanensis (FIG. 8) (Nunoura et al., 2018. Science 359, 559-563). This pathway has been found in other chemoautotrophs, such as Chlorobium, Desulfobacter hydrogenophilus and some members of the thermophilic Aquificales order and archaeal Thermoproteaceae family (Campbell and Cary, 2004. Appl. Environ. Microbiol. 70, 6282-6289).

The rTCA cycle generates one molecule of oxaloacetate from four molecules of CO₂ and requires 4-5 mol of adenosine 5′-triphosphate (ATP) (Campbell and Cary, 2004. Appl. Environ. Microbiol. 70, 6282-6289). Essential genes for this pathway are required to overcome key energetically unfavourable reverse reaction steps. This includes ATP citrate lyase, which catalyzes the cleavage of citrate into acetyl-CoA and oxaloacetate in a CoA- and ATP-dependent manner (Step 11 of FIG. 8). Other key enzymes are two of the four carbon dioxide-fixing enzymes 2-oxoglutarate: ferredoxin oxidoreductase (Step 7 of FIG. 8), and pyruvate: ferredoxin oxidoreductase (Campbell and Cary, 2004. Appl. Environ. Microbiol. 70, 6282-6289).

At least one annotated gene was identified for each step for the rTCA cycle, whose likely function were predicted by AlphaFold structural simulation. Other genes identified included enzymes catalysing the interconversion of malate to glyoxylate and acetyl CoA or pyruvate and those involved in the hydroxybutyrate (Huber et al., 2008. Proc. Natl. Acad. Sci. U.S.A 105, 7851-7856) and hydroxypropionate (Hügler and Fuchs, 2005. Methods Enzymol. 397, 212-221) cycles. A gene for γ-butyrobetaine hydroxylase was identified, which could potentially act as an alternative enzyme to catalyse steps 6-7 (succinate to α-ketoglutarate) in the presence of ascorbate (Leung et al., 2010. Chem. Biol. 17, 1316-1324). Given the presence of key enzymes citrate lyase, NADPH dependent malic enzyme and malate dehydrogenase, the interconversion of citrate and pyruvate is possible.

Putative Thiosulphate Utilisation Pathways in H. rowanensis

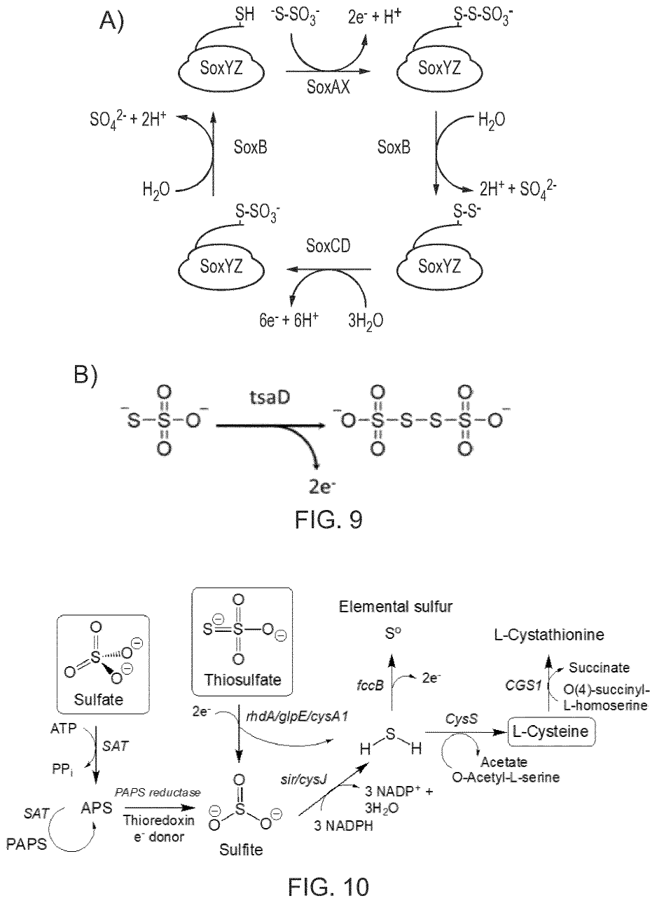

Chemolithoautrophic bacterium obtain energy to fix atmospheric CO₂ typically by extracting electrons from reduced ferrous (Fe[II]), thiosulfate (S₂O₃⁻²) or ammonium (NH₄⁺) ions (Amend et al., 2003. Geobiology 1, 37-58). For example, energy generation in H. neapolitanus and Paracoccus pantrophus proceeds via the oxidation of thiosulfate to sulfate using the Sox system (Veith et al., 2012. J. Bacteriol. 194, 677-685; Whaley-Martin et al., 2019. Front. Microbiol. 10), delivering eight electrons to the quinone pool for ATP generation (FIG. 9a). Alternatively, some chemoorganoheterotrophic bacteria, including Halomonas species, are known to derive energy from the oxidation of thiosulfate to tetrathionate (FIG. 9b) via the action of thiosulfate dehydrogenase (tsaD) (Kurth et al., 2016. J. Biol. Chem. 291, 24804-24818).

Annotation of the genomes of H. rowanensis and Halomonas strain 14 revealed the presence of a variety of genes involved in the transport of thiosulfate to sulfite, sulfide and elemental sulfur (Supplementary Table S3). However, there were no Sox genes system or tsaD annotated in either genome that could explain how H. rowanensis could derive energy to fix CO₂ in chemoautotrophic thiosulfate medium. Given the relative incompleteness of the H. rowanensis genome compared to H. bluephagenesis TD01, it is likely that the genes involved in chemoautotrophic energy generation are buried within the putative proteins. Therefore, further work is required before the mechanism(s) of energy generation is determined for H. rowanensis and H. bluephagenesis TD01.

The presence of multiple thiosulfate transferases and sulfate adenyltransferases suggest H. rowanensis contains the assimilatory and dissimilatory sulfate reduction and oxidation pathways. The sulfur metabolism genes annotated in H. rowanensis suggests they play a role as a sulphur source for L-cysteine and related compounds (FIG. 10). For example, several putative thiosulfate sulfurtransferases (e.g. rhdA and glpE) could reduce thiosulfate to sulfite (Ray et al., 2000. J. Bacteriol. 182, 2277-2284). Further NADPH-dependent reductions could lead to the production of sulfides, which can act as a sulfur donor to O-acetyl-L-serine to generate L-cysteine (Agren et al., 2008. J. Biol. Chem. 283, 31567-31574). Genes for the conversion of sulfate to sulfite via adenosine-5′-phosphosulfate (APS) are also present (FIG. 10). As these pathways overall are net energy requiring, they are unlikely to be coupled directly to CO₂ fixation.

Chemoautotrophic Propane Production from CO₂

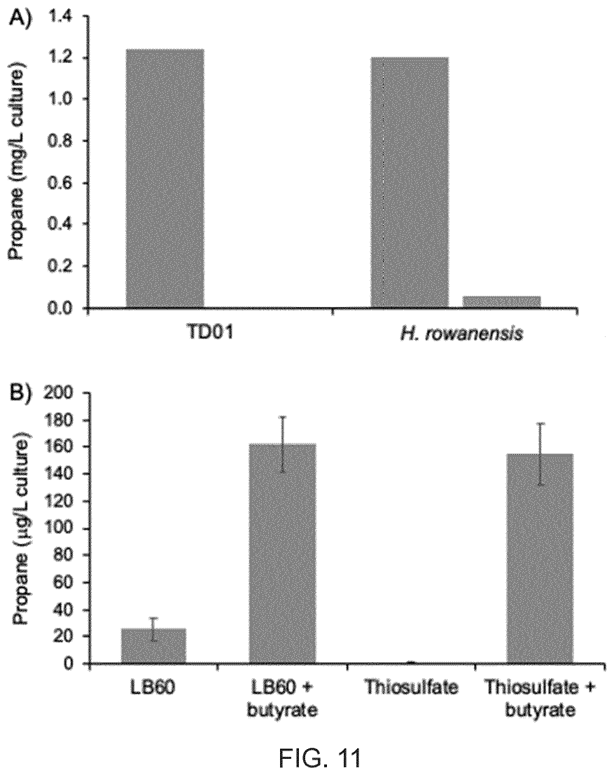

The heterotrophic biopropane/bio-LPG production in H. bluephagenesis TQ10 cultivated on biodiesel waste glycerin and amino acids has been previously demonstrated (Amer et al., 2020a. Biotechnol. Biofuels 13, 125; Amer et al., 2020b. Energy Environ. Sci. 13, 1818-1831; Trisrivirat et al., 2020. Synth. Biol. 5). This was based on the expression of recombinant fatty acid photodecarboxylase variant G462V from Chlorella variabilis NC64A (CvFAP_{G462V}), which catalyses the decarboxylation of butyric acid to propane (Sorigué et al., 2017. Science 357, 903-907). H. rowanensis was tested for propane production by expressing CvFAPG_{462V} with a weak (P7) or strong (P102) constitutive promoter and cultivating under blue light. Propane production from CO₂ was more significant when using the weaker P7 promoter (61.8 μg/L culture; FIG. 11a). Supplementation with butyric acid (substrate and carbon source) led to a 19-fold increase in propane titres (1.2 mg/L culture). The relatively low titres compared to prior studies with H. bluephagenesis TQ10 under heterotrophic conditions reflects the slower growth rate, low butyrate concentration and relatively high apparent kinetic constant of CvFAP (Amer et al., 2020b. Energy Environ. Sci. 13, 1818-1831).

Surprisingly, switching to a higher strength promoter system yielded lower overall yields of propane under heterotrophic and chemoautotrophic growth conditions (FIG. 11b). This suggests the high expression of CvFAP_{G462V} may impart some toxicity on the system. Overall, propane titres of H. rowanensis from CO₂ (thiosulphate medium without butyric acid) were around 10-fold lower than photoautotrophic propane production by Synechcocystis PCC 6803 (~11.1±2.4 mg propane/L/day) (Amer et al., 2020b. Energy Environ. Sci. 13, 1818-1831). However, the latter strain had been bioengineered to accumulate small chain fatty acids by knocking out the native fatty acyl ACP synthase gene (Aaas) and to co-express CvFAP_{G462V} with a butyryl-ACP thioesterase from Bacteroides fragilis (Tes4) (Zhang et al., 2016. Biotechnol. Biofuels. 9, 80).

Supplementing cultures with butyric acid is likely to switch the metabolism towards heterotrophic growth as Halomonas species are known to grow on butyric acid as a carbon source (Amer et al., 2020b. Energy Environ. Sci. 13, 1818-1831). The high ectoine titres by H. rowanensis show that there is potential for use of this organism as a robust microbial chassis when it is engineered to produce suitable high value and high titre secondary products.

### Example 3—Conclusions

The recruitment of environmental isolates as de novo microbial chassis is not a new concept. The practice of screening for microorganisms of a specific genera or growth condition can sometimes lead to surprising growth phenotypes, which can open up alternative approaches to achieve the desired bioproduction goal. The discovery of a facultative chemoautotrophic Halomonas species with relatively rapid growth on inorganic carbon sources raises the possibility of dramatically reducing the carbon footprint of chemicals and fuels bioproduction by utilising industrial waste gases as the carbon (CO₂) and energy (sulphur compounds) sources.

Thiosulfate and inorganic sulphur contaminated wastewater can be found in the mining industry (Natarajan, 2018. Biotechnology of Metals, 179-210), steelworks (Vachon and Schmidtke, 1981. Steel Industry Wastes. Journal (Water Pollution Control Federation) 53, 844-847), offshore gas, shale oil (Stuber et al., 1978. J. Environ. Sci. Health, Part A: Environ. Sci. Eng. 13, 663-675) and water dichlorination processes (Oh et al., 2008. Environ. Toxicol. 23, 211-217). A potential scenario could be the capture of both industrial CO₂ from the flue gas and thiosulfate-contaminated water found at steel mills as the basis for a bioproduction medium. This would effectively be a means to bioremediate thiosulfate whilst capturing CO₂ emissions as both biomass and propane.

There are considerable cost savings to be achieved by exploiting the ability of halophiles to be cultivated under non-sterile growth conditions without the need for fresh water. The relative ease at which the Halomonas genetic toolbox was transmittable to an unmodified natural isolate highlights the potential of environmental screening to discover more unique potential microbial chassis. Overall, the utilisation of a chemoautotrophic halophilic industrial microbial chassis could improve the sustainability of the process in addition to an overall reduction in the carbon footprint. The generation of valuable biochemicals and fuels from CO₂ could increase the energy security and could contribute towards global carbon management targets and help achieve clean air directives.

### NUMBERED PARAGRAPHS

1. Halomonas rowanensis deposited with European Collection of Authenticated Cell Cultures on 10 Feb. 2023 under accession number 23021001, or a derivative of the deposited Halomonas rowanensis.

2. The derivative of Halomonas rowanensis according to paragraph 1, wherein the derivative expresses one or more heterologous genes for the production of a carbon compound.

3. The derivative of Halomonas rowanensis according to paragraph 1 or paragraph 2, wherein the derivative expresses a heterologous fatty acid photodecarboxylase.

4. The derivative of Halomonas rowanensis according to paragraph 3, wherein the fatty acid photodecarboxylase comprises an amino acid sequence having at least 70% sequence identity to SEQ ID NO: 1.

5. The derivative of Halomonas rowanensis according to any previous paragraph, wherein the derivative expresses a pHALP7-CvFAPG_{462V} or pHALP102-CvFAPG_{462V} plasmid.

6. A microbial culture comprising the Halomonas rowanensis or the derivative of Halomonas rowanensis according to any previous paragraph.

7. A method, the method comprising culturing the Halomonas rowanensis or the derivative of Halomonas rowanensis according to any of paragraphs 1 to 5.

8. The method of paragraph 7, wherein the Halomonas rowanensis or the derivative of Halomonas rowanensis is cultured in the presence of carbon dioxide.

9. A method of producing a carbon compound, the method comprising culturing the Halomonas rowanensis or the derivative of Halomonas rowanensis according to the method of paragraph 7 or paragraph 8, wherein the method further comprises isolating a carbon compound from the culture.

10. A method of producing a carbon compound, the method comprising culturing a Halomonas cell in the presence of carbon dioxide, wherein the Halomonas cell is capable of fixing carbon dioxide, the method further comprising isolating a carbon compound from the culture.

11. The method of producing a carbon compound according to paragraph 10, wherein the Halomonas cell is a chemoautotroph or a facultative chemoautotroph.

12. The method of producing a carbon compound according to any one of paragraphs 9 to 11, wherein the Halomonas cell is cultured in the presence of a sulfur containing compound.

13. The method of producing a carbon compound according to paragraph 12, wherein the sulfur containing compound is a thiosulfate, sulfuric acid, sulfur, a sulfate, and/or a sulfite.

14. The method of producing a carbon compound according to any one of paragraphs 9 to 13, wherein the carbon compound is a C2-C17 carbon compound.

15. The method of producing a carbon compound according to any one of paragraphs 9 to 14, wherein the carbon compound is an alkane.

16. The method of producing a carbon compound according to paragraph 15, wherein the carbon compound is a C₃-C6 alkane.

17. The method of producing a carbon compound according to paragraph 16, wherein the C3-C6 alkane is propane, butane, and/or isobutane.

18. The method of producing a carbon compound according to any one of paragraphs 9 to 17, wherein the Halomonas expresses one or more heterologous genes for the production of a carbon compound.

19. The method of producing a carbon compound according to any one of paragraphs 9 to 17, wherein the Halomonas expresses a heterologous fatty acid photodecarboxylase.

20. The method of producing a carbon compound according to paragraph 19, wherein the fatty acid photodecarboxylase comprises an amino acid sequence having at least 70% sequence identity to SEQ ID NO: 1.

21. The method of producing a carbon compound according to any one of paragraphs 9 to 18, wherein the Halomonas expresses a heterologous linalool synthase.

22. The method of producing a carbon compound according to paragraph 21, wherein the linalool synthase comprises an amino acid sequence having at least 70% sequence identity to SEQ ID NO:30.

23. The method of producing a carbon compound according to any one of paragraphs 9 to 18, wherein the Halomonas expresses a heterologous CAR and a heterologous TA.

24. The method of producing a carbon compound according to paragraph 23, wherein the CAR comprises an amino acid sequence having at least 70% sequence identity to SEQ ID NO: 43, 44, 45, 46, 47, or 48.

25. The method of producing a carbon compound according to paragraph 23, wherein the TA comprises an amino acid sequence having at least 70% sequence identity to SEQ ID NO:49, 50, 51, 52, or 53.

26. The method according to any one of paragraphs 9 to 25, wherein the culture is provided with carbon dioxide in addition to, or alternatively to, atmospheric carbon dioxide.

27. The method according to any one of paragraphs 9 to 26, wherein the Halomonas cell is cultured in an environment having a carbon dioxide concentration of at least 500 ppm.

28. The method of producing a carbon compound according to any one of paragraphs 9 to 27, wherein the Halomonas is cultured in a media comprising polluted water.

29. The method of paragraph 28, wherein the polluted water comprises carbon and/or sulfur containing compounds and the Halomonas cell reduces the concentration of the carbon and/or sulfur containing compounds in the polluted water.

### REFERENCES

A number of publications are cited above in order to more fully describe and disclose the invention and the state of the art to which the invention pertains. Full citations for these references are provided below. The entirety of each of these references is incorporated herein.
Amer, M., Hoeven, R., Kelly, P., Faulkner, M., Smith, M. H., Toogood, H. S., Scrutton, N. S. 2020a. Renewable and tuneable bio-LPG blends derived from amino acids. Biotechnol. Biofuels 13, 125. https://doi.org/10.1186/s13068-020-01766-0.
Amer, M., Wojcik, E. Z., Sun, C., Hoeven, R., Hughes, J. M. X., Faulkner, M., Yunus, I. S., Tait, S., Johannissen, L. O., Hardman, S. J. O., Heyes, D. J., Chen, G.-Q., Smith, M. H., Jones, P. R., Toogood, H. S., Scrutton, N. S. 2020b. Low carbon strategies for sustainable bio-alkane gas production and renewable energy. Energy Environ. Sci. 13, 1818-1831.https://doi.org/10.1039/D0EE00095G.
Chen, G.-Q., Jiang, X.-R. 2018. Next generation industrial biotechnology based on extremophilic bacteria. Curr. Opin. Biotechnol. 50, 94-100.https://doi.org/10.1016/j.copbio.2017.11.016.
Chen, Y.-H., Lu, C.-W., Shyu, Y.-T., Lin, S.-S. 2017. Revealing the saline adaptation strategies of the halophilic bacterium Halomonas beimenensis through high-throughput omics and transposon mutagenesis approaches. Sci. Rep. 7, 13037.https://doi.org/10.1038/s41598-017-13450-9.
Fu, X.-Z., Tan, D., Aibaidula, G., Wu, Q., Chen, J.-C., Chen, G.-Q. 2014. Development of Halomonas TD01 as a host for open production of chemicals. Metab. Eng. 23, 78-91.https://doi.org/10.1016/j.ymben.2014.02.006.
Mishra, S., Raghuvanshi, S., Gupta, S., Raj, K. 2017. Application of novel thermo-tolerant haloalkalophilic bacterium Halomonas stevensii for bio mitigation of gaseous phase CO₂: Energy assessment and product evaluation studies. Process Biochem. 55, 133-145. https://doi.org/10.1016/j.procbio.2017.01.019.
Trisrivirat, D., Hughes, J. M. X., Hoeven, R., Faulkner, M., Toogood, H., Chaiyen, P., Scrutton, N. S. 2020. Promoter engineering for microbial bio-alkane gas production. Synth. Biol. 5, ysaa022.https://doi.org/10.1093/synbio/ysaa022.
Zhang, X., Lin, Y., Chen, G.-Q. 2018. Halophiles as chassis for bioproduction. Adv. Biosyst. 2, 1800088.https://doi.org/10.1002/adbi.201800088.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. Halomonas rowanensis deposited with European Collection of Authenticated Cell Cultures on 10 Feb. 2023 under accession number 23021001, or a derivative of the deposited Halomonas rowanensis.

2. The derivative of Halomonas rowanensis according to claim 1, wherein the derivative expresses one or more heterologous genes for the production of a carbon compound.

3. The derivative of Halomonas rowanensis according to claim 1 or claim 2, wherein the derivative expresses a heterologous fatty acid photodecarboxylase.

4. The derivative of Halomonas rowanensis according to claim 3, wherein the fatty acid photodecarboxylase comprises an amino acid sequence having at least 70% sequence identity to SEQ ID NO: 1.

5. The derivative of Halomonas rowanensis according to any previous claim, wherein the derivative expresses a pHALP7-CvFAP_{G462V} or pHALP102-CvFAP_{G462V} plasmid.

6. A microbial culture comprising the Halomonas rowanensis or the derivative of Halomonas rowanensis according to any previous claim.

7. A method, the method comprising culturing the Halomonas rowanensis or the derivative of Halomonas rowanensis according to any of claims 1 to 5.

8. The method of claim 7, wherein the Halomonas rowanensis or the derivative of Halomonas rowanensis is cultured in the presence of carbon dioxide.

9. A method of producing a carbon compound, the method comprising culturing the Halomonas rowanensis or the derivative of Halomonas rowanensis according to the method of claim 7 or claim 8, wherein the method further comprises isolating a carbon compound from the culture.

10. The method of producing a carbon compound according to claim 9, wherein the Halomonas rowanensis or the derivative of Halomonas rowanensis is cultured in the presence of a sulfur containing compound.

11. The method of producing a carbon compound according to claim 10, wherein the sulfur containing compound is a thiosulfate, sulfuric acid, sulfur, a sulfate, and/or a sulfite.

12. The method of producing a carbon compound according to any one of claims 9 to 11, wherein the carbon compound is a C2-C17 carbon compound.

13. The method of producing a carbon compound according to any one of claims 9 to 12, wherein the carbon compound is an alkane.

14. The method of producing a carbon compound according to claim 13, wherein the carbon compound is a C3-C6 alkane.

15. The method of producing a carbon compound according to claim 14, wherein the C3-C6 alkane is propane, butane, and/or isobutane.

16. The method of producing a carbon compound according to any one of claims 9 to 15, wherein the derivative of Halomonas rowanensis expresses one or more heterologous genes for the production of a carbon compound.

17. The method of producing a carbon compound according to any one of claims 9 to 16, wherein the derivative of Halomonas rowanensis expresses a heterologous linalool synthase.

18. The method of producing a carbon compound according to claim 17, wherein the linalool synthase comprises an amino acid sequence having at least 70% sequence identity to SEQ ID NO:30.

19. The method according to any one of claims 9 to 18, wherein the culture is provided with carbon dioxide in addition to, or alternatively to, atmospheric carbon dioxide.

20. The method according to any one of claims 9 to 19, wherein the Halomonas rowanensis or the derivative of Halomonas rowanensis is cultured in an environment having a carbon dioxide concentration of at least 500 ppm.

21. The method of producing a carbon compound according to any one of claims 9 to 20, wherein the Halomonas rowanensis or the derivative of Halomonas rowanensis is cultured in media comprising polluted water.

22. The method of claim 21, wherein the polluted water comprises carbon and/or sulfur containing compounds and the Halomonas rowanensis or the derivative of Halomonas rowanensis reduces the concentration of the carbon and/or sulfur containing compounds in the polluted water.


SEQUENCE LISTING (56 sequences)

SEQ ID NO: 1 - protein, 654 aa, Chlorella sp.
  source 1..654: note = Chlorella variabilis
MASITSRASARASCSQANTRAGRVALSGGALLRPARPARSFVPARKQQQGAVRRGGALSARASAVEDIRKVLSDSSSPVAGQKYDYILVGGGTAACVLANRLSADGSKRVLVLEAGPDNTSRDVKIPAAITRLFRSPLDWNLFSELQEQLAERQIYMARGRLLGGSSATNATLYHRGAAGDYDAWGVEGWSSEDVLSWFVQAETNADFGPGAYHGSGGPMRVENPRYTNKQLHTAFFKAAEEVGLTPNSDFNDWSHDHAGYGTFQVMQDKGTRADMYRQYLKPVLGRRNLQVLTGAAVTKVNIDQAAGKAQALGVEFSTDGPTGERLSAELAPGGEVIMCAGAVHTPFLLKHSGVGPSAELKEFGIPVVSNLAGVGQNLQDQPACLTAAPVKEKYDGIAISDHIYNEKGQIRKRAIASYLLGGRGGLTSTGCDRGAFVRTAGQALPDLQVRFVPGMALDPDGVSTYVRFAKFQSQGLKWPSGITMQLIACRPQSTGSVGLKSADPFAPPKLSPGYLTDKDGADLATLRKGIHWARDVARSSALSEYLDGELFPGSGVVSDDQIDEYIRRSIHSSNAITGTCKMGNAGDSSSVVDNQLRVHGVEGLRVVDASVVPKIPGGQTGAPVVMIAERAAALLTGKATIGASAAAPATVAA

SEQ ID NO: 2 - protein, 594 aa, synthetic construct
MASAVEDIRKVLSDSSSPVAGQKYDYILVGGGTAACVLANRLSADGSKRVLVLEAGPDNTSRDVKIPAAITRLFRSPLDWNLFSELQEQLAERQIYMARGRLLGGSSATNATLYHRGAAGDYDAWGVEGWSSEDVLSWFVQAETNADFGPGAYHGSGGPMRVENPRYTNKQLHTAFFKAAEEVGLTPNSDFNDWSHDHAGYGTFQVMQDKGTRADMYRQYLKPVLGRRNLQVLTGAAVTKVNIDQAAGKAQALGVEFSTDGPTGERLSAELAPGGEVIMCAGAVHTPFLLKHSGVGPSAELKEFGIPVVSNLAGVGQNLQDQPACLTAAPVKEKYDGIAISDHIYNEKGQIRKRAIASYLLGGRGGLTSTGCDRGAFVRTAGQALPDLQVRFVPGMALDPDGVSTYVRFAKFQSQGLKWPSGITMQLIACRPQSTGSVGLKSADPFAPPKLSPGYLTDKDGADLATLRKGIHWARDVARSSALSEYLDGELFPGSGVVSDDQIDEYIRRSIHSSNAITGTCKMGNAGDSSSVVDNQLRVHGVEGLRVVDASVVPKIPGGQTGAPVVMIAERAAALLTGKATIGASAAAPATVAA

SEQ ID NO: 3 - protein, 564 aa, unidentified
  source 1..564: note = Aureococcus anophagefferens
MGRTLVLKVATTSYDYIIAGGGTAGCVLANRLSEDPSKKVLVLEAGDRGPNSPLVKIPVAILKLFKSAYDWNFATRPSEAVADRSLYVCRGKGLGGSSLTNVMLYNRGSANDYDAWAAACGDDSWGAEEMLGYFKKAEDCLVPAHRANHYHGVGGPYASSHVPYTNEMSTAFVEAAVEDGGVRNGDFNDWSTSQVGFGRFAVSQRKGARVDAATAYLPRKVRRRANLDVVRGAALSGVTWNANKATGVEFAFGGVSGIACGGEVILSGGAVHSPQMLMLSGVGAKAQLEEFGIPVVADRPGVGKNLQDHPACLVSWRGSAKAQGKSHSTQLRIPGTTKTSPKALLQWLFLGRGPLASPGCDHGGFAKVGAGDGDCDVQFRFLATKSITPDGMSTISDSYEAAVDHPDGLTIQTIVARPKSRAGEVKLASRDPAAKPVIENAYLSDEADVMTMVKALQKARSIASRAPLSAYAGHEEFPGEDVADERQLAAYVRNTAHTANAVVGTCKMGESSDALAVVDNHLKVIGVSNLRVVDASVMPTLPGGQTAASTVALAEKAADLIKGG

SEQ ID NO: 4 - protein, 640 aa, Chondrus crispus
MASPCPAFATPIAVPRSTLTSLISSSSSCTPRPVRTPAPPTHRRLIHMAAPAGTVASTFRRTVPSSEAATTYDYIIVGGGAAGCVLANRLTEDPSTRVLLLEAGKPDDSFYLHVPLGFPYLLGSPNDWAFVTEPEPNLANRRLYFPRGKVLGGSHAISVMLYHRGHPADYTAWAESAPGWAPQDVLPYFLKSESQQSAVPNQDAHGYEGPLAVSDLARLNPMSKAFIKAAHNAAGLNHNPDFNDWATGQDGVGPFQVTQRDGSRESPATSYLRAAKGRRNLTVMTGAVVERILFENPAGSSTPVATAVSFIDSKGTRVRMSASREILLCGGVYATPQLLMLSGVGPAEHLRSHGIEIVADVPAVGQNLQDHAAAMVSFESQNPEKDKANSSVYYTERTGKNIGTLLNYVFRGKGPLTSPMCEAGGFAKTDPSMDACDLQLRFIPFVSEPDPYHSLADFATAGSYLQNRANRPTGFTIQSVAARPKSRGHVQLRSTDVRDSMSIHGNWISNDADLKTLVHGVKLCRTIGNDDSMKEFRGRELYPGGEKVSDADIEAYIRDTCHTANAMVGTCRMGIGEQAAVDPALQVKGVARLRVVDSSVMPTLPGGQSGAPTMMIAEKGADLIRAAARQADAATVGAAA

SEQ ID NO: 5 - protein, 627 aa, Chrysochromulina sp.
MMRRLVYICAVATVTAAISSRSVPTSARRLIALRGGVAAAEQLAEEPWDYIIVGGGAAGCVMAERLSAAEARVLVLEAGTDASRDLRIRVPAGLIKVFKSERDWDFTTEAGQGTSGRGIYLCRGKALGGSSCTNVMLYNRGSPADYNSWVAAGAEGWGPDSVLHYYRKSENYVGGASQYHGVDGPLSVSDVPYENELSTAFLRAAGELGYRRVHDFNDWSAPQEGFGRYKVTQRNGERCSAANAYLEGTEGRSNLCVRTGVHATRVTLEGSGDDLCAAGVEYIGADGKPSRAQLAQGGEVLLSAGAVQSPQLLMLSGIGPRAHLEEVGIEVRKELDNVGVGLADHPAVVVSCGSKKKVSVTDEIRLWGGSKTNPMALLRWLLWRRGPLTSVACEFGGFFKTKPDLKQADVQVRFVAARAMSPDGITTLQQLGAGAKFLSGYTTQIIACRPQSTGLVRLRSSDPLAQPMLQDVHLSDDADVATLREGIKLGRQLLAAKSFDQYRDEEVYPGVAVQSDEDIDAYVRKTTHSANALVGSCRMGRVDDQAAVLDPEMRVRGVGSLRVVDASAMPHIIGGQTCGPTIMMAEKAADLVLRQRAEINAYMQQAQAYLAASAGAATPALSPAQAA

SEQ ID NO: 6 - protein, 642 aa, unidentified
  source 1..642: note = Cyanidioschyzon merolae
MRSRYCFLLSSTPCKYAGQRSPFPASALAGVCAGGRLRNVTRNLRPGLRTLRASAETEHSQGTRQAQYDFIIVGAGAAGCVLANRLSTAQFSNGDRRYPRVLLLEAGDALAEAPYFEHIPLGFPQLIGSRLDYGFFSRENPTHLGGRGAVYLPRGRGEGGSHAISVMLVHRGSRHDYETWVKDYEALGWGPDDVLPYFKRLESNERTAQRGADGEAATALHGSDGPLRVSDQRSPNPLSLAFIEACLERGIRRNKDFNDWDHGQEGAGLFQVTQRDGRRESPATAYLQPVRSRRNLHIETNALAEHLVWSKDGRRVEGIRFIDRHGRRRAALAHCEVILAAGAINTPQLLMLSGLGPGAHLQDFGIPVVRDLPGVGQNLQDHAAVMLSYYAPDPYGKDRDKKRIFYTERLGKDPLVLAEYFLLGRGPLTSPVCEAGAFVHTQAVIGEPSCDLQLRFVPFFSDADPYKSLGEYRSGGHVLTNTSIRPAGFGLQAVAIRPRSRGRIELATIDPRARPIIHTGWLEDKRDLQTLLSGLKLGREILSGDSMRPYRGREAFPETLEDDLVTYIRRTCHTANAIVGTARMGTGRDAVVDPELRVHGVERLRVIDASVMPKIIGGQTGVPTMMIAERGADLVKKTWKLV

SEQ ID NO: 7 - protein, 611 aa, Chlamydomonas reinhardtii
MMLGPKTVTRGATKGAAPRSMAARRVGGARRLSVRAAAGPAGSEKFDYVLVGGGTASCVLANKLSADGNKKVLVLEAGPTGDAMEVAVPAGITRLFAHPVMDWGMSSLTQKQLVAREIYLARGRMLGGSSGSNATLYHRGSAADYDAWGLEGWSSKDVLDWFVKAECYADGPKPYHGTGGSMNTEQPRYENVLHDEFFKAAAATGLPANPDFNDWSHPQDGFGEFQVSQKKGQRADTYRTYLKPAMARGNLKVVIGARATKVNIEKGSSGARTTGVEYAMQQFGDRFTAELAPGGEVLMCSGAVHTPHLLMLSGVGPAATLKEHGIDVVSDLSGVGQNLQDHPAAVLAARAKPEFEKLSVTSEVYDDKCNIKLGAVAQYLFQRRGPLATTGCDHGAFVRTSSSLSQPDLQMRFVPGCALDPDGVKSYIVFGELKKQGRAWPGGITLQLLAIRAKSKGSIGLKAADPFINPAININYFSDPADLATLVNAVKMARKIAAQEPLKKYLQEETFPGERASSDKDLEEYIRRTVHSGNALVGTAAMGASPAAGAVVSSADLKVFGVEGLRVVDASVLPRIPGGQTGAATVMVAERAAALLRGQATIAPSRQPVAV

SEQ ID NO: 8 - protein, 617 aa, Coccomyxa sp.
  source 1..617: note = Coccomyxa subellipsoidea
MMASQSVFLGTRPATRSPLPIGRAGHGSAGRRALRVRAIIKSDNPAADKYDFILVGGGTAGCVLANRLTADGSKKVLLLEAGGANKAREVRTPAGLPRLFKSALDWNLYSSLQQAASDRSIYLARGKLLGGSSATNATLYHRGTAADYDAWGVPGWTSQDALRWFIQAENNCRGIEDGVHGTGGLMRVENPRYNNPLHEVFFQAAKQAGLPENDNFNNWGRSQAGYGEFQVTHSKGERADCFRMYLEPVMGRSNLTVLTGAKTLKIETEKSGGATVSRGVTFQVNGQDGSKHSAELAAGGEVVLCAGSIHSPQILQLSGIGPQAELRSKDIPVVADLPGVGQNMQDHPACLSAFYLKESAGPISVTDELLHTNGRIRARAILKYLLFKKGPLATTGCDHGAFVKTAGQSEPDLQIRFVPGLALDPDGIGSYTAFGKMKDQKWPSGITFQLLGVRPKSRGSVGLRSDDPWDAPKLDIGFLTDKEGADLATLRSGIKLSREIAAEPAFGAYVGNELHPGAAASSDSAIDSFIRDTVHSGNANVGTCSMGVNGNAVVDPSLRVFGIRGLRVADASVIPVIPGGQTGAATVMVAERAAEILLGSNQKQPAAAVPAAQPALA

SEQ ID NO: 9 - protein, 612 aa, Gonium pectorale
MMLGRKPVAPAKGASAARTVRPVRLAGGRRQLVVSAAAAPVDPAEKYDYILVGGGTAGCVLANKLSADGNKKVLVLEAGPSGDSLEVAVPAGIARLFAHPVMDWGMSSLTQKQLVAREIYLARGRLLGGSSGTNATLYHRGTSSDYDSWGLEGWTSKDVLDWFVKAECYGDGPKPYHGNSGSMNVEQPRYQNPLHEEFFRAAAAAGIPANPDFNDWSRPQDGYGEFQVAQNKGQRADTYRTYLKPALSRGNLKVVTGARTTKVHIEKGSSGPRARGVEFATQQFGDRYSAQLAPGGEVLMCTGAVHTPHLLMLSGVGPAAALREHGVDVVADLAGVGANLQDHPAAVVAVRAKPEFEKLSVTSEIYDEKCNIKLGAVAQYLFNRRGPLATTGCDHGAFVRTSGSHSQPDLQMRFVPGCALDPDGVKSYIVFGELKKQGRAWPGGITLQLLAIRAKSKGSIGLKAADPFINPAININYFSDPADLATLKQGVRMARDIARQEPLRKYLQEETFPGERASSDSDIEEYVRRTVHSGNALVGTCAMGTSPAKGAVVSSSDLKVFGVEGLRVVDASVLPQIPGGQTGAATVMVAERAAALLKGQTTMAPSRQPVAA

SEQ ID NO: 10 - protein, 541 aa, Phaeodactylum tricornutum
MYDYIICGGGLAGCVLAERLSQDESKRVLVLEAGGSDYKSLFIRIPAGVLRLFRSKYDWQHETGGEKGCNGRNVFLQRGKILGGSSCTNVCLHHRGSAEDYNSWNIPGWTATDVLPFFKQSQKDETGRDATFHGADGEWVMDEVRYQNPLSKLFLEVGEAAGLGTNDDFNNWSHPQDGVGRFQVSEVNGERCSGATAFLSKAAKRSNVIVRTGTMVRRIDFDETKTAKGITYDLMGDDTCTVPCLKEGGEVLVTGGAIASPQLLMCSGIGPGKHLRSLGIPVVHDNSAVGENLQDHPAAVVSFKTPQKGVSVTSKLRLFGKTNPIPVFQWLFFKSGLLTSTGCDHGAFVRTSDSLEQPDLQIRFLAARALGPDGMTTYTKFRTMKTVEDGYSFQSVACRAKSKGRIRLSSSNSHVKPMIDGGYLSNQDDLATLRAGIKLGRMLGNRPEWGEYLGQEVYPGPDVQTDEEIDEYIRNSLHTANALTGTCKMGTGRGAVVGPDLRVIGVNGVRVADSSVFPCIPGGQTATPTVMIADRAAVFVR

SEQ ID NO: 11 - protein, 629 aa, unidentified
  source 1..629: note = Emiliania huxleyi
MVALFALQLALSPPQARLGSGSARAALRLRGGSGVTGGSLGRGGGSPAIDGEFDYIIVGGGAAGCVLANRLSADPAHRVLLIEAGGDASRDKRAQVPWAFTKLLRSEYDWDFHVEAEAAVNQQEVYLCRGKALGGSSVTNVMLYHRGSPADYDAWEEAGARGWGAKDVLPYYLRVEDYGDGASQYHAVGGHVSVQEVPYQNQLSATFLRAMGQLGFRPNGDFNDWSSPQEGYGRYKVTQRAGRRCTAADGYLAAARERANLVVVTGAQATRLALDSAYDGAGRLQVSGVEFARGDEREPCSVRLARGGEAVLCAGAVQTPHLLLLSGIGPAEHLREVGVPVRADLPGVGSGLQDHPAVVVSYESKKAVAATDDALLKGYASLVNPLAMLRWLLFGRGPLACAACDHGGFVRSSPDLDQPDVQIRFVPARASSASGMNTLIELGRRARFLPGFSTQVVACRPRSEGRVRLRSADPFAKPIIEGIHLGAAEDVASLRHGIRLGRQVCAAAAFDEYRGEEVFPGAAVQSDEQIDEYIRSSVHSANALTSSCRMGDPSDPAAVLDSHLRVRGVGGLRVADASAMPRIIGGQTQAPTYMLAERAADILLHARLQAHEPATESVSQRLEVAAAAL

SEQ ID NO: 12 - protein, 640 aa, unidentified
  source 1..640: note = Emiliania huxleyi
MSARWLLLLATHCSAALRNPFRAAPTHFDYIIVGGGTAGCVLADRLSAASKQVLVLEPGPSPAAELKIAAPVALTKLFGSEYDWGFRSAPAPGTAGREVHLCRGKCLGGSSATNALLYLRGTAADFDGWGLDGWGSEAMLASFLAVEAQRDAAFRTDALHHGSGGAVPAETPRYANPLSERFLEAAAQAGHPSNADFNDWSRPQAGVGRFQLTTRRGRRAHSAATHLRRAARRPNLHVRCGCAATRLLLEAEGGGGGGGGGGKTRPWTGPAVTGQAGRRAVGVEYIDAAGVQRTASVSGGGGGGGGEVLLCAGAVSSPHLLLLSGIGSPDELAAHGIGAEVCLPGVGRNLIDQPAVVTGYTVTSPLSITDEMFWRRSGALSPRRVGEWLLRGSGPLASSGCDFGGFFSSRPGLAQPDLQLRFVPGLGTSPDGVSSYRDIGRAGKTPSGLTLQSIAVRPTARGSVSLSSADPSAPPRIETGYGTSEADLATLRQGLRLSRELVAQPAFDGVRGEEAWPRAACRLRRPGDDAALDEYIRSTAHSANALGGSCRMGRATSPARLVEGSDPLAVVDPALRVRGASGLRVVDASVLPTLPGGQLGATTFALAERAARIILGERAAGEAEAPAERRQEHAHALGAA

SEQ ID NO: 13 - protein, 1265 aa, unidentified
  source 1..1265: note = Nannochloropsis gaditana
MSSNGYLRAYHLLIALLISANAFLITPPRLSKTTIGLQSFVTANYGVRRAISLRGGLQSVSMKAPAAVASSTYDYIIVGGGIGGCVLANRLTESGRFKVLLLEAGKSAERNPYVNIPAGVVRLFKSALDWQFESAPERHLDGKEVYLVRGKAMGGSSAVNVMLVHRGSASDYAKWEAEGAQGWGPEEALRYFKKMEDNLVGGEGRWHGQGGMYPVDDVKYQNPLSKRFLQACEEYGWRANPDFNDWSHPQDGYGSFKVAQKHGKRVTAASGYLNKAVRRRPNLDILSEALVTRVLLEGEGDVKAVGVEFTGKDGKTHQVRTTGKAGEVLLAGGAVNSPQLLMLSGIGPEADLQAVGIATKVNRPGVGENLQDHPAVTIAHNITRPISLCDDLFLFHTPVPKPHQVLRWTLTGSGPLTTPGCDHGAFLKTREDLQEPNVQFRFIAGRGSDPDGVRSYIMGGSARPLSGLTLQVVNIRPKSKGKLTLASKDPLKKPRIEVRYLSAAEDLQALRTGMRIGRDLIKQRAFADILDEEVFPGPAAQTDEELDAYIRDSLHTANALVGTCKMGSVEDRNAVVDPECRVIGVGGLRVVDASVMPVIPGGQTGSGTTMLAEKAADLVRAHAGDLVEMGVQDEERKGGWFNGLLGRKQKVATEKERGERGKSERFVSEVIRHMGRVFVQVSRARRAQTCMRVGKGLDRERQLECAMRKELTIALFYAMLFTMRHSGFLSTTGRASYKDLGYLTGSCRAHPCTSPSSLCLFPEKPFMKLSPALAVVGFCFNSINVQGFLLSNLAGRSLKHPVPQKGLYSRIEYDAREPRLDEFGLPLDPADLMEKPRVPLKDRVYHIIDMTNDWVDAVSRGRREEETRRIIQRRRAAAKAMAIKDKVLISLDYVFHPVKAWRTFVADPLEARHQRQLRQQAEKRARLERYLQRYNTVKNRFHDTLDLLESTTRTSVKVAKSVSSAVVGAPGTVTRTVKEVKSQAQGTAEAVAKVSSSVSSVVSKITSVIRKEDGALAGAKGKKDPRSEDEGKADPVKVREIWETKEQTAIRTIWEADELVTPVTPPATAMASTVSVSEPQDENEASISQGAAPSPSTSSPSSPEPVTRLSFRARVEADEKERFGSRRLKISGNVPPTASPTRGASSLPLDTLSSSATQTFERSKVGPPIRTSKARCIGKCVHNGWKGICEEWFVHISFPTYAVSIVRPPMHVHNFKVICCVLAVRHARRKKEMSTALSTHLIYLLLKTVKMLQDLPQLRRKGKTN

SEQ ID NO: 14 - protein, 613 aa, Volvox sp.
  source 1..613: note = Volvox carteri f. nagariensis
MLLGQRPFGAPAKGAMPCWKAARHGGVAGVARRPVAVKAAASVGSEKFDYILVGGGTAGCVLANKLSANGSKKVLVLEAGPTGDAMEVAVPAGIARLFAHPVFDWGMSSLTQQQLVAREIYLARGRLLGGSSGTNATLYHRGTPADYDSWGLEGWTSKDLLDWFVKAECYGDGPRAFHGQSGSMNVEQPRYQNVLHDEFFRAAAAAGLPANEDFNDWSRPQEGYGEFQVAQKNGERADTYRTYLKPAMGRDNLKVMTGARTTKVHIEKSSTGPRARGVEYATQQFGERYTAELTPGGEVLMCTGAVHTPHLLMLSGIGPAPTLLEHGLDVISSLPGVGANLQDHPAAVLAVRAKPEFEGLSVTSEIYDSKCNIRLGAVMKYLFGRRGPLATTGCDHGAFVRTSASHSQPDLQMRFVPGCALDPDGVKSYIVFGELKKQGRAWPGGITLQLLGIRAKSRGSIGLKAADPFINPAININYFSDPEDLATLKNGVRIAREIVAQEPLRKYLLEETFPGERANTDKDIEEYVRRTVHSGNALVGTCAMGTTPASGAVVSSADLKVFGVDGLRVVDASVLPRIPGGQTGAATVMVAERAAAMLLGQATITSRREPAAV

SEQ ID NO: 15 - protein, 15 aa, synthetic construct
  VARIANT 2: note = P can be replaced by L or G
  VARIANT 4: note = T can be replaced by A
  VARIANT 5: note = T can be replaced by S or C
  VARIANT 15: note = K can be replaced by R
  VARIANT 12: note = A can be replaced by G
  VARIANT 9: note = D can be replaced by E
  VARIANT 14: note = L can be replaced by V, A or F
  VARIANT 10: note = H can be replaced by N or R
  VARIANT 6: note = P can be replaced by T or A
  VARIANT 7: note = G can be replaced by A
GPLTTPGCDHGAFLK

SEQ ID NO: 16 - protein, 476 aa, Clostridium beijerinckii
MNKDTLIPTTKDLKVKTNGENINLKNYKDNSSCFGVFENVENAISSAVHAQKILSLHYTKEQREKIITEIRKAALQNKEVLATMILEETHMGRYEDKILKHELVAKYTPGTEDLTTTAWSGDNGLTVVEMSPYGVIGAITPSTNPTETVICNSIGMIAAGNAVVFNGHPCAKKCVAFAVEMINKAIISCGGPENLVTTIKNPTMESLDAIIKHPSIKLLCGTGGPGMVKTLLNSGKKAIGAGAGNPPVIVDDTADIEKAGRSIIEGCSFDNNLPCIAEKEVFVFENVADDLISNMLKNNAVIINEDQVSKLIDLVLQKNNETQEYFINKKWVGKDAKLFLDEIDVESPSNVKCIICEVNANHPFVMTELMMPILPIVRVKDIDEAIKYAKIAEQNRKHSAYIYSKNIDNLNRFEREIDTTIFVKNAKSFAGVGYEAEGFTTFTIAGSTGEGITSARNFTRQRRCVLAGLEHHHHHH

SEQ ID NO: 17 - protein, 464 aa, Salmonella sp.
  source 1..464: note = Salmonella typhimurium
MNTSELETLIRTILSEQLTTPAQTPVQPQGKGIFQSVSEAIDAAHQAFLRYQQCPLKTRSAIISAMRQELTPLLAPLAEESANETGMGNKEDKFLKNKAALDNTPGVEDLTTTALTGDGGMVLFEYSPFGVIGSVAPSTNPTETIINNSISMLAAGNSIYFSPHPGAKKVSLKLISLIEEIAFRCCGIRNLVVTVAEPTFEATQQMMAHPRIAVLAITGGPGIVAMGMKSGKKVIGAGAGNPPCIVDETADLVKAAEDIINGASFDYNLPCIAEKSLIVVESVAERLVQQMQTFGALLLSPADTDKLRAVCLPEGQANKKLVGKSPSAMLEAAGIAVPAKAPRLLIALVNADDPWVTSEQLMPMLPVVKVSDFDSALALALKVEEGLHHTAIMHSQNVSRLNLAARTLQTSIFVKNGPSYAGIGVGGEGFTTFTIATPTGEGTTSARTFARSRRCVLTNGFSIR

SEQ ID NO: 18 - protein, 521 aa, Aeromonas hydrophila
  source 1..521: note = subsp. hydrophila
MLSRQNARELVRNAKQAQVIMATFSQQKIDAIVKNVAEEAARHAETLAKMAAEETGFGNWQDKVLKNRFASLHVYDAIKEMKTVGIIHDDQAKKVMDVGVPLGVICALVPSTNPTSTIFYKTLIALKAGNAIIFSPHPGARQCSWKAIEIVKRAAEAAGAPAGIVDGVTQLTLEATSELMHSKDVSLILATGGEGMVRAAYASGTPTISGGPGNGPAFIERSADIHQAVKDIITSKTFDNGVICASEQSIIVERCIYDEVHRELAAQGAYFMNEDEAARMAALLLRPNGTINPKVVGKTALHLSQLAGFSVPPSTRVLVAEQTTVSHSNPYSREKLCPVLGLYVEEEWRAACHRVVELLTNEGLGHTLVIHTRNQDVIRQFSLEKPVNRILINTPAALGGIGATTNLTPALTLGCGAVGGGSSSDNVGPMNLLNIRKVGYGVRTIEELRAPIQPVAVQPASAAPTAPQPCSILDDARFSAPAPACHSADDRFAGASAEVGGEISEQNVERVIRQVLERLGK

SEQ ID NO: 19 - protein, 462 aa, Klebsiella pneumoniae
  source 1..462: note = subsp. rhinoscleromatis
MNTAELETLIRTILSEKLAPTPPAPQQEQGIFCDVGSAIDAAHQAFLRYQQCPLKTRSAIISALRETLAPELATLAEESATETGMGNKEDKYLKNKAALENTPGIEDLTTSALTGDGGMVLFEYSPFGVIGAVAPSTNPTETIINNSISMLAAGNSVYFSPHPGAKKVSLKLIARIEEIAYRCSGIRNLVVTVAEPTFEATQQMMSHPLIAVLAITGGPGIVAMGMKSGKKVIGAGAGNPPCIVDETADLVKAAEDIISGAAFDYNLPCIAEKSLIVVASVADRLIQQMQDFDALLLSRQEADTLRAVCLPDGAANKKLVGKSPAALLAAAGLAVPPRPPRLLIAEVEANDPWVTCEQLMPVLPIVRVADFDSALALALRVEEGLHHTAIMHSQNVSRLNLAARTLQTSIFVKNGPSYAGIGVGGEGFTTFTIATPTGEGTTSARTFARLRRCVLTNGFSIR

SEQ ID NO: 20 - protein, 477 aa, Lactobacillus brevis
MNTENIEQAIRKILSEELSNPQSSTATNTTVPGKNGIFKTVNEAIAATKAAQENYADQPISVRNKVIDAIREGFRPYIEDMAKRIHDETGMGTVSAKIAKLNNALYNTPGPEILQPEAETGDGGLVMYEYAPFGVIGAVGPSTNPSETVIANAIMMLAGGNTLFFGAHPGAKNITRWTIEKLNELVADATGLHNLVVSLETPSIESVQEVMQHPDVAMLSITGGPAVVHQALISGKKAVGAGAGNPPAMVDATANIALAAHNIVDSAAFDNNILCTAEKEVVVEAAVKDELIMRMQQEGAFLVTDSADIEKLAQMTIGPKGAPDRKFVGKDATYILDQAGISYTGTPTLIILEAAKDHPLVTTEMLMPILPVVCCPDFDSVLATATEVEGGLHHTASIHSENLPHINKAAHRLNTSIFVVNGPTYCGTGVATNGAHSGASALTIATPTGEGTATSKTYTRRRRLNSPEGFSLRTWEA

SEQ ID NO: 21 - protein, 469 aa, Listeria monocytogenes
MESLELEKLVKKVLLEKLAEQKGIPVKTMTKGAKSGVFDTVDEAVQAAVIAQNSYKEKSLEERRNVVKAIREALYPEIESIAARAVAETGMGNVADKILKNTLAIEKTPGVEDLYTEVATGDNGMTLYELSPYGVIGAVAPSTNPTETLICNTIGMLAAGNAVFYSPHPGAKNISLWLIEKLNTIVRESCGVDNLVVTVEKPSIQAAQEMMNHPKVPLLVITGGPGVVLQAMQSGKKVIGAGAGNPPSIVDETANIEKAAADIVDGASFDHNILCIAEKSVVAVDSIADFLMFQMEKNGALHVTNPSDIQKLEKVAVTDKGVTNKKLVGKSASEILKEAGIACDFSPRLIIVETEKTHPFATVELLMPIVPVVRVPNFEEALEVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 22 - protein, 451 aa, Porphyromonas gingivalis
MEIKEMVSLARKAQKEYQATHNQEAVDNICRAAAKVIYENAAILAREAVDETGMGVYEHKVAKNQGKSKGVWYNLHNKKSVGILNIDERTGMIEIAKPIGVVGAVTPTTNPIVTPMSNIIFALKTCNAIIIAPHPRSKKCSAHAVRLIKEAIAPFNVPEGMVQIIEEPSIEKTQELMGAVDVVVATGGMGMVKSAYSSGKPSFGVGAGNVQVIVDSNIDFEAAAEKIITGRAFDNGIICSGEQSIIYNEADKEAVFTAFRNHGAYFCDEAEGDRARAAIFENGAIAKDVVGQSVAFIAKKANINIPEGTRILVVEARGVGAEDVICKEKMCPVMCALSYKHFEEGVEIARTNLANEGNGHTCAIHSNNQAHIILAGSELTVSRIVVNAPSATTAGGHIQNGLAVTNTLGCGSWGNNSISENFTYKHLLNISRIAPLNSSIHIPDDKEIWEL

SEQ ID NO: 23 - protein, 243 aa, Prochlorococcus marinus
MPTLEMPVAAVLDSTVGSSEALPDFTSDRYKDAYSRINAIVIEGEQEAHDNYIAIGTLLPDHVEELKRLAKMEMRHKKGFTACGKNLGVEADMDFAREFFAPLRDNFQTALGQGKTPTCLLIQALLIEAFAISAYHTYIPVSDPFARKITEGVVKDEYTHLNYGEAWLKANLESCREELLEANRENLPLIRRMLDQVAGDAAVLQMDKEDLIEDFLIAYQESLTEIGFNTREITRMAAAALVS

SEQ ID NO: 24 - protein, 394 aa, Escherichia coli
MKNCVIVSAVRTAIGSFNGSLASTSAIDLGATVIKAAIERAKIDSQHVDEVIMGNVLQAGLGQNPARQALLKSGLAETVCGFTVNKVCGSGLKSVALAAQAIQAGQAQSIVAGGMENMSLAPYLLDAKARSGYRLGDGQVYDVILRDGLMCATHGYHMGITAENVAKEYGITREMQDELALHSQRKAAAAIESGAFTAEIVPVNVVTRKKTFVFSQDEFPKANSTAEALGALRPAFDKAGTVTAGNASGINDGAAALVIMEESAALAAGLTPLARIKSYASGGVPPALMGMGPVPATQKALQLAGLQLADIDLIEANEAFAAQFLAVGKNLGFDSEKVNVNGGAIALGHPIGASGARILVTLLHAMQARDKTLGLATLCIGGGQGIAMVIERLN

SEQ ID NO: 25 - protein, 329 aa, Streptomyces sp.
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHHLGATGTAAFDVNAVCSGTVFALSSVAGTLVYRGGYALVIGADLYSRILNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAASFALIEW

SEQ ID NO: 26 - protein, 282 aa, Clostridium acetobutylicum
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKRPDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSK

SEQ ID NO: 27 - protein, 261 aa, Clostridium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 28 - protein, 397 aa, Treponema denticola
MIVKPMVRNNICLNAHPQGCKKGVEDQIEYTKKRITAEVKAGAKAPKNVLVLGCSNGYGLASRITAAFGYGAATIGVSFEKAGSETKYGTPGWYNNLAFDEAAKREGLYSVTIDGDAFSDEIKAQVIEEAKKKGIKFDLIVYSLASPVRTDPDTGIMHKSVLKPFGKTFTGKTVDPFTGELKEISAEPANDEEAAATVKVMGGEDWERWIKQLSKEGLLEEGCITLAYSYIGPEATQALYRKGTIGKAKEHLEATAHRLNKENPSIRAFVSVNKGLVTRASAVIPVIPLYLASLFKVMKEKGNHEGCIEQITRLYAERLYRKDGTIPVDEENRIRIDDWELEEDVQKAVSALMEKVTGENAESLTDLAGYRHDFLASNGFDVEGINYEAEVERFDRI

SEQ ID NO: 29 - protein, 154 aa, Haemophilus influenzae
MSANFTDKNGRQSKGVLLLRTLAMPSDTNANGDIFGGWIMSQMDMGGAILAKEIAHGRVVTVAVESMNFIKPISVGDVVCCYGQCLKVGRSSIKIKVEVWVKKVASEPIGERYCVTDAVFTFVAVDNNGRSRTIPRENNQELEKALALISEQPL

SEQ ID NO: 30 - protein, 329 aa, Streptomyces clavuligerus
MQEFEFAVPAPSRVSPDLARARARHLDWVHAMDLVRGEEARRRYEFSCVADIGAYGYPHATGADLDLCVDVLGWTFLFDDQFDAGDGRERDALAVCAELTDLLWKGTAATAASPPIVVAFSDCWERMRAGMSDAWRRRTVHEWVDYLAGWPTKLADRAHGAVLDPAAHLRARHRTICCRPLFALAERVGGYEVPRRAWHSSRLDGMRFTTSDAVIGMNELHSFEKDRAQGHANLVLSLVHHGGLTGPEAVTRVCDLVQGSIESFLRLRSGLPELGRALGVEGAVLDRYADALSAFCRGYHDWGRGASRYTTRDHPGDLGLENLVARSSG

SEQ ID NO: 31 - protein, 329 aa, synthetic construct
MQEFEFAVPAPSRVSPDLARARARHLDWVHAMDLVRGEEARRRYEFSCVADIGAYGYPHATGADLDLCVDVMGWTFLFDDQFDAGDGRERDALAVCAELTDLLWKGTAATAASPPIVVAFSDCWERMRAGMSDAWRRRTVHEWVDYLAGWPTKLADRAHGAVLDPAAHLRARHRTICCRPLFALAERVGGYEVPRRAWHSSRLDGMRFTTSDAVIGMNELHSFEKDRAQGHANLVLSLVHHGGLTGPEAVTRVCDLVQGSIESFLRLRSGLPELGRALGVEGAVLDRYADALSAFCRGYHDWGRGASRYTTRDHPGDLGLENLVARSSG

SEQ ID NO: 32 - protein, 329 aa, synthetic construct
MQEFEFAVPAPSRVSPDLARARARHLDWVHAMDLVRGEEARRRYEFSCVADIGAYGYPHATGADLDLCVDVLGWTFLFDDQFDAGDGRERDALAVCAELTDLLWKGTAATAASPPIVVAFSDCWERMRAGMSDAWRRRTVHEWVDYLAGWPTKLADRAHGAVLDPAAHLRARHRTICCRPLFALAERVGGYEVPRRAWHSSRLDGMRFTTSDALIGMNELHSFEKDRAQGHANLVLSLVHHGGLTGPEAVTRVCDLVQGSIESFLRLRSGLPELGRALGVEGAVLDRYADALSAFCRGYHDWGRGASRYTTRDHPGDLGLENLVARSSG

SEQ ID NO: 33 - protein, 329 aa, synthetic construct
MQEFEFAVPAPSRVSPDLARARARHLDWVHAMDLVRGEEARRRYEFSCVADIGAYGYPHATGADLDLCVDVLGWTFLFDDQFDAGDGRERDALAVCAELTDLLWKGTAATAASPPIVVAFSDCWERMRAGMSDAWRRRTVHEWVDYLAGWPTKLADRAHGAVLDPAAHLRARHRTICCRPLFALAERVGGYEVPRRAWHSSRLDGMRFTTSDAIIGMNELHSFEKDRAQGHANLVLSLVHHGGLTGPEAVTRVCDLVQGSIESFLRLRSGLPELGRALGVEGAVLDRYADALSAFCRGYHDWGRGASRYTTRDHPGDLGLENLVARSSG

SEQ ID NO: 34 - DNA, 32 bp, synthetic construct
  source 1..32: mol_type = other DNA
aggagatataccatgcctacgggnggcwgcag

SEQ ID NO: 35 - DNA, 33 bp, synthetic construct
  source 1..33: mol_type = other DNA
tggtggtgctcgaggactacgggtatctaatcc

SEQ ID NO: 36 - DNA, 19 bp, synthetic construct
  source 1..19: mol_type = other DNA
ctcgagcaccaccaccacc

SEQ ID NO: 37 - DNA, 45 bp, synthetic construct
  source 1..45: mol_type = other DNA
ctctagaaataattttgtttaactttaagaaggagatataccatg

SEQ ID NO: 38 - protein, 97 aa, Synechocystis sp.
MASYTVKLITPDGESSIECSDDTYILDAAEEAGLDLPYSCRAGACSTCAGKITAGSVDQSDQSFLDDDQIEAGYVLTCVAYPTSDCTIETHKEEDLY

SEQ ID NO: 39 - protein, 381 aa, Abies grandis
MAYSAMATMGYNGMAASCHTLHPTSPLKPFHGASTSLEAFNGEHMGLLRGYSKRKLSSYKNPASRSSNATVAQLLNPPQKGKKAVEFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQN

SEQ ID NO: 40 - protein, 297 aa, synthetic construct
MEFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQN

SEQ ID NO: 41 - protein, 1174 aa, Mycobacterium marinum
MSPITREERLERRIQDLYANDPQFAAAKPATAITAAIERPGLPLPQIIETVMTGYADRPALAQRSVEFVTDAGTGHTTLRLLPHFETISYGELWDRISALADVLSTEQTVKPGDRVCLLGFNSVDYATIDMTLARLGAVAVPLQTSAAITQLQPIVAETQPTMIAASVDALADATELALSGQTATRVLVFDHHRQVDAHRAAVESARERLAGSAVVETLAEAIARGDVPRGASAGSAPGTDVSDDSLALLIYTSGSTGAPKGAMYPRRNVATFWRKRTWFEGGYEPSITLNFMPMSHVMGRQILYGTLCNGGTAYFVAKSDLSTLFEDLALVRPTELTFVPRVWDMVFDEFQSEVDRRLVDGADRVALEAQVKAEIRNDVLGGRYTSALTGSAPISDEMKAWVEELLDMHLVEGYGSTEAGMILIDGAIRRPAVLDYKLVDVPDLGYFLTDRPHPRGELLVKTDSLFPGYYQRAEVTADVFDADGFYRTGDIMAEVGPEQFVYLDRRNNVLKLSQGEFVTVSKLEAVFGDSPLVRQIYIYGNSARAYLLAVIVPTQEALDAVPVEELKARLGDSLQEVAKAAGLQSYEIPRDFIIETTPWTLENGLLTGIRKLARPQLKKHYGELLEQIYTDLAHGQADELRSLRQSGADAPVLVTVCRAAAALLGGSASDVQPDAHFTDLGGDSLSALSFTNLLHEIFDIEVPVGVIVSPANDLQALADYVEAARKPGSSRPTFASVHGASNGQVTEVHAGDLSLDKFIDAATLAEAPRLPAANTQVRTVLLTGATGFLGRYLALEWLERMDLVDGKLICLVRAKSDTEARARLDKTFDSGDPELLAHYRALAGDHLEVLAGDKGEADLGLDRQTWQRLADTVDLIVDPAALVNHVLPYSQLFGPNALGTAELLRLALTSKIKPYSYTSTIGVADQIPPSAFTEDADIRVISATRAVDDSYANGYSNSKWAGEVLLREAHDLCGLPVAVFRCDMILADTTWAGQLNVPDMFTRMILSLAATGIAPGSFYELAADGARQRAHYDGLPVEFIAEAISTLGAQSQDGFHTYHVMNPYDDGIGLDEFVDWLNESGCPIQRIADYGDWLQRFETALRALPDRQRHSSLLPLLHNYRQPERPVRGSIAPTDRFRAAVQEAKIGPDKDIPHVGAPIIVKYVSDLRLLGLL

SEQ ID NO: 42 - DNA, 675 bp, Bacillus subtilis
  source 1..675: mol_type = genomic DNA
atgaagatttacggaatttatatggaccgcccgctttcacaggaagaaaatgaacggttcatgtctttcatatcacctgaaaaacgggagaaatgccggagattttatcataaagaagatgctcaccgcaccctgctgggagatgtgctcgttcgctcagtcataagcaggcagtatcagttggacaaatccgatatccgctttagcacgcaggaatacgggaagccgtgcatccctgatcttcccgacgctcatttcaacatttctcactccggacgctgggtcatttgcgcgtttgattcacagccgatcggcatagatatcgaaaaaacgaaaccgatcagccttgagatgcccaagcgcttcttttcaaaaacagagtacagcgaccttttagcaaaagacaaggacgagcagacagactatttttatcatctatggtcaatgaaagaaagctttatcaaacaaggaaggcaaaggcttatcgcttccgcttgattccttttcagtgcgcctgcaccaggacggacaagtatccattgagcttccggacagccattccccatgctatatcaaaacgtatgaggtcgatcccggctacaaaatggctgtatgcgccgtacaccctgatttccccgaggatatcacaatggtctcgtacgaagagcttttata

SEQ ID NO: 43 - protein, 1174 aa, Nocardia iowensis
MAVDSPDERLQRRIAQLFAEDEQVKAARPLEAVSAAVSAPGMRLAQIAATVMAGYADRPAAGQRAFELNTDDATGRTSLRLLPRFETITYRELWQRVGEVAAAWHHDPENPLRAGDFVALLGFTSIDYATLDLADIHLGAVTVPLQASAAVSQLIAILTETSPRLLASTPEHLDAAVECLLAGTTPERLVVFDYHPEDDDQRAAFESARRRLADAGSLVIVETLDAVRARGRDLPAAPLFVPDTDDDPLALLIYTSGSTGTPKGAMYTNRLAATMWQGNSMLQGNSQRVGINLNYMPMSHIAGRISLFGVLARGGTAYFAAKSDMSTLFEDIGLVRPTEIFFVPRVCDMVFQRYQSELDRRSVAGADLDTLDREVKADLRQNYLGGRFLVAVVGSAPLAAEMKTFMESVLDLPLHDGYGSTEAGASVLLDNQIQRPPVLDYKLVDVPELGYFRTDRPHPRGELLLKAETTIPGYYKRPEVTAEIFDEDGFYKTGDIVAELEHDRLVYVDRRNNVLKLSQGEFVTVAHLEAVFASSPLIRQIFIYGSSERSYLLAVIVPTDDALRGRDTATLKSALAESIQRIAKDANLQPYEIPRDFLIETEPFTIANGLLSGIAKLLRPNLKERYGAQLEQMYTDLATGQADELLALRREAADLPVLETVSRAAKAMLGVASADMRPDAHFTDLGGDSLSALSFSNLLHEIFGVEVPVGVVVSPANELRDLANYIEAERNSGAKRPTFTSVHGGGSEIRAADLTLDKFIDARTLAAADSIPHAPVPAQTVLLTGANGYLGRFLCLEWLERLDKTGGTLICVVRGSDAAAARKRLDSAFDSGDPGLLEHYQQLAARTLEVLAGDIGDPNLGLDDATWQRLAETVDLIVHPAALVNHVLPYTQLFGPNVVGTAEIVRLAITARRKPVTYLSTVGVADQVDPAEYQEDSDVREMSAVRVVRESYANGYGNSKWAGEVLLREAHDLCGLPVAVFRSDMILAHSRYAGQLNVQDVFTRLILSLVATGIAPYSFYRTDADGNRQRAHYDGLPADFTAAAITALGIQATEGFRTYDVLNPYDDGISLDEFVDWLVESGHPIQRITDYSDWFHRFETAIRALPEKQRQASVLPLLDAYRNPCPAVRGAILPAKEFQAAVQTAKIGPEQDIPHLSAPLIDKYVSDLELLQLL

SEQ ID NO: 44 - protein, 1162 aa, Mycobacterium phlei
MASESRDVRLQRRIAELYDTDPQFAAARPDEAVARAVNAPGLTLSQVIRTVLDNYADRPALGYRAVEFAADPASARTVARLLPRFDTITYRELGERIDATTAALGHDGLRPGERVAILGFSSVDYTTIDIAAFNLGAVSVPLQTSAPPSQLRPMTAETEPAVIAASVDFLDDALELIRTGHAPRRLVVFDFHPEIDDHRDALAAATTGLADTAMTVETLDSLLTRGRTLPAPTGYRDRDDDLALLIYTSGSTGAPKGAMYQRRMVTNMWRRATTAIWGGKEAAPWLTLNFMPMSHVMGRGILSTTLCSGGTAYFAARSDLSTLLEDLALVRPTQLTFVPRIWEMIFQEYQRDVARRPEAEVLADLREHRLGGRFLAAMTGSAPMSPEMTAFAESVLDLHLVDGYGSTEAGSIVLDGQVLRPPVLDYKLVDVPELGYFSTDRPYPRGELLVKTELMFPGYYKRPDITAEVFDEDGYYRTGDIVAELGPDRLAYVDRRNNVLKLSQGEFVTVSKLEAAFAASPLVRQIYIYGNSAHPYLLAVVVPTEDALTRYDAATLKTAISESLQDVGRAAGLQSYEIPRDFLVETTPFTLENGLLTGLRKLARPKLKEHYGERLEQLYTELADGQAEELKELRTHGAQQPTLTTVSRAATALLGTASAEVRPDAHFTDLGGDSLSALTFGNLLGEIYAVEVPVGVIVSPANDLAAIADYIDTARRPGDGRPTFAGVHGDDAAEVHARDLTLDRFLDDATLAAATALPGPAPEIRTVLLTGATGFLGRYLALEWLERMAMVGGTLICLVRGRDDAAARARLDQIFDSGDPELLRHYRELADRHLEVIAGDKSDAVLGLDRRTWQRLADTVDLIVDPAALVNHVLPYRELFGPNVVGTAELIRLALTGRLKPYLYTSTIAVGAGIAPGQFTEDADIRQISATRTLDDSYANGYATSKWAGEVLLREAHDLCGLPVAVFRCDMILADTSYAGQLNVPDMFTRLILSLVATGIAPLSFYELDAAGHRQRAHYDGLPVEFVAEAVSALGLDVAEDGGFATYHVMNPYDDGIGLDEFVDWLTGAGYPIERVNDYGTWFQRFETAIRGLPERQRQASLLPLLHNYQRPQPPIRGSAAPTDRFRSAVQDAKIGPDKDIPHITPEVIVKYVTDLRLLGLL

SEQ ID NO: 45 - protein, 1168 aa, Mycobacterium smegmatis
MTIETREDRFNRRIDHLFETDPQFAAARPDEAISAAAADPELRLPAAVKQILAGYADRPALGKRAVEFVTDEEGRTTAKLLPRFDTITYRQLAGRIQAVTNAWHNHPVNAGDRVAILGFTSVDYTTIDIALLELGAVSVPLQTSAPVAQLQPIVAETEPKVIASSVDFLADAVALVESGPAPSRLVVFDYSHEVDDQREAFEAAKGKLAGTGVVVETITDALDRGRSLADAPLYVPDEADPLTLLIYTSGSTGTPKGAMYPESKTATMWQAGSKARWDETLGVMPSITLNFMPMSHVMGRGILCSTLASGGTAYFAARSDLSTFLEDLALVRPTQLNFVPRIWDMLFQEYQSRLDNRRAEGSEDRAEAAVLEEVRTQLLGGRFVSALTGSAPISAEMKSWVEDLLDMHLLEGYGSTEAGAVFIDGQIQRPPVIDYKLVDVPDLGYFATDRPYPRGELLVKSEQMFPGYYKRPEITAEMFDEDGYYRTGDIVAELGPDHLEYLDRRNNVLKLSQGEFVTVSKLEAVFGDSPLVRQIYVYGNSARSYLLAVVVPTEEALSRWDGDELKSRISDSLQDAARAAGLQSYEIPRDFLVETTPFTLENGLLTGIRKLARPKLKAHYGERLEQLYTDLAEGQANELRELRRNGADRPVVETVSRAAVALLGASVTDLRSDAHFTDLGGDSLSALSFSNLLHEIFDVDVPVGVIVSPATDLAGVAAYIEGELRGSKRPTYASVHGRDATEVRARDLALGKFIDAKTLSAAPGLPRSGTEIRTVLLTGATGFLGRYLALEWLERMDLVDGKVICLVRARSDDEARARLDATFDTGDATLLEHYRALAADHLEVIAGDKGEADLGLDHDTWQRLADTVDLIVDPAALVNHVLPYSQMFGPNALGTAELIRIALTTTIKPYVYVSTIGVGQGISPEAFVEDADIREISATRRVDDSYANGYGNSKWAGEVLLREAHDWCGLPVSVFRCDMILADTTYSGQLNLPDMFTRLMLSLVATGIAPGSFYELDADGNRQRAHYDGLPVEFIAEAISTIGSQVTDGFETFHVMNPYDDGIGLDEYVDWLIEAGYPVHRVDDYATWLSRFETALRALPERQRQASLLPLLHNYQQPSPPVCGAMAPTDRFRAAVQDAKIGPDKDIPHVTADVIVKYISNLQMLGLL

SEQ ID NO: 46 - protein, 1173 aa, Mycobacterium smegmatis
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 47 - protein, 1156 aa, Nocardia otitidiscaviarum
MLDDARAERRERRIADALADDQVREAVADAAVSESVRRVEVRLARIVDAVMSGYGDRAALAWRRSELVDGAVRLLPEYSTMTYRELWRRAGAVAAEWGADAESPVRAGDFVCTLGFTSPDYTVVDLALVRLGAVAVPLQASASVAQWRSIMAETEPRMLATSAETLPAAVEAVLGGFAPRRVLVFDYRPELEAHRSAVDSARERLAEVGCRVATVADAVDRGASLPAPLCIPSDRERLALLIYTSGSTGAPKGAMYTDRLAAGLWLSANDIRVPALTMNYMPLSHVAGRMSLYGTLMRGGTAYFAAASDMSTLLDDFGLARPTELFLVPRVCELLHQRYQSELDRRVLAGDDVETTATDVKAELRERVLGGRYLTALSGSAPLAAEMKTFMESLLDDELHDGYGSTEAGGSVLLDNRIKRPPVLDYRLVDVPELGYFRTDKPHPRGELLLKTESMFPGYYKRPEITAEMFDADGFYRTGDVVAELGPDQLVYVDRRNNVLKLSQGEFVTVAALEAVYATSPLIRQIFVYGSSERAYLLAVVVPTDEVLALPPARARAEVGESLHRIAKESGLQPYEIPREVLIESEPFTIDNGLLSGIGKLLRPKLKEHYGEQLERLYTELAQQREDELTTLRRGAHERPVLETVTRAAGAVLDLAAGEVSPDAHFADLGGDSLSALSFSTLLRDIFGVEVPVGVIVGPATDLTRIAEYLVAERDSGTRPTAATVHGDDGLLRADDLTLEAFLDPGTLAAATRLSAPTDPPRTVLLTGANGYLGRFLALEWLQRLDVSGGTLICLVRGSDADAARRRLDAVFATGDPELEAHYRDLAGRRLRVLAGDIGEPNLGLSEQDWRDLAESVDVIVHPAALVNHVLPYAQLFGPNVVGTAEVIRLALTARLKPVTYLSTVAVSAGIDPETFTEDGDIREISPVRRLDDGYANGYGNSKWAGEVLLRAAHDRFGLPVAVFRSDMILAHSHYAGQLNVPDMFTRLLLSVLATGLAPGSFHDAHGERHRAHYDGLPADFTAAAVTTLGAQATSGFDTYDVLNPHDDGLSLDTFIDWLIDSGHPIDRIDDYADWFTRFETAVRALPEPQRQHSLLPLLHAYRHPTPPLRGAALPAKHFRAAVQQAKLGPDADIPHLTRELIEKYVSDLRLLGLLG

SEQ ID NO: 48 - protein, 1105 aa, Tsukamurella paurometabola
MSIETVQNGVPAEGSVPPADQQTERLPQVIARIFAQFADRPAFATREAGPGTPYATVSYREIWRRVTALVASWQSEVAPGDFVAILGFTSSDFVTVDLATTLLGAPNVPLQAGAPAARIATILDETRPKILAVSADQVDLAQEALAESAATPRVVVFDGERDGYEGIEADILSGSALPAPEFFAPEPGTDPLVTLIYTSGSTGTPKGAMYTEQLVRDAWLKVDSIVDIDMPAESLLHFLPMSHMYGRNWLIAGLASGGTGYFAGASDMSTLFDDLAAARPTAIGLVPRVCELIHQRYLAVEADTDAETARVELRDRVLGGRLQAAMCGSAALSSELQTFMEWLLGIDIQIGYGSTEAGGVIRDGVVVRPPVTEYKLIDVPELGYFVTDSPHPRGELLVKSTQLIPGYYNSDKRIRDDEGFYRTGDVMAELGPDRLEYVDRRSNVIKLAQGEFVPIAQLEAIYAAGPDVHQIFLYGTSERSYLIGVVVPAPGPDGETDAQTRTRVLDGLAAIARENDLAAYEVPRDVLIERDPFSQENGLRSGIGKLVRPALIARYGDRLHDLYAQADTRQREGLRALDASGPIIDTVLGAAALTLGADIADFDADTRFGDLGGDSLSALSLATTLEGLYDVPVPVQTIVGPTATLGGVARHIEKARSGGVAAPTADSVHGVGASVARATDLTLEKFIDPELLALAPTLPAATGEPNTVLLTGSTGYLGRFLLLDWLRRVAPHGGTVIALVRGADADDARRRVTAAIGDSDPDLTQEFTSLAEHHLHVIAGDFGSPALGLDDATWSDLAGRVDHVVHCGALVNHVLPYDQLFGPNVVATGEVVRLALTTRRKSVDYVSTVAVVPQDDGRVLVEDDDVRELGAERRIGADAYANGYAVSKWAGEVLLHEAADLADLPVRVFRSDMILAHSRFHGQFNEVDQFTRLLLSIAETGLAPASFYTPDPSGHRPHYDGLPVDFTAEAITTLSAAGRSGYRTFHVLNANDDGVSLDSFVDWIAASGRSIERIDDYDTWFARFEQALQQLPDEARQRSVLPLLHAVREPAPAAGTSALSVDRFRGAVRETGVGPGDIPVLDRALIEKYLRDFETAGWLAPGARD

SEQ ID NO: 49 - protein, 459 aa, Chromobacterium violaceum
MQKQRTTSQWRELDAAHHLHPFTDTASLNQAGARVMTRGEGVYLWDSEGNKIIDGMAGLWCVNVGYGRKDFAEAARRQMEELPFYNTFFKTTHPAVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPIGAVFVGKRVAEGLIAGGDFNHGFTYSGHPVCAAVAHANVAALRDEGIVQRVKDDIGPYMQKRWRETFSRFEHVDDVRGVGMVQAFTLVKNKAKRELFPDFGEIGTLCRDIFFRNNLIMRACGDHIVSAPPLVMTRAEVDEMLAVAERCLEEFEQTLKARGLA

SEQ ID NO: 50 - protein, 453 aa, Vibrio fluvialis
MNKPQSWEARAETYSLYGFTDMPSLHQRGTVVVTHGEGPYIVDVNGRRYLDANSGLWNMVAGFDHKGLIDAAKAQYERFPGYHAFFGRMSDQTVMLSEKLVEVSPFDSGRVFYTNSGSEANDTMVKMLWFLHAAEGKPQKRKILTRWNAYHGVTAVSASMTGKPYNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPMGAVILGPELSKRLETAIEAIEEFPHGFTASGHPVGCAIALKAIDVVMNEGLAENVRRLAPRFEERLKHIAERPNIGEYRGIGFMWALEAVKDKASKTPFDGNLSVSERIANTCTDLGLICRPLGQSVVLCPPFILTEAQMDEMFDKLEKALDKVFAEVA

SEQ ID NO: 51 - protein, 440 aa, Alcaligenes sp.
  source 1..440: note = Alcaligenes denitrificans
MSAAKLPDLSHLWMPFTANRQFKANPRLLASAKGMYYTSFDGRQILDGTAGLWCVNAGHCREEIVSAIASQAGVMDYAPGFQLGHPLAFEAATAVAGLMPQGLDRVFFTNSGSESVDTALKIALAYHRARGEAQRTRLIGRERGYHGVGFGGISVGGISPNRKTFSGALLPAVDHLPHTHSLEHNAFTRGQPEWGAHLADELERIIALHDASTIAAVIVEPMAGSTGVLVPPKGYLEKLREITARHGILLIFDEVITAYGRLGEATAAAYFGVTPDLITMAKGVSNAAVPAGAVAVRREVHDAIVNGPQGGIEFFHGYTYSAHPLAAAAVLATLDIYRREDLFARARKLSAAFEEAAHSLKGAPHVIDVRNIGLVAGIELSPREGAPGARAAEAFQKCFDTGLMVRYTGDILAVSPPLIVDENQIGQIFEGIGKVLKEVA

SEQ ID NO: 52 - protein, 452 aa, Pseudomonas putida
MSEQNSQTLAWQTMSRDHHLAPFSDVRQLAEKGPRIITSAKGVYLWDSEGNKILDGMAGLWCVAVGYGREELAEVASQQMKQLPYYNLFFQTAHPPALELAKAIAEVAPQGMNHVFFTGSGSEGNDTVLRMVRHYWALKGQKNKKVIIGRINGYHGSTVAGAALGGMSGMHQQGGVIPDVVHIPQPYWFGEGGDMTEADFGVWAAEQLEKKILEVGVDNVAAFIAEPIQGAGGVIIPPQTYWPKIKEILARYDILFVADEVICGFGRTGEWFGTDYYDLKPDLMTIAKGLTSGYIPMGGVIVRDEVAKVISEGGDFNHGFTYSGHPVAAAVGLENLRILRDEQIIQQVHDKTAPYLQQRLRELADHPLVGEVRGLGMLGAIELVKDKATRARHEGKGVGMICRQHCFDNGLIMRAVGDTMIIAPPLVISIEEIDELVEKARKCLDLTYEAVR

SEQ ID NO: 53 - protein, 453 aa, Paracoccus denitrificans
MNQPQSWEARAETYSLYGFTDMPSVHQRGTVVVTHGEGPYIVDVHGRRYLDANSGLWNMVAGFDHKGLIEAAKAQYDRFPGYHAFFGRMSDQTVMLSEKLVEVSPFDNGRVFYTNSGSEANDTMVKMLWFLHAAEGKPQKRKILTRWNAYHGVTAVSASMTGKPYNSVFGLPLPGFIHLTCPHYWRYGEEGETEAQFVARLARELEDTITREGADTIAGFFAEPVMGAGGVIPPAKGYFQAILPILRKYDIPMISDEVICGFGRTGNTWGCLTYDFMPDAIISSKNLTAGFFPMGAVILGPDLAKRVEAAVEAIEEFPHGFTASGHPVGCAIALKAIDVVMNEGLAENVRRLAPRFEAGLKRIADRPNIGEYRGIGFMWALEAVKDKPTKTPFDANLSVSERIANTCTDLGLICRPLGQSIVLCPPFILTEAQMDEMFEKLEKALDKVFAEVA

SEQ ID NO: 54 - protein, 456 aa, Pseudomonas aeruginosa
MNSQITNAKTREWQALSRDHHLPPFTDYKQLNEKGARIITKAEGVYIWDSEGNKILDAMAGLWCVNVGYGREELVQAATRQMRELPFYNLFFQTAHPPVVELAKAIADVAPEGMNHVFFTGSGSEANDTVLRMVRHYWATKGQPQKKVVIGRWNGYHGSTVAGVSLGGMKALHEQGDFPIPGIVHIAQPYWYGEGGDMSPDEFGVWAAEQLEKKILEVGEENVAAFIAEPIQGAGGVIVPPDTYWPKIREILAKYDILFIADEVICGFGRTGEWFGSQYYGNAPDLMPIAKGLTSGYIPMGGVVVRDEIVEVLNQGGEFYHGFTYSGHPVAAAVALENIRILREEKIIEKVKAETAPYLQKRWQELADHPLVGEARGVGMVAALELVKNKKTRERFTDKGVGMLCREHCFRNGLIMRAVGDTMIISPPLVIDPSQIDELITLARKCLDQTAAAVLA

SEQ ID NO: 55 - protein, 378 aa, Bacillus subtilis
MIIGVPKEIKNNENRVALTPGGVSQLISNGHRVLVETGAGLGSGFENEAYESAGAEIIADPKQVWDAEMVMKVKEPLPEEYVYFRKGLVLFTYLHLAAEPELAQALKDKGVTAIAYETVSEGRTLPLLTPMSEVAGRMAAQIGAQFLEKPKGGKGILLAGVPGVSRGKVTIIGGGVVGTNAAKMAVGLGADVTIIDLNADRLRQLDDIFGHQIKTLISNPVNIADAVAEADLLICAVLIPGAKAPTLVTEEMVKQMKPGSVIVDVAIDQGGIVETVDHITTHDQPTYEKHGVVHYAVANMPGAVPRTSTIALTNVTVPYALQIANKGAVKALADNTALRAGLNTANGHVTYEAVARDLGYEYVPAEKALQDESSVAGA

SEQ ID NO: 56 - protein, 6 aa, synthetic construct
HHHHHH